(19) United States
(12) Patent Application Publication
(10) Pub. No.: US 2023/0073125 A1
(43) Pub. Date: Mar. 9, 2023

(54) PERIODONTAL MICROPATCH AND USES THEREOF

(71) Applicant: The Regents of the University of California, Oakland, CA (US)

(72) Inventors: Mohammad Mahdi HASANI-SADRABADI, Los Angeles, CA (US); Song LI, Beverly Hills, CA (US); Tara L. AGHALOO, Sherman Oaks, CA (US); Alireza MOSHAVERINIA, Los Angeles, CA (US)

(73) Assignee: The Regents of the University of California, Oakland, CA (US)

(21) Appl. No.: 17/772,418

(22) PCT Filed: Oct. 29, 2020
(86) PCT No.: PCT/US2020/058069

§ 371 Date: Sep. 22, 2022

Related U.S. Application Data
(60) Provisional application No. 62/927,564, filed on Oct. 29, 2019.

(51) Int. Cl. A61K 9/00; A61K 31/65; A61K 47/34; A61K 47/36; A61K 38/20; A61K 38/18; A61K 31/573; A61K 31/405; A61K 31/192; A61K 31/5415; A61M 37/00; A61K 47/42; A61P 31/04; A61P 1/02; A61P 29/00

(52) U.S. Cl. CPC A61K 9/0021; A61K 31/65; A61K 47/34; A61K 47/36; A61K 38/2026; A61K 38/1841; A61K 31/573; A61K 31/405; A61K 31/192; A61K 31/5415; A61M 37/0015; A61K 47/42; A61P 31/04; A61P 1/02; A61P 29/00; A61M 2037/0023; A61M 2210/0631; A61M 2037/0061

(57) ABSTRACT

A drug delivery system is described comprising a detachable support and a plurality of drug-loaded transdermal microneedles, for delivering drugs into gingival tissue for the treatment of periodontal disease.

### CROSS-REFERENCE TO RELATED APPLICATIONS

This application claims the benefit of and priority to U.S. Provisional Patent Application No. 62/895,821, filed Sep. 4, 2019, the contents of which are incorporated herein by reference in its entirety.

### FIELD OF THE INVENTION

The present invention relates to treatment of periodontal diseases using a micro-patch that comprises microneedles that deliver drugs to gingival tissues.

### BACKGROUND OF THE INVENTION

Periodontitis is a chronic destructive inflammatory disease, which is one of the most prevalent chronic infections in humans. Periodontal disease leads to destruction of the periodontium including alveolar bone, the periodontal ligament (PDL), and root cementum. If left untreated, periodontitis will result in progressive periodontal detachment and bone loss that may eventually lead to early tooth loss. More than 47% of adults aged 30 years and older are diagnosed with some form of periodontal disease. Furthermore, periodontal disease increases with age. More than 70% of adults of 65 years of age or older are diagnosed with some form of periodontal disease. Data from the National Institute of Dental and Craniofacial Research (National Institutes of Health) has revealed that nearly 90% of elderly populations (>70 years old) present at least a moderate level of periodontal-related diseases.

Once damaged, the periodontium has a limited capacity for regeneration. Effective treatment for periodontal disease is of outmost importance as the regeneration of periodontal tissues is difficult after their damage and tooth loss eventually occurs. The ultimate goal of periodontal therapy is the regeneration of all components of the periodontium. Over the past decade, strategies for periodontal repair have been mainly based on conventional administration of antibiotics, guided tissue regeneration (GTR), or application of cytokines, growth factors, or bioactive molecules. These methods have been utilized to reduce the bacterial infection and to decrease pocket depth, which may result in periodontium alveolar bone regeneration. The various types of biodegradable and non-biodegradable delivery platforms have been explored for periodontitis and gingivitis treatments. However, the systematic reviews of the outcomes of these procedures show inconsistencies in results and variable outcomes and the efficacy of the therapeutics remains to be addressed.

The human oral microbiome consists of a complex polymicrobial community, which dwells in specific niches within the oral cavity and forms biofilms (plaques) on teeth, prostheses and mucosal surfaces. The bacterial population colonizing healthy teeth or implants is similar and is mainly comprised of Gram-positive facultative cocci. The microbiota found in periodontitis is predominantly comprised of Gram-negative obligate anaerobes. The bacterial colonization of the periodontal pocket and the inflammatory response of the host cause periodontitis. Species most commonly associated with this disease include Porphyromonas gingivalis (P.g.) and Aggregatibacter actinomycetemcomitans (A.a.). Currently, most therapies deliver antibiotics to treat bacterium infection in gum tissues, but the drugs may not penetrate gingival tissues effectively. In addition, although microbial infection is the initial factor of the periodontitis, accumulation of destructive immune cells such as macrophages and T cells into the periodontium plays a critical role in the disease progression. While polarization of monocytes toward pro-inflammatory macrophages can promote defense against bacteria but secretion of anti-inflammatory cytokines and proteases will accelerate gingival tissue degeneration, alveolar bone resorption, and damage periodontal connective tissue.

Effective methods for treating highly prevalent gum and periodontal diseases are needed.

### SUMMARY OF THE INVENTION

In one aspect, a periodontal drug delivery system is provided comprising a microneedle patch, the microneedle patch comprising:

(a) a detachable support layer comprising a surface capable of a sealable application to a diseased periodontal tissue of a subject in need thereof; and
(b) a plurality of detachable, drug-loaded transdermal microneedles having bases disposed on the surface of the detachable support layer and projecting perpendicularly therefrom.

In one embodiment, the detachable support layer, the transdermal microneedles, or both, independently comprise a biodegradable polymer. In one embodiment, the biodegradable polymer is independently selected from the group consisting of chitosan, alginate, gelatin, hyaluronic acid (HA), polycaprolactone (PCL), poly(lactic-co-glycolic acid) (PLGA), hydrophobically-modified alginate, hydrophobically-modified chitosan, alginate-heparin, chitosan-heparin, methacrylated gelatin, and combinations thereof. In one embodiment, the biodegradable polymer dissolves at body temperature, the body temperature ranging from 97° F. (36.1° C.) to 99° F. (37.2° C.).

In one embodiment, the detachable support layer is non-membranous and non-adhesive. In one embodiment, the detachable support layer, the microneedles, or both are porous.

In one embodiment, the detachable support layer comprises a drug. In one embodiment, the drug is present within the detachable support layer, the drug is coated on the detachable support layer, of the combination thereof. In one embodiment, the transdermal microneedles are coated with a drug. In one embodiment, the drug-loaded transdermal microneedles have a height ranging from about 300 μm to about 1200 μm. In one embodiment, the drug-loaded transdermal microneedles comprises a base diameter of from about 100 μm to about 400 μm. In one embodiment, the drug-loaded transdermal microneedles have an aspect ratio of height to diameter of about 5 or more. In one embodiment, the plurality of drug-loaded transdermal microneedles comprises from 10 to 90 transdermal microneedles. In one embodiment, the plurality of drug-loaded transdermal microneedles comprises from 100 to 900 transdermal microneedles.

In one embodiment, the drug-loaded transdermal microneedles comprise an antibiotic, antibacterial agent, an anti-inflammatory agent, a growth factor, a cytokine, or any combination thereof. In one embodiment, the drug in the drug-loaded transdermal microneedles is soluble, is in nanoparticles, is in microparticles, or any combination thereof. In one embodiment, the drug in the detachable support layer comprises an antibiotic, antibacterial agent, an anti-inflammatory agent, a growth factor, a cytokine or any combination thereof. In one embodiment, the drug in the detachable support layer is soluble, is in nanoparticles is in microparticles, or any combination thereof.

In one embodiment, the drug coating the microneedles, the drug coating the detachable support, or both, are independently selected from an antibiotic, antibacterial agent, an anti-inflammatory agent, a growth factor, a cytokine or any combination thereof. In one embodiment, the drug is in nanoparticles or in microparticles.

In one embodiment, the drug in the drug-loaded transdermal microneedles, the drug in the detachable support layer, or both, are independently an antibiotic, antibacterial agent, an anti-inflammatory agent, a growth factor, a cytokine or any combination thereof. In one embodiment, the drug-loaded transdermal microneedles comprise an antibiotic, an anti-inflammatory agent, a cytokine, a growth factor, or a combination thereof, and the detachable support layer comprises an antibiotic. In one embodiment, the drug-loaded transdermal microneedles comprise microparticles comprising an anti-inflammatory agent, a cytokine, a growth factor, or a combination thereof, and the detachable support layer comprises an antibiotic.

In one embodiment, wherein the drug-loaded transdermal microneedles comprise an anti-inflammatory agent, and the detachable support comprises an antibiotic. In one embodiment, the drug-loaded transdermal microneedles comprise a cytokine, and the detachable support comprises an antibiotic. In one embodiment, the antibacterial agent is tetracycline, doxycycline, chlorhexidine and/or minocycline.

In one embodiment, an amount of from about 50 ng up to about 1,500 ng of the antibacterial agent is provided per transdermal microneedle patch.

In one embodiment, the anti-inflammatory agent is a steroid and/or a non-steroidal anti-inflammatory drug. In one embodiment, the steroid is dexamethasone hydrocortisone, cortisone, and/or prednisone. In one embodiment, the non-steroidal anti-inflammatory drug is indomethacin, naproxen, ibuprofen, flurbiprofen and/or piroxicam.

In one embodiment, the diseased periodontal tissue comprises periodontal ligament, cementum, gingiva, and/or alveolar bone. In one embodiment, the subject has periodontitis and/or gingivitis.

In one aspect, a method is provided for regenerating periodontal tissue in a subject in need thereof, the method comprising sealably applying to the periodontal tissue a microneedle patch, the microneedle patch comprising

(c) a detachable support layer comprising a surface capable of a sealable application to a diseased periodontal tissue of a subject in need thereof; and
(d) a plurality of detachable, drug-loaded transdermal microneedles having bases disposed on the surface of the detachable support layer and projecting perpendicularly therefrom.

In one embodiment, the detachable support layer, the transdermal microneedles, or both, independently comprise a biodegradable polymer. In one embodiment, the biodegradable polymer is independently selected from the group consisting of chitosan, alginate, gelatin, hyaluronic acid (HA), polycaprolactone (PCL), poly(lactic-co-glycolic acid) (PLGA), hydrophobically-modified alginate, hydrophobically-modified chitosan, alginate-heparin, chitosan-heparin, methacrylated gelatin, and combinations thereof. In one embodiment, the biodegradable polymer dissolves at body temperature, the body temperature ranging from 97° F. (36.1° C.) to 99° F. (37.2° C.).

In one embodiment, the detachable support layer is non-membranous and non-adhesive. In one embodiment, the detachable support layer, the microneedles, or both are porous.

In one embodiment, the detachable support layer comprises a drug. In one embodiment, the drug is present within the detachable support layer, the drug is coated on the detachable support layer, of the combination thereof. In one embodiment, the transdermal microneedles are coated with a drug. In one embodiment, the drug-loaded transdermal microneedles have a height ranging from about 300 μm to about 1200 μm. In one embodiment, the drug-loaded transdermal microneedles comprises a base diameter of from about 100 μm to about 400 μm. In one embodiment, the drug-loaded transdermal microneedles have an aspect ratio of height to diameter of about 5 or more. In one embodiment, the plurality of drug-loaded transdermal microneedles comprises from 10 to 90 transdermal microneedles. In one embodiment, the plurality of drug-loaded transdermal microneedles comprises from 100 to 900 transdermal microneedles.

In one embodiment, the drug-loaded transdermal microneedles comprise an antibiotic, antibacterial agent, an anti-inflammatory agent, a growth factor, a cytokine, or any combination thereof. In one embodiment, the drug in the drug-loaded transdermal microneedles is soluble, is in nanoparticles, is in microparticles, or any combination thereof. In one embodiment, the drug in the detachable support layer comprises an antibiotic, antibacterial agent, an anti-inflammatory agent, a growth factor, a cytokine or any combination thereof. In one embodiment, the drug in the detachable support layer is soluble, is in nanoparticles is in microparticles, or any combination thereof.

In one embodiment, the drug coating the microneedles, the drug coating the detachable support, or both, are independently selected from an antibiotic, antibacterial agent, an anti-inflammatory agent, a growth factor, a cytokine or any combination thereof. In one embodiment, the drug is in nanoparticles or in microparticles.

In one embodiment, the drug in the drug-loaded transdermal microneedles, the drug in the detachable support layer, or both, are independently an antibiotic, antibacterial agent, an anti-inflammatory agent, a growth factor, a cytokine or any combination thereof. In one embodiment, the drug-loaded transdermal microneedles comprise an antibiotic, an anti-inflammatory agent, a cytokine, a growth factor, or a combination thereof, and the detachable support layer comprises an antibiotic. In one embodiment, the drug-loaded transdermal microneedles comprise microparticles comprising an anti-inflammatory agent, a cytokine, a growth factor, or a combination thereof, and the detachable support layer comprises an antibiotic.

In one embodiment, wherein the drug-loaded transdermal microneedles comprise an anti-inflammatory agent, and the detachable support comprises an antibiotic. In one embodiment, the drug-loaded transdermal microneedles comprise a cytokine, and the detachable support comprises an antibiotic. In one embodiment, the antibacterial agent is tetracycline, doxycycline, chlorhexidine and/or minocycline.

In one embodiment, an amount of from about 50 ng up to about 1,500 ng of the antibacterial agent is provided per transdermal microneedle patch.

In one embodiment, the anti-inflammatory agent is a steroid and/or a non-steroidal anti-inflammatory drug. In one embodiment, the steroid is dexamethasone hydrocortisone, cortisone, and/or prednisone. In one embodiment, the non-steroidal anti-inflammatory drug is indomethacin, naproxen, ibuprofen, flurbiprofen and/or piroxicam.

In one embodiment, the diseased periodontal tissue comprises periodontal ligament, cementum, gingiva, and/or alveolar bone. In one embodiment, the subject has periodontitis and/or gingivitis.

In one aspect, a method is provided for reducing local inflammation in periodontal tissue of a subject in need thereof, the method comprising sealably applying to the periodontal tissue a microneedle patch, the microneedle patch comprising
(a) a detachable support layer comprising a surface capable of a sealable application to a diseased periodontal tissue of a subject in need thereof; and
(b) a plurality of detachable, drug-loaded transdermal microneedles having bases disposed on the surface of the detachable support layer and projecting perpendicularly therefrom.

In one embodiment, the detachable support layer, the transdermal microneedles, or both, independently comprise a biodegradable polymer. In one embodiment, the biodegradable polymer is independently selected from the group consisting of chitosan, alginate, gelatin, hyaluronic acid (HA), polycaprolactone (PCL), poly(lactic-co-glycolic acid) (PLGA), hydrophobically-modified alginate, hydrophobically-modified chitosan, alginate-heparin, chitosan-heparin, methacrylated gelatin, and combinations thereof. In one embodiment, the biodegradable polymer dissolves at body temperature, the body temperature ranging from 97° F. (36.1° C.) to 99° F. (37.2° C.).

In one embodiment, the detachable support layer is non-membranous and non-adhesive. In one embodiment, the detachable support layer, the microneedles, or both are porous.

In one embodiment, the detachable support layer comprises a drug. In one embodiment, the drug is present within the detachable support layer, the drug is coated on the detachable support layer, of the combination thereof. In one embodiment, the transdermal microneedles are coated with a drug. In one embodiment, the drug-loaded transdermal microneedles have a height ranging from about 300 μm to about 1200 μm. In one embodiment, the drug-loaded transdermal microneedles comprises a base diameter of from about 100 μm to about 400 μm. In one embodiment, the drug-loaded transdermal microneedles have an aspect ratio of height to diameter of about 5 or more. In one embodiment, the plurality of drug-loaded transdermal microneedles comprises from 10 to 90 transdermal microneedles. In one embodiment, the plurality of drug-loaded transdermal microneedles comprises from 100 to 900 transdermal microneedles.

In one embodiment, the drug-loaded transdermal microneedles comprise an antibiotic, antibacterial agent, an anti-inflammatory agent, a growth factor, a cytokine, or any combination thereof. In one embodiment, the drug in the drug-loaded transdermal microneedles is soluble, is in nanoparticles, is in microparticles, or any combination thereof. In one embodiment, the drug in the detachable support layer comprises an antibiotic, antibacterial agent, an anti-inflammatory agent, a growth factor, a cytokine or any combination thereof. In one embodiment, the drug in the detachable support layer is soluble, is in nanoparticles is in microparticles, or any combination thereof.

In one embodiment, the drug coating the microneedles, the drug coating the detachable support, or both, are independently selected from an antibiotic, antibacterial agent, an anti-inflammatory agent, a growth factor, a cytokine or any combination thereof. In one embodiment, the drug is in nanoparticles or in microparticles.

In one embodiment, the drug in the drug-loaded transdermal microneedles, the drug in the detachable support layer, or both, are independently an antibiotic, antibacterial agent, an anti-inflammatory agent, a growth factor, a cytokine or any combination thereof. In one embodiment, the drug-loaded transdermal microneedles comprise an antibiotic, an anti-inflammatory agent, a cytokine, a growth factor, or a combination thereof, and the detachable support layer comprises an antibiotic. In one embodiment, the drug-loaded transdermal microneedles comprise microparticles comprising an anti-inflammatory agent, a cytokine, a growth factor, or a combination thereof, and the detachable support layer comprises an antibiotic.

In one embodiment, wherein the drug-loaded transdermal microneedles comprise an anti-inflammatory agent, and the detachable support comprises an antibiotic. In one embodiment, the drug-loaded transdermal microneedles comprise a cytokine, and the detachable support comprises an antibiotic. In one embodiment, the antibacterial agent is tetracycline, doxycycline, chlorhexidine and/or minocycline.

In one embodiment, an amount of from about 50 ng up to about 1,500 ng of the antibacterial agent is provided per transdermal microneedle patch.

In one embodiment, the anti-inflammatory agent is a steroid and/or a non-steroidal anti-inflammatory drug. In one embodiment, the steroid is dexamethasone hydrocortisone, cortisone, and/or prednisone. In one embodiment, the non-steroidal anti-inflammatory drug is indomethacin, naproxen, ibuprofen, flurbiprofen and/or piroxicam.

In one embodiment, the diseased periodontal tissue comprises periodontal ligament, cementum, gingiva, and/or alveolar bone. In one embodiment, the subject has periodontitis and/or gingivitis.

In one aspect, a method is provided for promoting regeneration of bone loss in periodontal tissue of a subject in need thereof, the method comprising sealably applying to the periodontal tissue a microneedle patch, the microneedle patch comprising:

(a) a detachable support layer comprising a surface capable of a sealable application to a diseased periodontal tissue of a subject in need thereof; and
(b) a plurality of detachable, drug-loaded transdermal microneedles having bases disposed on the surface of the detachable support layer and projecting perpendicularly therefrom.

In one embodiment, the detachable support layer, the transdermal microneedles, or both, independently comprise a biodegradable polymer. In one embodiment, the biodegradable polymer is independently selected from the group consisting of chitosan, alginate, gelatin, hyaluronic acid (HA), polycaprolactone (PCL), poly(lactic-co-glycolic acid) (PLGA), hydrophobically-modified alginate, hydrophobically-modified chitosan, alginate-heparin, chitosan-heparin, methacrylated gelatin, and combinations thereof. In one embodiment, the biodegradable polymer dissolves at body temperature, the body temperature ranging from 97° F. (36.1° C.) to 99° F. (37.2° C.).

In one embodiment, the detachable support layer is non-membranous and non-adhesive. In one embodiment, the detachable support layer, the microneedles, or both are porous.

In one embodiment, the detachable support layer comprises a drug. In one embodiment, the drug is present within the detachable support layer, the drug is coated on the detachable support layer, of the combination thereof. In one embodiment, the transdermal microneedles are coated with a drug. In one embodiment, the drug-loaded transdermal microneedles have a height ranging from about 300 μm to about 1200 μm. In one embodiment, the drug-loaded transdermal microneedles comprises a base diameter of from about 100 μm to about 400 μm. In one embodiment, the drug-loaded transdermal microneedles have an aspect ratio of height to diameter of about 5 or more. In one embodiment, the plurality of drug-loaded transdermal microneedles comprises from 10 to 90 transdermal microneedles. In one embodiment, the plurality of drug-loaded transdermal microneedles comprises from 100 to 900 transdermal microneedles.

In one embodiment, the drug-loaded transdermal microneedles comprise an antibiotic, antibacterial agent, an anti-inflammatory agent, a growth factor, a cytokine, or any combination thereof. In one embodiment, the drug in the drug-loaded transdermal microneedles is soluble, is in nanoparticles, is in microparticles, or any combination thereof. In one embodiment, the drug in the detachable support layer comprises an antibiotic, antibacterial agent, an anti-inflammatory agent, a growth factor, a cytokine or any combination thereof. In one embodiment, the drug in the detachable support layer is soluble, is in nanoparticles is in microparticles, or any combination thereof.

In one embodiment, the drug coating the microneedles, the drug coating the detachable support, or both, are independently selected from an antibiotic, antibacterial agent, an anti-inflammatory agent, a growth factor, a cytokine or any combination thereof. In one embodiment, the drug is in nanoparticles or in microparticles.

In one embodiment, the drug in the drug-loaded transdermal microneedles, the drug in the detachable support layer, or both, are independently an antibiotic, antibacterial agent, an anti-inflammatory agent, a growth factor, a cytokine or any combination thereof. In one embodiment, the drug-loaded transdermal microneedles comprise an antibiotic, an anti-inflammatory agent, a cytokine, a growth factor, or a combination thereof, and the detachable support layer comprises an antibiotic. In one embodiment, the drug-loaded transdermal microneedles comprise microparticles comprising an anti-inflammatory agent, a cytokine, a growth factor, or a combination thereof, and the detachable support layer comprises an antibiotic.

In one embodiment, wherein the drug-loaded transdermal microneedles comprise an anti-inflammatory agent, and the detachable support comprises an antibiotic. In one embodiment, the drug-loaded transdermal microneedles comprise a cytokine, and the detachable support comprises an antibiotic. In one embodiment, the antibacterial agent is tetracycline, doxycycline, chlorhexidine and/or minocycline.

In one embodiment, an amount of from about 50 ng up to about 1,500 ng of the antibacterial agent is provided per transdermal microneedle patch.

In one embodiment, the anti-inflammatory agent is a steroid and/or a non-steroidal anti-inflammatory drug. In one embodiment, the steroid is dexamethasone hydrocortisone, cortisone, and/or prednisone. In one embodiment, the non-steroidal anti-inflammatory drug is indomethacin, naproxen, ibuprofen, flurbiprofen and/or piroxicam.

In one embodiment, the diseased periodontal tissue comprises periodontal ligament, cementum, gingiva, and/or alveolar bone. In one embodiment, the subject has periodontitis and/or gingivitis.

In one aspect, a method is provided for treating periodontitis and/or gingivitis in a subject in need thereof, the method comprising sealably applying to the periodontal tissue a microneedle patch, the microneedle patch comprising:

(a) a detachable support layer comprising a surface capable of a sealable application to a diseased periodontal tissue of a subject in need thereof; and
(b) a plurality of detachable, drug-loaded transdermal microneedles having bases disposed on the surface of the detachable support layer and projecting perpendicularly therefrom.

In one embodiment, the detachable support layer, the transdermal microneedles, or both, independently comprise a biodegradable polymer. In one embodiment, the biodegradable polymer is independently selected from the group consisting of chitosan, alginate, gelatin, hyaluronic acid (HA), polycaprolactone (PCL), poly(lactic-co-glycolic acid) (PLGA), hydrophobically-modified alginate, hydrophobically-modified chitosan, alginate-heparin, chitosan-heparin, methacrylated gelatin, and combinations thereof. In one embodiment, the biodegradable polymer dissolves at body temperature, the body temperature ranging from 97° F. (36.1° C.) to 99° F. (37.2° C.).

In one embodiment, the detachable support layer is non-membranous and non-adhesive. In one embodiment, the detachable support layer, the microneedles, or both are porous.

In one embodiment, the detachable support layer comprises a drug. In one embodiment, the drug is present within the detachable support layer, the drug is coated on the detachable support layer, of the combination thereof. In one embodiment, the transdermal microneedles are coated with a drug. In one embodiment, the drug-loaded transdermal microneedles have a height ranging from about 300 μm to about 1200 μm. In one embodiment, the drug-loaded transdermal microneedles comprise a base diameter of from about 100 μm to about 400 μm. In one embodiment, the drug-loaded transdermal microneedles have an aspect ratio of height to diameter of about 5 or more. In one embodiment, the plurality of drug-loaded transdermal microneedles comprises from 10 to 90 transdermal microneedles. In one embodiment, the plurality of drug-loaded transdermal microneedles comprises from 100 to 900 transdermal microneedles.

In one embodiment, the drug-loaded transdermal microneedles comprise an antibiotic, antibacterial agent, an anti-inflammatory agent, a growth factor, a cytokine, or any combination thereof. In one embodiment, the drug in the drug-loaded transdermal microneedles is soluble, is in nanoparticles, is in microparticles, or any combination thereof. In one embodiment, the drug in the detachable support layer comprises an antibiotic, antibacterial agent, an anti-inflammatory agent, a growth factor, a cytokine or any combination thereof. In one embodiment, the drug in the detachable support layer is soluble, is in nanoparticles is in microparticles, or any combination thereof.

In one embodiment, the drug coating the microneedles, the drug coating the detachable support, or both, are independently selected from an antibiotic, antibacterial agent, an anti-inflammatory agent, a growth factor, a cytokine or any combination thereof. In one embodiment, the drug is in nanoparticles or in microparticles.

In one embodiment, the drug in the drug-loaded transdermal microneedles, the drug in the detachable support layer, or both, are independently an antibiotic, antibacterial agent, an anti-inflammatory agent, a growth factor, a cytokine or any combination thereof. In one embodiment, the drug-loaded transdermal microneedles comprise an antibiotic, an anti-inflammatory agent, a cytokine, a growth factor, or a combination thereof, and the detachable support layer comprises an antibiotic. In one embodiment, the drug-loaded transdermal microneedles comprise microparticles comprising an anti-inflammatory agent, a cytokine, a growth factor, or a combination thereof, and the detachable support layer comprises an antibiotic.

In one embodiment, wherein the drug-loaded transdermal microneedles comprise an anti-inflammatory agent, and the detachable support comprises an antibiotic. In one embodiment, the drug-loaded transdermal microneedles comprise a cytokine, and the detachable support comprises an antibiotic. In one embodiment, the antibacterial agent is tetracycline, doxycycline, chlorhexidine and/or minocycline.

In one embodiment, an amount of from about 50 ng up to about 1,500 ng of the antibacterial agent is provided per transdermal microneedle patch.

In one embodiment, the anti-inflammatory agent is a steroid and/or a non-steroidal anti-inflammatory drug. In one embodiment, the steroid is dexamethasone hydrocortisone, cortisone, and/or prednisone. In one embodiment, the non-steroidal anti-inflammatory drug is indomethacin, naproxen, ibuprofen, flurbiprofen and/or piroxicam.

In one embodiment, the diseased periodontal tissue comprises periodontal ligament, cementum, gingiva, and/or alveolar bone.

In one aspect, provided is a periodontal drug delivery system comprising a microneedle patch, the microneedle patch comprising: (a) a detachable support layer comprising a surface capable of a sealable application to a diseased periodontal tissue of a subject in need thereof, wherein the detachable support layer comprises a first drug, and the detachable support layer is formulated for immediate release of the first drug; and (b) a plurality of detachable, transdermal microneedles having bases disposed on the surface of the detachable support layer and projecting perpendicularly therefrom, wherein the transdermal microneedles comprise nano/microparticles loaded with a second drug, and the transdermal microneedles are formulated for sustained release of the second drug.

In an embodiment, the detachable support layer, the transdermal microneedles, or both, independently comprise a biodegradable polymer. In some embodiments of the periodontal drug delivery system the biodegradable polymer is independently selected from the group consisting of chitosan, alginate, gelatin, hyaluronic acid (HA), polycaprolactone (PCL), poly(lactic-co-glycolic acid) (PLGA), hydrophobically-modified alginate, hydrophobically-modified chitosan, alginate-heparin, chitosan-heparin, methacrylated gelatin (GelMA), and combinations thereof. In an embodiment, the biodegradable polymer dissolves at body temperature, the body temperature ranging from 97° F. (36.1° C.) to 99° F. (37.2° C.). In an embodiment, the detachable support layer is non-membranous and non-adhesive. In certain embodiments, the detachable support layer, the microneedles, or both are porous. In a particular embodiment, the first drug is an antibiotic or an antibacterial agent. In an embodiment, the first drug is present within the detachable support layer, the first drug is coated on the detachable support layer, or a combination thereof. In some embodiments, the first drug is dispersed within a biodegradable polymer. In an embodiment, the nano/microparticles loaded with the second drug are dispersed within a biodegradable polymer. In a particular embodiment, the second drug is the same drug as the first drug, is a different drug than the first drug, or combinations thereof. In some embodiments, the different drug is a small molecule, an antibiotic, an immunomodulatory cytokine, a growth factor or combinations thereof. In an embodiment, the immunomodulatory cytokine is IL-4 and/or IL-10 and the growth factor is transforming growth factor beta (TGF-β). In an embodiment, the small molecule, antibiotic, immunomodulatory cytokine growth factor or combinations thereof is encapsulated in the nano/microparticles, wherein the encapsulated nano/microparticles are dispersed within a biopolymer. In one embodiment, the biopolymer comprises alginate and calcium chloride. In an embodiment, the alginate is a monodisperse heparin-functionalized alginate. In an embodiment, the biopolymer comprises heparin-functionalized mesoporous silica microparticles. In one embodiment, the monodisperse heparin-functionalized alginate is an encapsulated alginate-heparin microparticle. In some embodiments, the encapsulated alginate-heparin microparticle is dispersed in a second biopolymer. In an embodiment, the second biopolymer is alginate, GelMA or a combination thereof. In a particular embodiment, the immunomodulatory cytokine and the growth factor reprogram M1 and M0 macrophages toward M2 macrophages. In some embodiments, the sustained release of the second drug is a release of from 2 weeks to 8 weeks. In one embodiment, the sustained release of the second drug is a release of from 2 weeks to 4 weeks. In some embodiments, the drug-loaded transdermal microneedles have a height ranging from about 300 μm to about 1200 μm. In one embodiment, the drug-loaded transdermal microneedles comprise a base diameter of from about 100 μm to about 400 μm. In an embodiment, the drug-loaded transdermal microneedles have an aspect ratio of height to diameter of about 5 or more. In an embodiment, the plurality of drug-loaded transdermal microneedles comprises from 10 to 90 transdermal microneedles. In an embodiment, the plurality of drug-loaded transdermal microneedles comprises from 100 to 900 transdermal microneedles. In a particular embodiment, the transdermal microneedles loaded with the second drug comprise an antibiotic, antibacterial agent, an anti-inflammatory agent, a growth factor, a cytokine, or any combination thereof. In one embodiment, the second drug in the drug-loaded transdermal microneedles is soluble, is in nanoparticles, is in microparticles, or any combination thereof. In an embodiment, the first drug in the detachable support layer comprises an antibiotic, antibacterial agent, an anti-inflammatory agent, a growth factor, a cytokine or any combination thereof. In some embodiments, the first drug in the detachable support layer is soluble, is in nanoparticles is in microparticles, or any combination thereof. In a particular embodiment, the first drug and/or the second drug is coated on the microneedles, the first drug and/or the second drug is coated on the detachable support, or both, and the first drug and/or the second are independently selected from an antibiotic, antibacterial agent, an anti-inflammatory agent, a growth factor, a cytokine or any combination thereof. In an embodiment, the first drug and/or the second drug is in nanoparticles or in microparticles. In one embodiment, the second drug in the drug-loaded transdermal microneedles, is independently an antibiotic, antibacterial agent, an anti-inflammatory agent, a growth factor, a cytokine or any combination thereof. In one embodiment, the second drug loaded in the transdermal microneedles comprises an antibiotic, an anti-inflammatory agent, a cytokine, a growth factor, or a combination thereof, and the detachable support layer comprises an antibiotic. In an embodiment, the second drug loaded in the transdermal microneedles comprises microparticles comprising an anti-inflammatory agent, a cytokine, a growth factor, or a combination thereof, and the detachable support layer comprises an antibiotic. In some embodiments, the second drug loaded in the transdermal microneedles comprises an anti-inflammatory agent, and the detachable support comprises an antibiotic. In an embodiment, the second drug loaded in the transdermal microneedles comprise a cytokine, and the detachable support comprises an antibiotic. In a particular embodiment, the antibiotic or antibacterial agent is tetracycline, doxycycline, chlorhexidine and/or minocycline. In an embodiment, the periodontal drug delivery system comprises an amount of from about 50 ng up to about 1,500 ng of the antibacterial agent per transdermal microneedle patch. In an embodiment of the periodontal drug delivery system, the anti-inflammatory agent is a steroid and/or a non-steroidal anti-inflammatory drug. In one embodiment, the steroid is dexamethasone hydrocortisone, cortisone, and/or prednisone. In an embodiment, the non-steroidal anti-inflammatory drug is indomethacin, naproxen, ibuprofen, flurbiprofen and/or piroxicam. In some embodiments, the periodontal drug delivery system is administered to the subject, wherein the diseased periodontal tissue comprises periodontal ligament, cementum, gingiva, and/or alveolar bone. In one embodiment, the subject has periodontitis and/or gingivitis.

In another aspect, provided is a method for regenerating periodontal tissue in a subject in need thereof, the method comprising sealably applying to the periodontal tissue a microneedle patch, the microneedle patch comprising: (a) a detachable support layer comprising a surface capable of a sealable application to a diseased periodontal tissue of a subject in need thereof, wherein the detachable support layer comprises a first drug, and the detachable support layer is formulated for immediate release of the first drug; and (b) a plurality of detachable, transdermal microneedles having bases disposed on the surface of the detachable support layer and projecting perpendicularly therefrom, wherein the transdermal microneedles comprise nano/microparticles loaded with a second drug, and the transdermal microneedles are formulated for sustained release of the second drug.

In an embodiment, the detachable support layer, the transdermal microneedles, or both, independently comprise a biodegradable polymer. In some embodiments of the periodontal drug delivery system the biodegradable polymer is independently selected from the group consisting of chitosan, alginate, gelatin, hyaluronic acid (HA), polycaprolactone (PCL), poly(lactic-co-glycolic acid) (PLGA), hydrophobically-modified alginate, hydrophobically-modified chitosan, alginate-heparin, chitosan-heparin, methacrylated gelatin (GelMA), and combinations thereof. In an embodiment, the biodegradable polymer dissolves at body temperature, the body temperature ranging from 97° F. (36.1° C.) to 99° F. (37.2° C.). In an embodiment, the detachable support layer is non-membranous and non-adhesive. In certain embodiments, the detachable support layer, the microneedles, or both are porous. In a particular embodiment, the first drug is an antibiotic or an antibacterial agent. In an embodiment, the first drug is present within the detachable support layer, the first drug is coated on the detachable support layer, or a combination thereof. In some embodiments, the first drug is dispersed within a biodegradable polymer. In an embodiment, the nano/microparticles loaded with the second drug are dispersed within a biodegradable polymer. In a particular embodiment, the second drug is the same drug as the first drug, is a different drug than the first drug, or combinations thereof. In some embodiments, the different drug is a small molecule, an antibiotic, an immunomodulatory cytokine, a growth factor or combinations thereof. In an embodiment, the immunomodulatory cytokine is IL-4 and/or IL-10 and the growth factor is transforming growth factor beta (TGF-β). In an embodiment, the small molecule, antibiotic, immunomodulatory cytokine growth factor or combinations thereof is encapsulated in the nano/microparticles, wherein the encapsulated nano/microparticles are dispersed within a biopolymer. In one embodiment, the biopolymer comprises alginate and calcium chloride. In an embodiment, the alginate is a monodisperse heparin-functionalized alginate. In an embodiment, the biopolymer comprises heparin-functionalized mesoporous silica microparticles. In one embodiment, the monodisperse heparin-functionalized alginate is an encapsulated alginate-heparin microparticle. In some embodiments, the encapsulated alginate-heparin microparticle is dispersed in a second biopolymer. In an embodiment, the second biopolymer is alginate, GelMA or a combination thereof. In a particular embodiment, the immunomodulatory cytokine and the growth factor reprogram M1 and M0 macrophages toward M2 macrophages. In some embodiments, the sustained release of the second drug is a release of from 2 weeks to 8 weeks. In one embodiment, the sustained release of the second drug is a release of from 2 weeks to 4 weeks. In some embodiments, the drug-loaded transdermal microneedles have a height ranging from about 300 μm to about 1200 μm. In one embodiment, the drug-loaded transdermal microneedles comprise a base diameter of from about 100 μm to about 400 μm. In an embodiment, the drug-loaded transdermal microneedles have an aspect ratio of height to diameter of about 5 or more. In an embodiment, the plurality of drug-loaded transdermal microneedles comprises from 10 to 90 transdermal microneedles. In an embodiment, the plurality of drug-loaded transdermal microneedles comprises from 100 to 900 transdermal microneedles. In a particular embodiment, the transdermal microneedles loaded with the second drug comprise an antibiotic, antibacterial agent, an anti-inflammatory agent, a growth factor, a cytokine, or any combination thereof. In one embodiment, the second drug in the drug-loaded transdermal microneedles is soluble, is in nanoparticles, is in microparticles, or any combination thereof. In an embodiment, the first drug in the detachable support layer comprises an antibiotic, antibacterial agent, an anti-inflammatory agent, a growth factor, a cytokine or any combination thereof. In some embodiments, the first drug in the detachable support layer is soluble, is in nanoparticles is in microparticles, or any combination thereof. In a particular embodiment, the first drug and/or the second drug is coated on the microneedles, the first drug and/or the second drug is coated on the detachable support, or both, and the first drug and/or the second are independently selected from an antibiotic, antibacterial agent, an anti-inflammatory agent, a growth factor, a cytokine or any combination thereof. In an embodiment, the first drug and/or the second drug is in nanoparticles or in microparticles. In one embodiment, the second drug in the drug-loaded transdermal microneedles, is independently an antibiotic, antibacterial agent, an anti-inflammatory agent, a growth factor, a cytokine or any combination thereof. In one embodiment, the second drug loaded in the transdermal microneedles comprises an antibiotic, an anti-inflammatory agent, a cytokine, a growth factor, or a combination thereof, and the detachable support layer comprises an antibiotic. In an embodiment, the second drug loaded in the transdermal microneedles comprises microparticles comprising an anti-inflammatory agent, a cytokine, a growth factor, or a combination thereof, and the detachable support layer comprises an antibiotic. In some embodiments, the second drug loaded in the transdermal microneedles comprises an anti-inflammatory agent, and the detachable support comprises an antibiotic. In an embodiment, the second drug loaded in the transdermal microneedles comprise a cytokine, and the detachable support comprises an antibiotic. In a particular embodiment, the antibiotic or antibacterial agent is tetracycline, doxycycline, chlorhexidine and/or minocycline. In an embodiment, the periodontal drug delivery system comprises an amount of from about 50 ng up to about 1,500 ng of the antibacterial agent per transdermal microneedle patch. In an embodiment of the periodontal drug delivery system, the anti-inflammatory agent is a steroid and/or a non-steroidal anti-inflammatory drug. In one embodiment, the steroid is dexamethasone hydrocortisone, cortisone, and/or prednisone. In an embodiment, the non-steroidal anti-inflammatory drug is indomethacin, naproxen, ibuprofen, flurbiprofen and/or piroxicam. In some embodiments, the periodontal drug delivery system is administered to the subject, wherein the diseased periodontal tissue comprises periodontal ligament, cementum, gingiva, and/or alveolar bone. In one embodiment, the subject has periodontitis and/or gingivitis.

In one aspect, provided is a method for reducing local inflammation in periodontal tissue of a subject in need thereof, the method comprising sealably applying to the periodontal tissue a microneedle patch, the microneedle patch comprising: (a) a detachable support layer comprising a surface capable of a sealable application to a diseased periodontal tissue of a subject in need thereof, wherein the detachable support layer comprises a first drug, and the detachable support layer is formulated for immediate release of the first drug; and (b) a plurality of detachable, transdermal microneedles having bases disposed on the surface of the detachable support layer and projecting perpendicularly therefrom, wherein the transdermal microneedles comprise nano/microparticles loaded with a second drug, and the transdermal microneedles are formulated for sustained release of the second drug.

In an embodiment, the detachable support layer, the transdermal microneedles, or both, independently comprise a biodegradable polymer. In some embodiments of the periodontal drug delivery system the biodegradable polymer is independently selected from the group consisting of chitosan, alginate, gelatin, hyaluronic acid (HA), polycaprolactone (PCL), poly(lactic-co-glycolic acid) (PLGA), hydrophobically-modified alginate, hydrophobically-modified chitosan, alginate-heparin, chitosan-heparin, methacrylated gelatin (GelMA), and combinations thereof. In an embodiment, the biodegradable polymer dissolves at body temperature, the body temperature ranging from 97° F. (36.1° C.) to 99° F. (37.2° C.). In an embodiment, the detachable support layer is non-membranous and non-adhesive. In certain embodiments, the detachable support layer, the microneedles, or both are porous. In a particular embodiment, the first drug is an antibiotic or an antibacterial agent. In an embodiment, the first drug is present within the detachable support layer, the first drug is coated on the detachable support layer, or a combination thereof. In some embodiments, the first drug is dispersed within a biodegradable polymer. In an embodiment, the nano/microparticles loaded with the second drug are dispersed within a biodegradable polymer. In a particular embodiment, the second drug is the same drug as the first drug, is a different drug than the first drug, or combinations thereof. In some embodiments, the different drug is a small molecule, an antibiotic, an immunomodulatory cytokine, a growth factor or combinations thereof. In an embodiment, the immunomodulatory cytokine is IL-4 and/or IL-10 and the growth factor is transforming growth factor beta (TGF-β). In an embodiment, the small molecule, antibiotic, immunomodulatory cytokine growth factor or combinations thereof is encapsulated in the nano/microparticles, wherein the encapsulated nano/microparticles are dispersed within a biopolymer. In one embodiment, the biopolymer comprises alginate and calcium chloride. In an embodiment, the alginate is a monodisperse heparin-functionalized alginate. In an embodiment, the biopolymer comprises heparin-functionalized mesoporous silica microparticles. In one embodiment, the monodisperse heparin-functionalized alginate is an encapsulated alginate-heparin microparticle. In some embodiments, the encapsulated alginate-heparin microparticle is dispersed in a second biopolymer. In an embodiment, the second biopolymer is alginate, GelMA or a combination thereof. In a particular embodiment, the immunomodulatory cytokine and the growth factor reprogram M1 and M0 macrophages toward M2 macrophages. In some embodiments, the sustained release of the second drug is a release of from 2 weeks to 8 weeks. In one embodiment, the sustained release of the second drug is a release of from 2 weeks to 4 weeks. In some embodiments, the drug-loaded transdermal microneedles have a height ranging from about 300 μm to about 1200 μm. In one embodiment, the drug-loaded transdermal microneedles comprise a base diameter of from about 100 μm to about 400 μm. In an embodiment, the drug-loaded transdermal microneedles have an aspect ratio of height to diameter of about 5 or more. In an embodiment, the plurality of drug-loaded transdermal microneedles comprises from 10 to 90 transdermal microneedles. In an embodiment, the plurality of drug-loaded transdermal microneedles comprises from 100 to 900 transdermal microneedles. In a particular embodiment, the transdermal microneedles loaded with the second drug comprise an antibiotic, antibacterial agent, an anti-inflammatory agent, a growth factor, a cytokine, or any combination thereof. In one embodiment, the second drug in the drug-loaded transdermal microneedles is soluble, is in nanoparticles, is in microparticles, or any combination thereof. In an embodiment, the first drug in the detachable support layer comprises an antibiotic, antibacterial agent, an anti-inflammatory agent, a growth factor, a cytokine or any combination thereof. In some embodiments, the first drug in the detachable support layer is soluble, is in nanoparticles is in microparticles, or any combination thereof. In a particular embodiment, the first drug and/or the second drug is coated on the microneedles, the first drug and/or the second drug is coated on the detachable support, or both, and the first drug and/or the second are independently selected from an antibiotic, antibacterial agent, an anti-inflammatory agent, a growth factor, a cytokine or any combination thereof. In an embodiment, the first drug and/or the second drug is in nanoparticles or in microparticles. In one embodiment, the second drug in the drug-loaded transdermal microneedles, is independently an antibiotic, antibacterial agent, an anti-inflammatory agent, a growth factor, a cytokine or any combination thereof. In one embodiment, the second drug loaded in the transdermal microneedles comprises an antibiotic, an anti-inflammatory agent, a cytokine, a growth factor, or a combination thereof, and the detachable support layer comprises an antibiotic. In an embodiment, the second drug loaded in the transdermal microneedles comprises microparticles comprising an anti-inflammatory agent, a cytokine, a growth factor, or a combination thereof, and the detachable support layer comprises an antibiotic. In some embodiments, the second drug loaded in the transdermal microneedles comprises an anti-inflammatory agent, and the detachable support comprises an antibiotic. In an embodiment, the second drug loaded in the transdermal microneedles comprise a cytokine, and the detachable support comprises an antibiotic. In a particular embodiment, the antibiotic or antibacterial agent is tetracycline, doxycycline, chlorhexidine and/or minocycline. In an embodiment, the periodontal drug delivery system comprises an amount of from about 50 ng up to about 1,500 ng of the antibacterial agent per transdermal microneedle patch. In an embodiment of the periodontal drug delivery system, the anti-inflammatory agent is a steroid and/or a non-steroidal anti-inflammatory drug. In one embodiment, the steroid is dexamethasone hydrocortisone, cortisone, and/or prednisone. In an embodiment, the non-steroidal anti-inflammatory drug is indomethacin, naproxen, ibuprofen, flurbiprofen and/or piroxicam. In some embodiments, the periodontal drug delivery system is administered to the subject, wherein the diseased periodontal tissue comprises periodontal ligament, cementum, gingiva, and/or alveolar bone. In one embodiment, the subject has periodontitis and/or gingivitis.

In another aspect, provided is a method for promoting regeneration of bone loss in periodontal tissue of a subject in need thereof, the method comprising sealably applying to the periodontal tissue a microneedle patch, the microneedle patch comprising: (a) a detachable support layer comprising a surface capable of a sealable application to a diseased periodontal tissue of a subject in need thereof; and (b) a plurality of detachable, drug-loaded transdermal microneedles having bases disposed on the surface of the detachable support layer and projecting perpendicularly therefrom.

In an embodiment, the detachable support layer, the transdermal microneedles, or both, independently comprise a biodegradable polymer. In some embodiments of the periodontal drug delivery system the biodegradable polymer is independently selected from the group consisting of chitosan, alginate, gelatin, hyaluronic acid (HA), polycaprolactone (PCL), poly(lactic-co-glycolic acid) (PLGA), hydrophobically-modified alginate, hydrophobically-modified chitosan, alginate-heparin, chitosan-heparin, methacrylated gelatin (GelMA), and combinations thereof. In an embodiment, the biodegradable polymer dissolves at body temperature, the body temperature ranging from 97° F. (36.1° C.) to 99° F. (37.2° C.). In an embodiment, the detachable support layer is non-membranous and non-adhesive. In certain embodiments, the detachable support layer, the microneedles, or both are porous. In a particular embodiment, the first drug is an antibiotic or an antibacterial agent. In an embodiment, the first drug is present within the detachable support layer, the first drug is coated on the detachable support layer, or a combination thereof. In some embodiments, the first drug is dispersed within a biodegradable polymer. In an embodiment, the nano/microparticles loaded with the second drug are dispersed within a biodegradable polymer. In a particular embodiment, the second drug is the same drug as the first drug, is a different drug than the first drug, or combinations thereof. In some embodiments, the different drug is a small molecule, an antibiotic, an immunomodulatory cytokine, a growth factor or combinations thereof. In an embodiment, the immunomodulatory cytokine is IL-4 and/or IL-10 and the growth factor is transforming growth factor beta (TGF-β). In an embodiment, the small molecule, antibiotic, immunomodulatory cytokine growth factor or combinations thereof is encapsulated in the nano/microparticles, wherein the encapsulated nano/microparticles are dispersed within a biopolymer. In one embodiment, the biopolymer comprises alginate and calcium chloride. In an embodiment, the alginate is a monodisperse heparin-functionalized alginate. In an embodiment, the biopolymer comprises heparin-functionalized mesoporous silica microparticles. In one embodiment, the monodisperse heparin-functionalized alginate is an encapsulated alginate-heparin microparticle. In some embodiments, the encapsulated alginate-heparin microparticle is dispersed in a second biopolymer. In an embodiment, the second biopolymer is alginate, GelMA or a combination thereof. In a particular embodiment, the immunomodulatory cytokine and the growth factor reprogram M1 and M0 macrophages toward M2 macrophages. In some embodiments, the sustained release of the second drug is a release of from 2 weeks to 8 weeks. In one embodiment, the sustained release of the second drug is a release of from 2 weeks to 4 weeks. In some embodiments, the drug-loaded transdermal microneedles have a height ranging from about 300 μm to about 1200 μm. In one embodiment, the drug-loaded transdermal microneedles comprise a base diameter of from about 100 μm to about 400 μm. In an embodiment, the drug-loaded transdermal microneedles have an aspect ratio of height to diameter of about 5 or more. In an embodiment, the plurality of drug-loaded transdermal microneedles comprises from 10 to 90 transdermal microneedles. In an embodiment, the plurality of drug-loaded transdermal microneedles comprises from 100 to 900 transdermal microneedles. In a particular embodiment, the transdermal microneedles loaded with the second drug comprise an antibiotic, antibacterial agent, an anti-inflammatory agent, a growth factor, a cytokine, or any combination thereof. In one embodiment, the second drug in the drug-loaded transdermal microneedles is soluble, is in nanoparticles, is in microparticles, or any combination thereof. In an embodiment, the first drug in the detachable support layer comprises an antibiotic, antibacterial agent, an anti-inflammatory agent, a growth factor, a cytokine or any combination thereof. In some embodiments, the first drug in the detachable support layer is soluble, is in nanoparticles is in microparticles, or any combination thereof. In a particular embodiment, the first drug and/or the second drug is coated on the microneedles, the first drug and/or the second drug is coated on the detachable support, or both, and the first drug and/or the second are independently selected from an antibiotic, antibacterial agent, an anti-inflammatory agent, a growth factor, a cytokine or any combination thereof. In an embodiment, the first drug and/or the second drug is in nanoparticles or in microparticles. In one embodiment, the second drug in the drug-loaded transdermal microneedles, is independently an antibiotic, antibacterial agent, an anti-inflammatory agent, a growth factor, a cytokine or any combination thereof. In one embodiment, the second drug loaded in the transdermal microneedles comprises an antibiotic, an anti-inflammatory agent, a cytokine, a growth factor, or a combination thereof, and the detachable support layer comprises an antibiotic. In an embodiment, the second drug loaded in the transdermal microneedles comprises microparticles comprising an anti-inflammatory agent, a cytokine, a growth factor, or a combination thereof, and the detachable support layer comprises an antibiotic. In some embodiments, the second drug loaded in the transdermal microneedles comprises an anti-inflammatory agent, and the detachable support comprises an antibiotic. In an embodiment, the second drug loaded in the transdermal microneedles comprise a cytokine, and the detachable support comprises an antibiotic. In a particular embodiment, the antibiotic or antibacterial agent is tetracycline, doxycycline, chlorhexidine and/or minocycline. In an embodiment, the periodontal drug delivery system comprises an amount of from about 50 ng up to about 1,500 ng of the antibacterial agent per transdermal microneedle patch. In an embodiment of the periodontal drug delivery system, the anti-inflammatory agent is a steroid and/or a non-steroidal anti-inflammatory drug. In one embodiment, the steroid is dexamethasone hydrocortisone, cortisone, and/or prednisone. In an embodiment, the non-steroidal anti-inflammatory drug is indomethacin, naproxen, ibuprofen, flurbiprofen and/or piroxicam. In some embodiments, the periodontal drug delivery system is administered to the subject, wherein the diseased periodontal tissue comprises periodontal ligament, cementum, gingiva, and/or alveolar bone. In one embodiment, the subject has periodontitis and/or gingivitis.

In one aspect, provided is a method for treating periodontitis and/or gingivitis in a subject in need thereof, the method comprising sealably applying to the periodontal tissue a microneedle patch, the microneedle patch comprising: (a) a detachable support layer comprising a surface capable of a sealable application to a diseased periodontal tissue of a subject in need thereof; and (b) a plurality of detachable, drug-loaded transdermal microneedles having bases disposed on the surface of the detachable support layer and projecting perpendicularly therefrom.

In an embodiment, the detachable support layer, the transdermal microneedles, or both, independently comprise a biodegradable polymer. In some embodiments of the periodontal drug delivery system the biodegradable polymer is independently selected from the group consisting of chitosan, alginate, gelatin, hyaluronic acid (HA), polycaprolactone (PCL), poly(lactic-co-glycolic acid) (PLGA), hydrophobically-modified alginate, hydrophobically-modified chitosan, alginate-heparin, chitosan-heparin, methacrylated gelatin (GelMA), and combinations thereof. In an embodiment, the biodegradable polymer dissolves at body temperature, the body temperature ranging from 97° F. (36.1° C.) to 99° F. (37.2° C.). In an embodiment, the detachable support layer is non-membranous and non-adhesive. In certain embodiments, the detachable support layer, the microneedles, or both are porous. In a particular embodiment, the first drug is an antibiotic or an antibacterial agent. In an embodiment, the first drug is present within the detachable support layer, the first drug is coated on the detachable support layer, or a combination thereof. In some embodiments, the first drug is dispersed within a biodegradable polymer. In an embodiment, the nano/microparticles loaded with the second drug are dispersed within a biodegradable polymer. In a particular embodiment, the second drug is the same drug as the first drug, is a different drug than the first drug, or combinations thereof. In some embodiments, the different drug is a small molecule, an antibiotic, an immunomodulatory cytokine, a growth factor or combinations thereof. In an embodiment, the immunomodulatory cytokine is IL-4 and/or IL-10 and the growth factor is transforming growth factor beta (TGF-β). In an embodiment, the small molecule, antibiotic, immunomodulatory cytokine growth factor or combinations thereof is encapsulated in the nano/microparticles, wherein the encapsulated nano/microparticles are dispersed within a biopolymer. In one embodiment, the biopolymer comprises alginate and calcium chloride. In an embodiment, the alginate is a monodisperse heparin-functionalized alginate. In an embodiment, the biopolymer comprises heparin-functionalized mesoporous silica microparticles. In one embodiment, the monodisperse heparin-functionalized alginate is an encapsulated alginate-heparin microparticle. In some embodiments, the encapsulated alginate-heparin microparticle is dispersed in a second biopolymer. In an embodiment, the second biopolymer is alginate, GelMA or a combination thereof. In a particular embodiment, the immunomodulatory cytokine and the growth factor reprogram M1 and M0 macrophages toward M2 macrophages. In some embodiments, the sustained release of the second drug is a release of from 2 weeks to 8 weeks. In one embodiment, the sustained release of the second drug is a release of from 2 weeks to 4 weeks. In some embodiments, the drug-loaded transdermal microneedles have a height ranging from about 300 μm to about 1200 μm. In one embodiment, the drug-loaded transdermal microneedles comprise a base diameter of from about 100 μm to about 400 μm. In an embodiment, the drug-loaded transdermal microneedles have an aspect ratio of height to diameter of about 5 or more. In an embodiment, the plurality of drug-loaded transdermal microneedles comprises from 10 to 90 transdermal microneedles. In an embodiment, the plurality of drug-loaded transdermal microneedles comprises from 100 to 900 transdermal microneedles. In a particular embodiment, the transdermal microneedles loaded with the second drug comprise an antibiotic, antibacterial agent, an anti-inflammatory agent, a growth factor, a cytokine, or any combination thereof. In one embodiment, the second drug in the drug-loaded transdermal microneedles is soluble, is in nanoparticles, is in microparticles, or any combination thereof. In an embodiment, the first drug in the detachable support layer comprises an antibiotic, antibacterial agent, an anti-inflammatory agent, a growth factor, a cytokine or any combination thereof. In some embodiments, the first drug in the detachable support layer is soluble, is in nanoparticles is in microparticles, or any combination thereof. In a particular embodiment, the first drug and/or the second drug is coated on the microneedles, the first drug and/or the second drug is coated on the detachable support, or both, and the first drug and/or the second are independently selected from an antibiotic, antibacterial agent, an anti-inflammatory agent, a growth factor, a cytokine or any combination thereof. In an embodiment, the first drug and/or the second drug is in nanoparticles or in microparticles. In one embodiment, the second drug in the drug-loaded transdermal microneedles, is independently an antibiotic, antibacterial agent, an anti-inflammatory agent, a growth factor, a cytokine or any combination thereof. In one embodiment, the second drug loaded in the transdermal microneedles comprises an antibiotic, an anti-inflammatory agent, a cytokine, a growth factor, or a combination thereof, and the detachable support layer comprises an antibiotic. In an embodiment, the second drug loaded in the transdermal microneedles comprises microparticles comprising an anti-inflammatory agent, a cytokine, a growth factor, or a combination thereof, and the detachable support layer comprises an antibiotic. In some embodiments, the second drug loaded in the transdermal microneedles comprises an anti-inflammatory agent, and the detachable support comprises an antibiotic. In an embodiment, the second drug loaded in the transdermal microneedles comprise a cytokine, and the detachable support comprises an antibiotic. In a particular embodiment, the antibiotic or antibacterial agent is tetracycline, doxycycline, chlorhexidine and/or minocycline. In an embodiment, the periodontal drug delivery system comprises an amount of from about 50 ng up to about 1,500 ng of the antibacterial agent per transdermal microneedle patch. In an embodiment of the periodontal drug delivery system, the anti-inflammatory agent is a steroid and/or a non-steroidal anti-inflammatory drug. In one embodiment, the steroid is dexamethasone hydrocortisone, cortisone, and/or prednisone. In an embodiment, the non-steroidal anti-inflammatory drug is indomethacin, naproxen, ibuprofen, flurbiprofen and/or piroxicam. In some embodiments, the periodontal drug delivery system is administered to the subject, wherein the diseased periodontal tissue comprises periodontal ligament, cementum, gingiva, and/or alveolar bone. In one embodiment, the subject has periodontitis and/or gingivitis.

Other features and advantages of this invention will become apparent from the following detailed description, examples and figures. It should be understood, however, that the detailed description and specific examples while indicating certain embodiments of the invention are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure, the inventions of which can be better understood by reference to one or more of these drawings in combination with the detailed description of specific embodiments presented herein.

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIGS. 1A and 1B depict schematically the microneedle patches embodied herein. FIG. 1A is a schematic of the transdermal administration of microneedle patches to treat periodontitis and gingivitis. Micron-sized needles will be detached from the support, stay in gingival tissue, release antibiotics and/or other drugs, and then degrade. FIG. 1B shows in-pocket administration of biodegradable microneedle patch that will provide structural stability via swelling and release of antibiotics, anti-inflammatories, cytokines or growth factors locally.

FIGS. 2A, 2B and 2C describe the making and testing of the microneedle patch. FIG. 2A is a schematic representation of microfluidic used for hydrodynamic flow focusing of hydrophobically-modified chitosan stream using sheath flow of basic water. FIG. 2B depicts the assessment of compressive force (left) and characteristics of three polymers. FIG. 2C depicts the release of tetracycline from a chitosan-heparin microneedle patch.

FIG. 3 depicts the microneedle patch fabrication procedure. FIG. 3A shows drugs (soluble or encapsulated in nanoparticles) are be mixed with polymer 1 and then cast over the mold, such that after the formatting of microneedles, the drugs are encapsulated. The polymer 2 as a support layer will cast over the mold. FIG. 3B shows drugs (soluble) will be mixed with polymer 2 and then cast over the mold that already has formed microneedles, thus forming a support layer which is drug-loaded. There are no nano-/microparticles included in the support layer. The support layer (based on gelatin or PVP) will dissolve quickly in the body, the drug (antibiotics) will be released in a burst. FIG. 3C shows protein therapeutics (e.g., cytokine) encapsulated into microparticles and then mixed with polymer 1 during the casting over the mold; thus, after the formatting of microneedles, the drugs are encapsulated. The polymer 2 as a support layer will cast over the mold.

FIGS. 4A, 4B, 4C, 4D, 4E and 4F depict the structure and characteristics of microneedles. FIG. 4A is a photograph of 11×11 array of microneedles. The needles are made of Alginate (1.5% w/v) stained with trypan blue (2% v/v), whereas the base is made of gelatin (10% w/v) that can liquefy in physiological temperature. FIG. 4B is a scanning electron micrographs of designed microneedles. FIG. 4C shows the microneedles geometry and dimension can be engineered. FIG. 4D shows the mechanical behavior of microneedles, in FIG. 4E with and without PLGA nanoparticles. FIG. 4F shows the degradation profile of alginate-based microneedles in PBS and human saliva at 37° C.

FIG. 5 depicts a microneedles patch penetrated on freshly harvested porcine jaw (middle and left panels). The gum also extracted and kept in 37° C. incubator after the administration of the patch for 30 minutes to monitor gelatin dissolution (right panel).

FIG. 6 shows the cumulative in vitro release of tetracycline from different formulations of microfluidic synthesized as well as traditional NPs at 37° C. and pH 7.4.

FIG. 7 shows the antibacterial effect of designed patches against P.g. Full patch includes tetracycline-loaded gelatin support and tetracycline-loaded PLGA NPs in microneedles.

FIG. 8 shows the change in mechanical properties of alginate-based microneedles after 1 or 5 times of x-ray irradiation at 25 kGy dose compared to freshly prepared samples.

FIGS. 9A, 9B, 9C and 9D describe protein containing microparticles. FIG. 9A depicts surface chemistry and FIG. 9B a SEM image of synthesized mesoporous microparticles. FIG. 9C shows the degree of heparin-conjugation of silica particles with various initial amounts of heparin in the reaction mixture. FIG. 9D shows the binding efficiency of TGF-β and interleukin-4 to the microparticles.

FIGS. 10A, 10B and 10C show cytokine delivery by the microneedle patch. FIG. 10A is a schematic of the microneedle patch fabrication for local delivery of cytokines. FIG. 10B shows a SEM of silica-heparin microparticle loaded alginate microneedles. Note: The microneedles' tips removed using focused ion beam for better visualization of encapsulated microparticles. FIG. 10C shows a significant (>7 fold) improvement in mechanical properties of microneedles upon encapsulation of silica-based microparticles.

FIGS. 11A, 11B, 11C and 11D show the immunomodulatory activity of the microneedle patch. FIG. 11A depicts the sustained release of TGF-β mediates development of induced regulatory T-cells (iT-reg cells). FIG. 11B shows cumulative in vitro release of TGF-β and interleukin-4 from silica-heparin microparticles at 37° C. and pH 7.4. FIG. 11C shows a flow cytometric analysis of iT-reg development was assessed and judged by Foxp3 and CD25 co-expression after coculture of naïve CD4+ T-cells with anti-CD3 and anti-CD28 for 4 days. The indicated range of TGF-β concentrations was applied either in a soluble format or via microparticles at the same time as activation signal. FIG. 11D depicts the change in expression of surface markers and cytokines after treatment of macrophages with IL-4 as assessed using qPCR.

FIGS. 12A-12B show proposed research. (FIG. 12A) Schematic of the in-pocket transgingival administration of microneedle patches to treat periodontitis. Micron-sized needles will be detached from the membrane support upon support dissolution, stay in periodontal tissues, release antibiotics/cytokines locally, and then degrade. (FIG. 12B) Schematic of research aims: (1) fabricate microneedle patch and test antibiotic delivery; (2) investigate the effect of immunomodulatory cytokine delivery; and (3) evaluate the in vivo therapeutic outcome.

FIG. 13 shows a schematic of the microneedle patch fabrication procedure.

FIGS. 14A-14F show preparation of microneedles. FIG. 14A is a photograph of 11×11 array of microneedles. The needles are made of Alginate (1.5% w/v) stained with trypan blue (2% v/v), whereas the base is made of gelatin (10% w/v) that can liquify in physiological temperatures. (FIG. 14B) Scanning electron micrographs of designed microneedles. (FIG. 14C) Microneedle geometry and dimension can be engineered. (FIG. 14D) Mechanical behavior of microneedles (FIG. 14E) with and without PLGA nanoparticles. (FIG. 14F) Degradation profile of alginate-based microneedles in PBS and human saliva at 37° C.

FIGS. 15A-15B show microneedle patch penetrated on freshly harvested porcine gingiva. Gingiva was extracted and kept in 37° C. after patch administration for 30 minutes to monitor gelatin dissolution prior to imaging for (14A) trypan blue or (14B) rhodamine.

FIG. 16 shows cumulative in vitro release of tetracycline from different formulations of microfluidic and traditionally synthesized NPs at 37° C. and pH 7.4.

FIG. 17 shows Antibacterial effect of designed patches against P.g. Full patch includes tetracycline-loaded gelatin support and tetracycline-loaded PLGA NPs in microneedles.

FIG. 18 shows change in mechanical properties of alginate microneedles after 1 or 5 cycles of x-ray irradiation at 25 kGy dose compared to freshly prepared samples.

FIG. 19 shows cytokine regulation of macrophages and T cells. The release of IL-4 and TGF-beta from microneedles inside periodontal tissues will alter these responses to control the infection, inflammation, and promote regeneration of the periodontium.

FIGS. 20A-20B show heparin-based conjugates. (20A) Degree of heparin-conjugation of alginate with various initial amounts of heparin in the reaction mixture. Inset: Bright-field image of synthesized microparticles. (20B) Binding efficiency of TGF-beta and IL-4 to microparticles (mean±SD; n=5, **p<0.01).

FIGS. 21A-21C show successful encapsulation of cytokine-loaded alginate particles into alginate microneedles (21A) Schematic of the microneedle patch fabrication for local delivery of cytokines. (21B) SEM of microparticle loaded alginate microneedles. Note: Microneedle tips are removed using focused ion beams for better visualization of encapsulated microparticles. (21C) Significance (>7 fold) improvement in microneedle mechanical properties upon microparticles encapsulation due to their more compact structures.

FIGS. 22A-22B show results of prolonged release of IL-4 and TGF-β from designed platforms (22A) Sustained TGF-β release mediates development of induced regulatory T-cells (iT-reg cells). (22B) Cumulative in vitro release of TGF-β and IL-4 from alginate-heparin microparticles at 37° C. and pH 7.4.

FIGS. 23A-23B show successful reprograming of M1 (and M0) macrophages toward M2 upon treatment with IL-4. (23A) mRNA expression of pro-inflammatory and (23B) anti-inflammatory genes for plain microneedle versus microneedle with IL-4 loaded silica-heparin particles (mean±SD; n=5, **p<0.01, ***p<0.001).

FIG. 24 shows flow cytometric analysis of iT-reg development was assessed by Foxp3 and CD25 co-expression after co-culture of naïve CD4+ T-cells with anti-CD3 and anti-CD28 for 4 days in the presence of TGF-β and IL-2 releasing silica-heparin microparticles. (mean±SD; n=5, **p<0.01, ***p<0.001).

FIG. 25 shows UV irradiation did not cause damage to protein stability and its function as the microneedles patch demonstrated the same effectiveness.

FIGS. 26A-26D is data that shows periodontal tissue regeneration in rat animal model. (26A) Microcomputed tomography (μCT) analyses of the rat maxilla representing the control (healthy), the defect site with blank and therapeutic (combinatorial: microneedle patches containing both tetracycline- and cytokine-loaded particles) microneedle patch treatment. All specimens were normalized, and μCT images were calibrated for to enable quantitative comparisons. Tetracycline: 0.5 mg; IL-4: 40 ng and TGF-β: 40 ng per patch. (26B) Quantitative analyses of vertical bone recovery as determined by measuring the distance between the bone crest and cementoenamel junction (CEJ) before and after treatment. (26C) The relative volumetric bone recovery was calculated using 3D reconstructed volumes. (26D) Quantification of inflammatory (TNF-α and IL-1β) cytokines 4 weeks post microneedle implantation (n=3). Presented data are expressed as mean±SD. The results were statistically analyzed using one-way ANOVA with post-hoc analysis. p<0.01 for therapeutic vs. blank patches.

FIGS. 27A-27B show in vivo biocompatibility of alginate. (27A) Immunostaining for macrophage (CD68) and lymphocyte (CD3) after subcutaneous implantation in rats (asterisks indicate alginate parts) for 7 days. Nuclei were stained with DAPI. (27B) Hematoxylin/eosin staining showing the subconscious degradation.

FIGS. 28A-28B show in vivo cytocompatibility of microneedles. Whole-blood analysis of minipigs after insertion of various microneedle formulations. Values were compared to those for pigs without any treatments and those without any therapeutic (antibiotic/IL-4/TGF-beta) inserted (Control Microneedles). White blood cells: WBC (White blood cells), NE (Neutrophils), LY (Lymphocytes), M0 (Monocytes), EO (Eosinophils), BA (Basophils). Red blood cells: HCT (Hematocrit), RBC (Red blood cells), HB (Hemoglobin), MCV (Mean corpuscular volume), MCH (Mean corpuscular hemoglobin), MCHC (Mean corpuscular hemoglobin concentration), Platelets: PLT (Platelet count). Comprehensive metabolic screening of minipigs after insertion of various microneedle formulations as indicated. Values were compared to those for pigs without any treatments and those without any therapeutic (antibiotic/IL-4/TGF-beta) inserted (Control Microneedles). Liver function assessment: ALT (alanine aminotransferase), AST (aspartate aminotransferase), BUN (blood urea nitrogen), LDH (Lactate dehydrogenase). Kidney function assessment: CREAT (Creatinine), GLU (Glucose). Electrolytes: Calcium (CA), CO2 (carbon dioxide), MG (Magnesium), and PHOS (Potassium).

FIGS. 29A-29C show effects of drug delivery on bone regeneration. (29A) Ligature induced periodontal disease model in pigs. (B) Mucoperiosteal flaps were elevated, uncovering the alveolar bone adjacent to the lingual aspect of the 3^{rd} and 4^{th} premolars, and first maxillary molar to place microneedle-based patches at the diseased sites. (29B) CT images of the pig maxilla showing control (healthy) and ligature-induced periodontal disease (29C). Yellow arrows point to periodontal bone loss after disease induction.

### DETAILED DESCRIPTION OF THE INVENTION

The present subject matter may be understood more readily by reference to the following detailed description which forms a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In the present disclosure, the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment includes from the one particular and/or to the other particular value.

Similarly, when values are expressed as approximations, by use of the antecedent “about,” it is understood that the particular value forms another embodiment. All ranges are inclusive and combinable. In the context of the present disclosure, by “about” a certain amount it is meant that the amount is within ±20% of the stated amount, or preferably within ±10% of the stated amount, or more preferably within ±5% of the stated amount.

As used herein, the terms “treat”, “treatment”, or “therapy” (as well as different forms thereof) refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. Those in need of treatment include those already with the disease or condition as well as those prone to having the disease or condition or those in which the disease or condition is to be prevented.

As used herein, the terms “component,” “composition,” “formulation”, “composition of compounds,” “compound,” “drug,” “pharmacologically active agent,” “active agent,” “therapeutic,” “therapy,” “treatment,” or “medicament,” are used interchangeably herein, as context dictates, to refer to a compound or compounds or composition of matter which, when administered to a subject (human or animal) induces a desired pharmacological and/or physiologic effect by local and/or systemic action. A personalized composition or method refers to a product or use of the product in a regimen tailored or individualized to meet specific needs identified or contemplated in the subject.

The terms “subject,” “individual,” and “patient” are used interchangeably herein, and refer to an animal, for example a human, to whom treatment with a composition or formulation in accordance with the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys. The compositions described herein can be used to treat any suitable mammal, including primates, such as monkeys and humans, horses, cows, cats, dogs, rabbits, and rodents such as rats and mice. In one embodiment, the mammal to be treated is human. The human can be any human of any age. In an embodiment, the human is an adult. In another embodiment, the human is a child. The human can be male, female, pregnant, middle-aged, adolescent, or elderly. According to any of the methods of the present invention and in one embodiment, the subject is human. In another embodiment, the subject is a non-human primate. In another embodiment, the subject is murine, which in one embodiment is a mouse, and, in another embodiment is a rat. In another embodiment, the subject is canine, feline, bovine, equine, laprine or porcine. In another embodiment, the subject is mammalian.

Conditions and disorders in a subject for which a particular drug, compound, composition, formulation (or combination thereof) is said herein to be “indicated” are not restricted to conditions and disorders for which that drug or compound or composition or formulation has been expressly approved by a regulatory authority, but also include other conditions and disorders known or reasonably believed by a physician or other health or nutritional practitioner to be amenable to treatment with that drug or compound or composition or formulation or combination thereof.

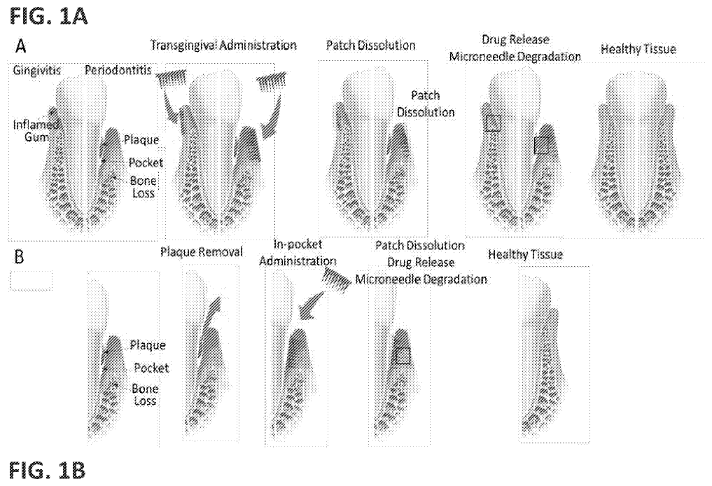

To address the clinical need for periodontal treatment and regeneration, the inventors engineered a delivery patch of microneedles as a tunable platform with the ability to inhibit bacteria, control inflammation, and direct regeneration of periodontium-like tissues. The aim of this project was to achieve precise spatiotemporal control of sequential antibiotics and immunomodulatory cytokine delivery that can regulate the microenvironment to effectively manage bacterial infection, control periodontitis progression, and reverse periodontal/gingival tissue degeneration. Here, the inventors developed and optimized periodontal patches including a detachable membrane support and microneedles containing nano/microparticles for drug delivery (FIG. 1). The patch is applied to periodontal tissues: the membrane support will dissolve quickly for a burst release of antibiotics to immediately reduce bacterial content in the microenvironment, and the biocompatible and bioresorbable microneedles will penetrate and stay in the gingival tissues for a sustained release of antibiotics and cytokines. This creative delivery platform is in the periodontal tissues, is suture-free and tunable, and offers a minimally-invasive therapeutic modality to effectively treat periodontitis. Although microneedles have been widely explored for transdermal drug delivery and demonstrated scalability and cost-effective manufacturing for applications such as cosmetic skin care and vaccine injection, microneedle drug delivery for immune cell modulation has not been tested for periodontal treatment. The studies described herein provide unprecedented insight into both the technology feasibility and scientific approach, which will have broad impact on periodontal disease treatment and the regeneration of many other tissues. To make the co-delivery system a universal technology platform for the delivery of a variety of drugs, we used nano/microparticle encapsulation to customize drug loading (small molecules, proteins etc.) in a microfluidic device, where various drug-loaded microparticles can be easily embedded into a microneedle array during fabrication. This microneedle drug delivery system is a highly versatile technology platform to deliver various drugs, as well as many other applications. For example, this proposed microneedle patch is surgically placed inside the periodontal pocket by a dentist or specialist; however, these patches can also be further developed as an over-the-counter product for transgingival self-administered delivery of antibiotics to treat gingivitis and mild periodontitis. In this way, patients may apply the patches to easily accessible and inflamed gingival tissues for home self-care between dental visits, or in circumstances where access to care is limited. This has become increasingly relevant during the current COVID-19 pandemic where patients were unable to undergo routine dental maintenance visits. In addition, this microneedle device can be co-delivered with other dental biomedical devices or biosensors to decrease infection or inflammatory reactions associated with dental implants. The approach to engineer immune cell responses is novel and will have wide applications to treat oral diseases.

Described herein are methods for manufacturing a novel periodontal patch for the treatment of periodontitis by engineering a microneedle array with tunable multistage release and degradation properties. A minimally invasive delivery platform enabling the controlled delivery of antibiotics, peptides and therapeutic proteins. The overall concept, approach, and methodologies described herein represent a significant departure from previous studies. In contrast to currently available periodontal treatment, the engineered microneedle patch is a first-in-class platform which is easy-to-handle, self-adhesive, and biodegradable with modular drug release properties. The immunomodulation of macrophage polarity (and T cells), in combination with antibiotic delivery, is a novel approach for periodontal regeneration. Encapsulation of antibiotics and cytokines into nano/microparticles and their incorporation into microneedles will give the engineered microneedle patch modular properties to immediately reduce bacterial content and tune the inflammatory environment toward periodontal and alveolar bone regeneration. Furthermore, the biodegradable microneedles can act as a delivery vehicle for various small molecule drugs and therapeutic proteins. Prolonged release of therapeutic agents inside periodontal tissues through hundreds of microneedles per square centimeter will provide enhanced drug distribution over target tissues, which will overcome drug diffusion limits and reduce the therapeutic dose. This novel microneedle patch can be simply handled and placed at the diseased/defect site, easily integrated into periodontal tissues, and have a tunable degradation rate for optimal repair and regeneration. The microneedles detach from the supporting patch, do not need sutures to stay in place, and do not cause discomfort. The developed platform utilizes objective clinical and radiographic parameters for disease resolution and periodontal regeneration, that will provide significant insight for the translation of the technology into clinical applications. The research team provides proof-of-concept data to demonstrate the feasibility of the platform, and significant knowledge gained for eventual translation to clinical practice.

Also described herein is a suture-free, drug-loaded and biodegradable patch with an array of microneedles for the treatment of periodontitis that can regulate the inflammatory microenvironment to supress bacterial growth and tune the local immunogenic microenvironment to control inflammation and accelerate periodontal regeneration. The patch's physicochemical properties, including geometry, shape, mechanical properties, degradation, and two-phase antibiotic loading/release was optimized to provide strong antibacterial properties. (Example 11). The microneedles in the patch are loaded with microparticles for sustained delivery of immunomodulatory cytokines to alter macrophage lineage (M1 to M2) and to help formation of regulatory T cells. (Example 12). The functionality of the engineered patches is confirmed in vivo using a ligature-induced periodontitis in minipigs (Example 13). The developed strategy is outlined in FIG. 1.

This invention is a patch-based drug delivery system with antibacterial and/or anti-inflammatory properties for dental applications. Such a delivery platform can manage or control periodontitis or gingivitis, and promote periodontal tissue regeneration. The patch can be applied easily near the disease or defect site, to the gingiva or into the gingival pocket. The patch contains tens to hundreds of microneedles per square centimeter that will create micron sized pores in the gingiva.

In one embodiment, the transgingival microneedles are drug-loaded solid polymeric needle-like structures. In one embodiment, the microneedles are also coated with a drug. In one embodiment the microneedles are provided on a detachable support (patch support). In one embodiment, the microneedles detach from the patch support after administration and will stay in the gingival tissue upon the removal of the patch. The detaching mechanism is based, in one embodiment, on quick dissolving of gelatin-based support at body temperature. In one embodiment, the detachable support also contains a drug that is released while the detachable support is retained at the site of application. In one embodiment, the microneedles remain in gum tissue for multiple days or weeks after application. The residence time of the detachable support and the microneedles can be adjusted by adjusting the compositions of these components. As will be described further below, the drug in the microneedles, the drug coated on the microneedles and the drug in the detachable support may be the same or different, may be soluble or in the form of microparticles, nanoparticles or other controlled release forms, and may be an antimicrobial agent, an anti-inflammatory agent, a cytokine, a growth factor, or any other therapeutic agent useful in the treatment of gingivitis or periodontal disease. In one embodiment a drug is present in the microneedles, and is optionally present as a coating on the microneedles or in the detachable support, or both. As noted above, the drug for each location is independently selected.

In one embodiment, microneedle-mediated delivery improves patient compliance as its administration does not stimulate nerves or cause pain. Sustained and prolonged release of various antibacterial agents (e.g., tetracycline, doxycycline, minocycline), anti-inflammatory agents (e.g., dexamethasone), growth factors and/or cytokines from microneedles can, in one embodiment, reduce local inflammation and in another embodiment, promote regeneration of lost bone.

In one embodiment, microneedles have proper mechanical properties and degradation rates to deliver their cargo up to and over two weeks (2-4 weeks) for periodontal tissue repair.

These microneedle-based patches are fabricated, and the material composition and dimensions are optimized for the desired use. In one embodiment, the loading and release profile of an antibiotic such as tetracycline hydrochloride are optimized and tuned based on severity of disease and required dose. Density and dimension of microneedles can be tuned based on the specific application. In one embodiment, approximately 250 ng of tetracycline can be delivered per microneedle. Considering local and intra-gingival presentation of these molecules, the designed microneedle patch is optimized to carry enough drug to deliver the total needed payload. In addition, the microneedle concentration can be increased and the functionality tested in periodontal defect in animal model. In one embodiment, a well-established periodontal defect model in rats is used.

An optimized periodontal patch based on microneedles is described comprising microneedles placed in gingival tissues can be dispensed and stay at the target site for the course of treatment. The patch does not need adhesive or suture for sealing. Instead, these patches are intended to be absorbed by the body for short term use (<30 days).

The terms “micro-patch” and “microneedle patch” are synonymous. The terms “detachable support” and “support layer” and “patch support” and “detachable support layer” are used interchangeably. The micro-patch comprises microneedles and a detachable support.

As will be shown in the examples below, successful fabrication of the immunomodulatory patch with tunable characteristics was demonstrated. By changing the type and concentration of loaded cytokine concentration and heparin conjugation density, the polarization of macrophages and formation of regulatory T cells can be tuned in vitro. The micro-patch platform can be expanded to load and deliver wide variety of therapeutic proteins. Fabrication of a removable microneedle patches that can control tetracycline release to fight bacterial infection was demonstrated. By changing polymer composition and drug concentrations as well as changing microfabricated mold architecture, the delivery platform may be optimized to effectively penetrate gingival tissue and release its cargo at target sites. An initial burst release of tetracycline, followed by its sustained release over 2-4 weeks, initially treats the bacterial infection then over time reduces bacterial content. The polymer type and its properties (e.g., molecular weight, crosslinking density) were optimized to provide prolonged release profile, which is advantageous for the proposed application. The compressive strength of the alginate needles can be increased by enhancing the calcium and polymer concentrations. No significant change in properties of patches and antibiotic was observed after the radiation sterilization process. The degradation rate of alginate was found to be between 4 to 8 weeks in vivo. High cell viability (>85%) is verified on exposure to the engineered microneedles. No significant foreign body response to the implanted drug-free microneedles was observed. The micro-patch offers significant benefit in the treatment of highly prevalent dental diseases responsible for significant worldwide morbidity.

Diseases that can be treated by the micro-patch include diseased periodontal tissue such as but are not limited to periodontal ligament, cementum, gingiva, and/or alveolar bone. The subject has periodontitis and/or gingivitis. The subject is a mammalian subject, preferably a human, although the micro-patch and methods described herein may also be used for dental disease of companion, zoo, and livestock animals, among others.

As described herein, a periodontal drug delivery system is provided comprising a microneedle patch, the microneedle patch comprising a detachable support layer and a plurality of detachable, drug-loaded microneedles thereon. As will be described below, the detachable support layer has the function to hold and deliver the microneedles to the periodontal site, after which the detachable support layer detaches from the microneedles, leaving the microneedles embedded in the periodontal tissue to release their drug content (in the microneedles, on the microneedles, or both). The detachable support layer is fabricated typically as a flat sheet such that the microneedles are projecting perpendicularly from one flat side of the detachable support layer. In one embodiment, the microneedle patch is pressed into the periodontal tissue such that the microneedles penetrate the tissue, and the detachable support layer is sealed against the periodontal tissue. By biodegradation or other detachment, the detachable support layer detaches or separates from the microneedles and the gum surface, leaving the microneedles in place. The detachment may be rapid, or prolonged, depending on the components of the detachable support layer and its desired purpose. For the purpose of merely placing the drug-loaded microneedles into the periodontal tissue, detachment may be rapid and thus a rapidly biodegradable material may be used for the detachable support layer. Such a support layer may comprise a coating of a drug for a burst of rapid release during placement of the microneedle patch. In other embodiments, a prolonged duration of the detachable support layer sealed to the periodontal tissue may permit a drug in the detachable support layer to be delivered to the periodontal tissue or into the periodontal pocket, for therapeutic benefit in addition to release of drug by the microneedles. In some embodiments, the kinetics of release of the drugs independently selected and located in and/or on the microneedles or detachable support layer are provided to treat the condition or disease in a safe and effective manner, and in one embodiment to reduce or prevent its recurrence.

The microneedles are typically arranged on one flat side of the detachable support layer as an array or pattern, in one embodiment, an 11×11 array. Typically, the microneedles have an elongated, conical shape wherein each microneedle has its base disposed on the detachable support layer, and the opposite, tapered/pointed end, is pointed away from the surface, such that the microneedles are perpendicular to the surface and parallel to each other. Thus, the array of microneedles can be readily inserted into the tissue, the array of pointed ends inserted into the tissue until the detachable support layer is sealed to the periodontal surface. In one embodiment, the microneedle patch is pushed against the periodontal surface in the direction to insert the needles perpendicular to the tissue surface. In other embodiments, the microneedles are parallel to each other but may be angled from the detachable support layer less than a right angle. In one embodiment, for ease in insertion into the gingival tissue within a gingival cavity, the microneedle patch comprises microneedles that are angled 45 degrees from the surface, such that insertion into the periodontal tissue can be achieved by applying a lateral force to the detachable support layer. In other embodiments, the angle is between 45 degrees and 90 degrees. Variations in the design and fabrication of the microneedle patch will be provided to suit various purposes, sites of location, different gingival pocket configurations, gum and tooth sizes among the patient population, etc., without deviating from the spirit of the invention.

The size of the detachable support layer may be guided by the area encompassed by the array of microneedles. At minimum, the one side of the detachable support layer is decorated entirely with microneedles. In one embodiment, a border of undecorated support layer surrounds the microneedle array on four sides, on three sides, on two sides, or on one side. The portion of the support layer not decorated with microneedles is capable of sealable application to the periodontal tissue. Thus, the support layer between the microneedle bases and the border around the array, if any, can be applied to the periodontal tissue.

The number of microneedles, the layout of the microneedle array, the distance between microneedles, the amount of border around one or more sides of the array, are governed by the intended location and use of the periodontal delivery system. In some embodiments, the microneedle patch's detachable support layer is square, rectangular, oval, round, triangular, or any other shape to be conducive to a site of application and for the intended use. In one embodiment, a microneedle patch can be cut to suit the particular location, and number of microneedles to deliver an intended amount of one or more drugs.

In one aspect, provided herein is a periodontal drug delivery system comprising a microneedle patch, the microneedle patch comprising: (a) a detachable support layer comprising a surface capable of a sealable application to a diseased periodontal tissue of a subject in need thereof, wherein the detachable support layer comprises a first drug, and the detachable support layer is formulated for immediate release of the first drug; and (b) a plurality of detachable, transdermal microneedles having bases disposed on the surface of the detachable support layer and projecting perpendicularly therefrom, wherein the transdermal microneedles comprise nano/microparticles loaded with a second drug, and the transdermal microneedles are formulated for sustained release of the second drug.

In an embodiment, the detachable support layer, the transdermal microneedles, or both, independently comprise a biodegradable polymer. In some embodiments of the periodontal drug delivery system the biodegradable polymer is independently selected from the group consisting of chitosan, alginate, gelatin, hyaluronic acid (HA), polycaprolactone (PCL), poly(lactic-co-glycolic acid) (PLGA), hydrophobically-modified alginate, hydrophobically-modified chitosan, alginate-heparin, chitosan-heparin, methacrylated gelatin (GelMA), and combinations thereof. In an embodiment, the biodegradable polymer dissolves at body temperature, the body temperature ranging from 97° F. (36.1° C.) to 99° F. (37.2° C.). In an embodiment, the detachable support layer is non-membranous and non-adhesive. In certain embodiments, the detachable support layer, the microneedles, or both are porous. In a particular embodiment, the first drug is an antibiotic or an antibacterial agent. In an embodiment, the first drug is present within the detachable support layer, the first drug is coated on the detachable support layer, or a combination thereof. In some embodiments, the first drug is dispersed within a biodegradable polymer. In an embodiment, the nano/microparticles loaded with the second drug are dispersed within a biodegradable polymer. In a particular embodiment, the second drug is the same drug as the first drug, is a different drug than the first drug, or combinations thereof. In some embodiments, the different drug is a small molecule, an antibiotic, an immunomodulatory cytokine, a growth factor or combinations thereof. In an embodiment, the immunomodulatory cytokine is IL-4 and/or IL-10 and the growth factor is transforming growth factor beta (TGF-β). In an embodiment, the small molecule, antibiotic, immunomodulatory cytokine growth factor or combinations thereof is encapsulated in the nano/microparticles, wherein the encapsulated nano/microparticles are dispersed within a biopolymer. In one embodiment, the biopolymer comprises alginate and calcium chloride. In an embodiment, the alginate is a monodisperse heparin-functionalized alginate. In an embodiment, the biopolymer comprises heparin-functionalized mesoporous silica microparticles. In one embodiment, the monodisperse heparin-functionalized alginate is an encapsulated alginate-heparin microparticle. In some embodiments, the encapsulated alginate-heparin microparticle is dispersed in a second biopolymer. In an embodiment, the second biopolymer is alginate, GelMA or a combination thereof. In a particular embodiment, the immunomodulatory cytokine and the growth factor reprogram M1 and M0 macrophages toward M2 macrophages. In some embodiments, the sustained release of the second drug is a release of from 2 weeks to 8 weeks. In one embodiment, the sustained release of the second drug is a release of from 2 weeks to 4 weeks. In some embodiments, the drug-loaded transdermal microneedles have a height ranging from about 300 μm to about 1200 μm. In one embodiment, the drug-loaded transdermal microneedles comprise a base diameter of from about 100 μm to about 400 μm. In an embodiment, the drug-loaded transdermal microneedles have an aspect ratio of height to diameter of about 5 or more. In an embodiment, the plurality of drug-loaded transdermal microneedles comprises from 10 to 90 transdermal microneedles. In an embodiment, the plurality of drug-loaded transdermal microneedles comprises from 100 to 900 transdermal microneedles. In a particular embodiment, the transdermal microneedles loaded with the second drug comprise an antibiotic, antibacterial agent, an anti-inflammatory agent, a growth factor, a cytokine, or any combination thereof. In one embodiment, the second drug in the drug-loaded transdermal microneedles is soluble, is in nanoparticles, is in microparticles, or any combination thereof. In an embodiment, the first drug in the detachable support layer comprises an antibiotic, antibacterial agent, an anti-inflammatory agent, a growth factor, a cytokine or any combination thereof. In some embodiments, the first drug in the detachable support layer is soluble, is in nanoparticles is in microparticles, or any combination thereof. In a particular embodiment, the first drug and/or the second drug is coated on the microneedles, the first drug and/or the second drug is coated on the detachable support, or both, and the first drug and/or the second are independently selected from an antibiotic, antibacterial agent, an anti-inflammatory agent, a growth factor, a cytokine or any combination thereof. In an embodiment, the first drug and/or the second drug is in nanoparticles or in microparticles. In one embodiment, the second drug in the drug-loaded transdermal microneedles, is independently an antibiotic, antibacterial agent, an anti-inflammatory agent, a growth factor, a cytokine or any combination thereof. In one embodiment, the second drug loaded in the transdermal microneedles comprises an antibiotic, an anti-inflammatory agent, a cytokine, a growth factor, or a combination thereof, and the detachable support layer comprises an antibiotic. In an embodiment, the second drug loaded in the transdermal microneedles comprises microparticles comprising an anti-inflammatory agent, a cytokine, a growth factor, or a combination thereof, and the detachable support layer comprises an antibiotic. In some embodiments, the second drug loaded in the transdermal microneedles comprises an anti-inflammatory agent, and the detachable support comprises an antibiotic. In an embodiment, the second drug loaded in the transdermal microneedles comprise a cytokine, and the detachable support comprises an antibiotic. In a particular embodiment, the antibiotic or antibacterial agent is tetracycline, doxycycline, chlorhexidine and/or minocycline. In an embodiment, the periodontal drug delivery system comprises an amount of from about 50 ng up to about 1,500 ng of the antibacterial agent per transdermal microneedle patch. In an embodiment of the periodontal drug delivery system, the anti-inflammatory agent is a steroid and/or a non-steroidal anti-inflammatory drug. In one embodiment, the steroid is dexamethasone hydrocortisone, cortisone, and/or prednisone. In an embodiment, the non-steroidal anti-inflammatory drug is indomethacin, naproxen, ibuprofen, flurbiprofen and/or piroxicam. In some embodiments, the periodontal drug delivery system is administered to the subject, wherein the diseased periodontal tissue comprises periodontal ligament, cementum, gingiva, and/or alveolar bone. In one embodiment, the subject has periodontitis and/or gingivitis.

In another aspect, provided herein is a method for regenerating periodontal tissue in a subject in need thereof, the method comprising sealably applying to the periodontal tissue a microneedle patch, the microneedle patch comprising: (a) a detachable support layer comprising a surface capable of a sealable application to a diseased periodontal tissue of a subject in need thereof, wherein the detachable support layer comprises a first drug, and the detachable support layer is formulated for immediate release of the first drug; and (b) a plurality of detachable, transdermal microneedles having bases disposed on the surface of the detachable support layer and projecting perpendicularly therefrom, wherein the transdermal microneedles comprise nano/microparticles loaded with a second drug, and the transdermal microneedles are formulated for sustained release of the second drug.

In an embodiment, the detachable support layer, the transdermal microneedles, or both, independently comprise a biodegradable polymer. In some embodiments of the periodontal drug delivery system the biodegradable polymer is independently selected from the group consisting of chitosan, alginate, gelatin, hyaluronic acid (HA), polycaprolactone (PCL), poly(lactic-co-glycolic acid) (PLGA), hydrophobically-modified alginate, hydrophobically-modified chitosan, alginate-heparin, chitosan-heparin, methacrylated gelatin (GelMA), and combinations thereof. In an embodiment, the biodegradable polymer dissolves at body temperature, the body temperature ranging from 97° F. (36.1° C.) to 99° F. (37.2° C.). In an embodiment, the detachable support layer is non-membranous and non-adhesive. In certain embodiments, the detachable support layer, the microneedles, or both are porous. In a particular embodiment, the first drug is an antibiotic or an antibacterial agent. In an embodiment, the first drug is present within the detachable support layer, the first drug is coated on the detachable support layer, or a combination thereof. In some embodiments, the first drug is dispersed within a biodegradable polymer. In an embodiment, the nano/microparticles loaded with the second drug are dispersed within a biodegradable polymer. In a particular embodiment, the second drug is the same drug as the first drug, is a different drug than the first drug, or combinations thereof. In some embodiments, the different drug is a small molecule, an antibiotic, an immunomodulatory cytokine, a growth factor or combinations thereof. In an embodiment, the immunomodulatory cytokine is IL-4 and/or IL-10 and the growth factor is transforming growth factor beta (TGF-β). In an embodiment, the small molecule, antibiotic, immunomodulatory cytokine growth factor or combinations thereof is encapsulated in the nano/microparticles, wherein the encapsulated nano/microparticles are dispersed within a biopolymer. In one embodiment, the biopolymer comprises alginate and calcium chloride. In an embodiment, the alginate is a monodisperse heparin-functionalized alginate. In an embodiment, the biopolymer comprises heparin-functionalized mesoporous silica microparticles. In one embodiment, the monodisperse heparin-functionalized alginate is an encapsulated alginate-heparin microparticle. In some embodiments, the encapsulated alginate-heparin microparticle is dispersed in a second biopolymer. In an embodiment, the second biopolymer is alginate, GelMA or a combination thereof. In a particular embodiment, the immunomodulatory cytokine and the growth factor reprogram M1 and M0 macrophages toward M2 macrophages. In some embodiments, the sustained release of the second drug is a release of from 2 weeks to 8 weeks. In one embodiment, the sustained release of the second drug is a release of from 2 weeks to 4 weeks. In some embodiments, the drug-loaded transdermal microneedles have a height ranging from about 300 μm to about 1200 μm. In one embodiment, the drug-loaded transdermal microneedles comprise a base diameter of from about 100 μm to about 400 μm. In an embodiment, the drug-loaded transdermal microneedles have an aspect ratio of height to diameter of about 5 or more. In an embodiment, the plurality of drug-loaded transdermal microneedles comprises from 10 to 90 transdermal microneedles. In an embodiment, the plurality of drug-loaded transdermal microneedles comprises from 100 to 900 transdermal microneedles. In a particular embodiment, the transdermal microneedles loaded with the second drug comprise an antibiotic, antibacterial agent, an anti-inflammatory agent, a growth factor, a cytokine, or any combination thereof. In one embodiment, the second drug in the drug-loaded transdermal microneedles is soluble, is in nanoparticles, is in microparticles, or any combination thereof. In an embodiment, the first drug in the detachable support layer comprises an antibiotic, antibacterial agent, an anti-inflammatory agent, a growth factor, a cytokine or any combination thereof. In some embodiments, the first drug in the detachable support layer is soluble, is in nanoparticles is in microparticles, or any combination thereof. In a particular embodiment, the first drug and/or the second drug is coated on the microneedles, the first drug and/or the second drug is coated on the detachable support, or both, and the first drug and/or the second are independently selected from an antibiotic, antibacterial agent, an anti-inflammatory agent, a growth factor, a cytokine or any combination thereof. In an embodiment, the first drug and/or the second drug is in nanoparticles or in microparticles. In one embodiment, the second drug in the drug-loaded transdermal microneedles, is independently an antibiotic, antibacterial agent, an anti-inflammatory agent, a growth factor, a cytokine or any combination thereof. In one embodiment, the second drug loaded in the transdermal microneedles comprises an antibiotic, an anti-inflammatory agent, a cytokine, a growth factor, or a combination thereof, and the detachable support layer comprises an antibiotic. In an embodiment, the second drug loaded in the transdermal microneedles comprises microparticles comprising an anti-inflammatory agent, a cytokine, a growth factor, or a combination thereof, and the detachable support layer comprises an antibiotic. In some embodiments, the second drug loaded in the transdermal microneedles comprises an anti-inflammatory agent, and the detachable support comprises an antibiotic. In an embodiment, the second drug loaded in the transdermal microneedles comprise a cytokine, and the detachable support comprises an antibiotic. In a particular embodiment, the antibiotic or antibacterial agent is tetracycline, doxycycline, chlorhexidine and/or minocycline. In an embodiment, the periodontal drug delivery system comprises an amount of from about 50 ng up to about 1,500 ng of the antibacterial agent per transdermal microneedle patch. In an embodiment of the periodontal drug delivery system, the anti-inflammatory agent is a steroid and/or a non-steroidal anti-inflammatory drug. In one embodiment, the steroid is dexamethasone hydrocortisone, cortisone, and/or prednisone. In an embodiment, the non-steroidal anti-inflammatory drug is indomethacin, naproxen, ibuprofen, flurbiprofen and/or piroxicam. In some embodiments, the periodontal drug delivery system is administered to the subject, wherein the diseased periodontal tissue comprises periodontal ligament, cementum, gingiva, and/or alveolar bone. In one embodiment, the subject has periodontitis and/or gingivitis.

In one aspect, provided herein is a method for reducing local inflammation in periodontal tissue of a subject in need thereof, the method comprising sealably applying to the periodontal tissue a microneedle patch, the microneedle patch comprising: (a) a detachable support layer comprising a surface capable of a sealable application to a diseased periodontal tissue of a subject in need thereof, wherein the detachable support layer comprises a first drug, and the detachable support layer is formulated for immediate release of the first drug; and (b) a plurality of detachable, transdermal microneedles having bases disposed on the surface of the detachable support layer and projecting perpendicularly therefrom, wherein the transdermal microneedles comprise nano/microparticles loaded with a second drug, and the transdermal microneedles are formulated for sustained release of the second drug.

In an embodiment, the detachable support layer, the transdermal microneedles, or both, independently comprise a biodegradable polymer. In some embodiments of the periodontal drug delivery system the biodegradable polymer is independently selected from the group consisting of chitosan, alginate, gelatin, hyaluronic acid (HA), polycaprolactone (PCL), poly(lactic-co-glycolic acid) (PLGA), hydrophobically-modified alginate, hydrophobically-modified chitosan, alginate-heparin, chitosan-heparin, methacrylated gelatin (GelMA), and combinations thereof. In an embodiment, the biodegradable polymer dissolves at body temperature, the body temperature ranging from 97° F. (36.1° C.) to 99° F. (37.2° C.). In an embodiment, the detachable support layer is non-membranous and non-adhesive. In certain embodiments, the detachable support layer, the microneedles, or both are porous. In a particular embodiment, the first drug is an antibiotic or an antibacterial agent. In an embodiment, the first drug is present within the detachable support layer, the first drug is coated on the detachable support layer, or a combination thereof. In some embodiments, the first drug is dispersed within a biodegradable polymer. In an embodiment, the nano/microparticles loaded with the second drug are dispersed within a biodegradable polymer. In a particular embodiment, the second drug is the same drug as the first drug, is a different drug than the first drug, or combinations thereof. In some embodiments, the different drug is a small molecule, an antibiotic, an immunomodulatory cytokine, a growth factor or combinations thereof. In an embodiment, the immunomodulatory cytokine is IL-4 and/or IL-10 and the growth factor is transforming growth factor beta (TGF-β). In an embodiment, the small molecule, antibiotic, immunomodulatory cytokine growth factor or combinations thereof is encapsulated in the nano/microparticles, wherein the encapsulated nano/microparticles are dispersed within a biopolymer. In one embodiment, the biopolymer comprises alginate and calcium chloride. In an embodiment, the alginate is a monodisperse heparin-functionalized alginate. In an embodiment, the biopolymer comprises heparin-functionalized mesoporous silica microparticles. In one embodiment, the monodisperse heparin-functionalized alginate is an encapsulated alginate-heparin microparticle. In some embodiments, the encapsulated alginate-heparin microparticle is dispersed in a second biopolymer. In an embodiment, the second biopolymer is alginate, GelMA or a combination thereof. In a particular embodiment, the immunomodulatory cytokine and the growth factor reprogram M1 and M0 macrophages toward M2 macrophages. In some embodiments, the sustained release of the second drug is a release of from 2 weeks to 8 weeks. In one embodiment, the sustained release of the second drug is a release of from 2 weeks to 4 weeks. In some embodiments, the drug-loaded transdermal microneedles have a height ranging from about 300 μm to about 1200 μm. In one embodiment, the drug-loaded transdermal microneedles comprise a base diameter of from about 100 μm to about 400 μm. In an embodiment, the drug-loaded transdermal microneedles have an aspect ratio of height to diameter of about 5 or more. In an embodiment, the plurality of drug-loaded transdermal microneedles comprises from 10 to 90 transdermal microneedles. In an embodiment, the plurality of drug-loaded transdermal microneedles comprises from 100 to 900 transdermal microneedles. In a particular embodiment, the transdermal microneedles loaded with the second drug comprise an antibiotic, antibacterial agent, an anti-inflammatory agent, a growth factor, a cytokine, or any combination thereof. In one embodiment, the second drug in the drug-loaded transdermal microneedles is soluble, is in nanoparticles, is in microparticles, or any combination thereof. In an embodiment, the first drug in the detachable support layer comprises an antibiotic, antibacterial agent, an anti-inflammatory agent, a growth factor, a cytokine or any combination thereof. In some embodiments, the first drug in the detachable support layer is soluble, is in nanoparticles is in microparticles, or any combination thereof. In a particular embodiment, the first drug and/or the second drug is coated on the microneedles, the first drug and/or the second drug is coated on the detachable support, or both, and the first drug and/or the second are independently selected from an antibiotic, antibacterial agent, an anti-inflammatory agent, a growth factor, a cytokine or any combination thereof. In an embodiment, the first drug and/or the second drug is in nanoparticles or in microparticles. In one embodiment, the second drug in the drug-loaded transdermal microneedles, is independently an antibiotic, antibacterial agent, an anti-inflammatory agent, a growth factor, a cytokine or any combination thereof. In one embodiment, the second drug loaded in the transdermal microneedles comprises an antibiotic, an anti-inflammatory agent, a cytokine, a growth factor, or a combination thereof, and the detachable support layer comprises an antibiotic. In an embodiment, the second drug loaded in the transdermal microneedles comprises microparticles comprising an anti-inflammatory agent, a cytokine, a growth factor, or a combination thereof, and the detachable support layer comprises an antibiotic. In some embodiments, the second drug loaded in the transdermal microneedles comprises an anti-inflammatory agent, and the detachable support comprises an antibiotic. In an embodiment, the second drug loaded in the transdermal microneedles comprise a cytokine, and the detachable support comprises an antibiotic. In a particular embodiment, the antibiotic or antibacterial agent is tetracycline, doxycycline, chlorhexidine and/or minocycline. In an embodiment, the periodontal drug delivery system comprises an amount of from about 50 ng up to about 1,500 ng of the antibacterial agent per transdermal microneedle patch. In an embodiment of the periodontal drug delivery system, the anti-inflammatory agent is a steroid and/or a non-steroidal anti-inflammatory drug. In one embodiment, the steroid is dexamethasone hydrocortisone, cortisone, and/or prednisone. In an embodiment, the non-steroidal anti-inflammatory drug is indomethacin, naproxen, ibuprofen, flurbiprofen and/or piroxicam. In some embodiments, the periodontal drug delivery system is administered to the subject, wherein the diseased periodontal tissue comprises periodontal ligament, cementum, gingiva, and/or alveolar bone. In one embodiment, the subject has periodontitis and/or gingivitis.

In another aspect, provided herein is a method for promoting regeneration of bone loss in periodontal tissue of a subject in need thereof, the method comprising sealably applying to the periodontal tissue a microneedle patch, the microneedle patch comprising: (a) a detachable support layer comprising a surface capable of a sealable application to a diseased periodontal tissue of a subject in need thereof; and (b) a plurality of detachable, drug-loaded transdermal microneedles having bases disposed on the surface of the detachable support layer and projecting perpendicularly therefrom.

In an embodiment, the detachable support layer, the transdermal microneedles, or both, independently comprise a biodegradable polymer. In some embodiments of the periodontal drug delivery system the biodegradable polymer is independently selected from the group consisting of chitosan, alginate, gelatin, hyaluronic acid (HA), polycaprolactone (PCL), poly(lactic-co-glycolic acid) (PLGA), hydrophobically-modified alginate, hydrophobically-modified chitosan, alginate-heparin, chitosan-heparin, methacrylated gelatin (GelMA), and combinations thereof. In an embodiment, the biodegradable polymer dissolves at body temperature, the body temperature ranging from 97° F. (36.1° C.) to 99° F. (37.2° C.). In an embodiment, the detachable support layer is non-membranous and non-adhesive. In certain embodiments, the detachable support layer, the microneedles, or both are porous. In a particular embodiment, the first drug is an antibiotic or an antibacterial agent. In an embodiment, the first drug is present within the detachable support layer, the first drug is coated on the detachable support layer, or a combination thereof. In some embodiments, the first drug is dispersed within a biodegradable polymer. In an embodiment, the nano/microparticles loaded with the second drug are dispersed within a biodegradable polymer. In a particular embodiment, the second drug is the same drug as the first drug, is a different drug than the first drug, or combinations thereof. In some embodiments, the different drug is a small molecule, an antibiotic, an immunomodulatory cytokine, a growth factor or combinations thereof. In an embodiment, the immunomodulatory cytokine is IL-4 and/or IL-10 and the growth factor is transforming growth factor beta (TGF-β). In an embodiment, the small molecule, antibiotic, immunomodulatory cytokine growth factor or combinations thereof is encapsulated in the nano/microparticles, wherein the encapsulated nano/microparticles are dispersed within a biopolymer. In one embodiment, the biopolymer comprises alginate and calcium chloride. In an embodiment, the alginate is a monodisperse heparin-functionalized alginate. In an embodiment, the biopolymer comprises heparin-functionalized mesoporous silica microparticles. In one embodiment, the monodisperse heparin-functionalized alginate is an encapsulated alginate-heparin microparticle. In some embodiments, the encapsulated alginate-heparin microparticle is dispersed in a second biopolymer. In an embodiment, the second biopolymer is alginate, GelMA or a combination thereof. In a particular embodiment, the immunomodulatory cytokine and the growth factor reprogram M1 and M0 macrophages toward M2 macrophages. In some embodiments, the sustained release of the second drug is a release of from 2 weeks to 8 weeks. In one embodiment, the sustained release of the second drug is a release of from 2 weeks to 4 weeks. In some embodiments, the drug-loaded transdermal microneedles have a height ranging from about 300 μm to about 1200 μm. In one embodiment, the drug-loaded transdermal microneedles comprise a base diameter of from about 100 μm to about 400 μm. In an embodiment, the drug-loaded transdermal microneedles have an aspect ratio of height to diameter of about 5 or more. In an embodiment, the plurality of drug-loaded transdermal microneedles comprises from 10 to 90 transdermal microneedles. In an embodiment, the plurality of drug-loaded transdermal microneedles comprises from 100 to 900 transdermal microneedles. In a particular embodiment, the transdermal microneedles loaded with the second drug comprise an antibiotic, antibacterial agent, an anti-inflammatory agent, a growth factor, a cytokine, or any combination thereof. In one embodiment, the second drug in the drug-loaded transdermal microneedles is soluble, is in nanoparticles, is in microparticles, or any combination thereof. In an embodiment, the first drug in the detachable support layer comprises an antibiotic, antibacterial agent, an anti-inflammatory agent, a growth factor, a cytokine or any combination thereof. In some embodiments, the first drug in the detachable support layer is soluble, is in nanoparticles is in microparticles, or any combination thereof. In a particular embodiment, the first drug and/or the second drug is coated on the microneedles, the first drug and/or the second drug is coated on the detachable support, or both, and the first drug and/or the second are independently selected from an antibiotic, antibacterial agent, an anti-inflammatory agent, a growth factor, a cytokine or any combination thereof. In an embodiment, the first drug and/or the second drug is in nanoparticles or in microparticles. In one embodiment, the second drug in the drug-loaded transdermal microneedles, is independently an antibiotic, antibacterial agent, an anti-inflammatory agent, a growth factor, a cytokine or any combination thereof. In one embodiment, the second drug loaded in the transdermal microneedles comprises an antibiotic, an anti-inflammatory agent, a cytokine, a growth factor, or a combination thereof, and the detachable support layer comprises an antibiotic. In an embodiment, the second drug loaded in the transdermal microneedles comprises microparticles comprising an anti-inflammatory agent, a cytokine, a growth factor, or a combination thereof, and the detachable support layer comprises an antibiotic. In some embodiments, the second drug loaded in the transdermal microneedles comprises an anti-inflammatory agent, and the detachable support comprises an antibiotic. In an embodiment, the second drug loaded in the transdermal microneedles comprise a cytokine, and the detachable support comprises an antibiotic. In a particular embodiment, the antibiotic or antibacterial agent is tetracycline, doxycycline, chlorhexidine and/or minocycline. In an embodiment, the periodontal drug delivery system comprises an amount of from about 50 ng up to about 1,500 ng of the antibacterial agent per transdermal microneedle patch. In an embodiment of the periodontal drug delivery system, the anti-inflammatory agent is a steroid and/or a non-steroidal anti-inflammatory drug. In one embodiment, the steroid is dexamethasone hydrocortisone, cortisone, and/or prednisone. In an embodiment, the non-steroidal anti-inflammatory drug is indomethacin, naproxen, ibuprofen, flurbiprofen and/or piroxicam. In some embodiments, the periodontal drug delivery system is administered to the subject, wherein the diseased periodontal tissue comprises periodontal ligament, cementum, gingiva, and/or alveolar bone. In one embodiment, the subject has periodontitis and/or gingivitis.

In one aspect, provided herein is a method for treating periodontitis and/or gingivitis in a subject in need thereof, the method comprising sealably applying to the periodontal tissue a microneedle patch, the microneedle patch comprising: (a) a detachable support layer comprising a surface capable of a sealable application to a diseased periodontal tissue of a subject in need thereof; and (b) a plurality of detachable, drug-loaded transdermal microneedles having bases disposed on the surface of the detachable support layer and projecting perpendicularly therefrom.

In an embodiment, the detachable support layer, the transdermal microneedles, or both, independently comprise a biodegradable polymer. In some embodiments of the periodontal drug delivery system the biodegradable polymer is independently selected from the group consisting of chitosan, alginate, gelatin, hyaluronic acid (HA), polycaprolactone (PCL), poly(lactic-co-glycolic acid) (PLGA), hydrophobically-modified alginate, hydrophobically-modified chitosan, alginate-heparin, chitosan-heparin, methacrylated gelatin (GelMA), and combinations thereof. In an embodiment, the biodegradable polymer dissolves at body temperature, the body temperature ranging from 97° F. (36.1° C.) to 99° F. (37.2° C.). In an embodiment, the detachable support layer is non-membranous and non-adhesive. In certain embodiments, the detachable support layer, the microneedles, or both are porous. In a particular embodiment, the first drug is an antibiotic or an antibacterial agent. In an embodiment, the first drug is present within the detachable support layer, the first drug is coated on the detachable support layer, or a combination thereof. In some embodiments, the first drug is dispersed within a biodegradable polymer. In an embodiment, the nano/microparticles loaded with the second drug are dispersed within a biodegradable polymer. In a particular embodiment, the second drug is the same drug as the first drug, is a different drug than the first drug, or combinations thereof. In some embodiments, the different drug is a small molecule, an antibiotic, an immunomodulatory cytokine, a growth factor or combinations thereof. In an embodiment, the immunomodulatory cytokine is IL-4 and/or IL-10 and the growth factor is transforming growth factor beta (TGF-β). In an embodiment, the small molecule, antibiotic, immunomodulatory cytokine growth factor or combinations thereof is encapsulated in the nano/microparticles, wherein the encapsulated nano/microparticles are dispersed within a biopolymer. In one embodiment, the biopolymer comprises alginate and calcium chloride. In an embodiment, the alginate is a monodisperse heparin-functionalized alginate. In an embodiment, the biopolymer comprises heparin-functionalized mesoporous silica microparticles. In one embodiment, the monodisperse heparin-functionalized alginate is an encapsulated alginate-heparin microparticle. In some embodiments, the encapsulated alginate-heparin microparticle is dispersed in a second biopolymer. In an embodiment, the second biopolymer is alginate, GelMA or a combination thereof. In a particular embodiment, the immunomodulatory cytokine and the growth factor reprogram M1 and M0 macrophages toward M2 macrophages. In some embodiments, the sustained release of the second drug is a release of from 2 weeks to 8 weeks. In one embodiment, the sustained release of the second drug is a release of from 2 weeks to 4 weeks. In some embodiments, the drug-loaded transdermal microneedles have a height ranging from about 300 μm to about 1200 μm. In one embodiment, the drug-loaded transdermal microneedles comprise a base diameter of from about 100 μm to about 400 μm. In an embodiment, the drug-loaded transdermal microneedles have an aspect ratio of height to diameter of about 5 or more. In an embodiment, the plurality of drug-loaded transdermal microneedles comprises from 10 to 90 transdermal microneedles. In an embodiment, the plurality of drug-loaded transdermal microneedles comprises from 100 to 900 transdermal microneedles. In a particular embodiment, the transdermal microneedles loaded with the second drug comprise an antibiotic, antibacterial agent, an anti-inflammatory agent, a growth factor, a cytokine, or any combination thereof. In one embodiment, the second drug in the drug-loaded transdermal microneedles is soluble, is in nanoparticles, is in microparticles, or any combination thereof. In an embodiment, the first drug in the detachable support layer comprises an antibiotic, antibacterial agent, an anti-inflammatory agent, a growth factor, a cytokine or any combination thereof. In some embodiments, the first drug in the detachable support layer is soluble, is in nanoparticles is in microparticles, or any combination thereof. In a particular embodiment, the first drug and/or the second drug is coated on the microneedles, the first drug and/or the second drug is coated on the detachable support, or both, and the first drug and/or the second are independently selected from an antibiotic, antibacterial agent, an anti-inflammatory agent, a growth factor, a cytokine or any combination thereof. In an embodiment, the first drug and/or the second drug is in nanoparticles or in microparticles. In one embodiment, the second drug in the drug-loaded transdermal microneedles, is independently an antibiotic, antibacterial agent, an anti-inflammatory agent, a growth factor, a cytokine or any combination thereof. In one embodiment, the second drug loaded in the transdermal microneedles comprises an antibiotic, an anti-inflammatory agent, a cytokine, a growth factor, or a combination thereof, and the detachable support layer comprises an antibiotic. In an embodiment, the second drug loaded in the transdermal microneedles comprises microparticles comprising an anti-inflammatory agent, a cytokine, a growth factor, or a combination thereof, and the detachable support layer comprises an antibiotic. In some embodiments, the second drug loaded in the transdermal microneedles comprises an anti-inflammatory agent, and the detachable support comprises an antibiotic. In an embodiment, the second drug loaded in the transdermal microneedles comprise a cytokine, and the detachable support comprises an antibiotic. In a particular embodiment, the antibiotic or antibacterial agent is tetracycline, doxycycline, chlorhexidine and/or minocycline. In an embodiment, the periodontal drug delivery system comprises an amount of from about 50 ng up to about 1,500 ng of the antibacterial agent per transdermal microneedle patch. In an embodiment of the periodontal drug delivery system, the anti-inflammatory agent is a steroid and/or a non-steroidal anti-inflammatory drug. In one embodiment, the steroid is dexamethasone hydrocortisone, cortisone, and/or prednisone. In an embodiment, the non-steroidal anti-inflammatory drug is indomethacin, naproxen, ibuprofen, flurbiprofen and/or piroxicam. In some embodiments, the periodontal drug delivery system is administered to the subject, wherein the diseased periodontal tissue comprises periodontal ligament, cementum, gingiva, and/or alveolar bone. In one embodiment, the subject has periodontitis and/or gingivitis.

The foregoing description is merely non-limiting as to the variations in the design and construction of the micropatch for the intended uses described herein.

As noted herein, one or more drugs may be provided in one or more locations in the microneedle patch. A drug may be provided in the microneedles, on the microneedles, in the detachable support layer or on the detachable support layer, in any of various forms, including but not limited to soluble, coated, in or on a microparticle, or in or on a nanoparticle. Moreover, any combination of any drugs may be ant any one or more sites in or on the micropatch. The term “independently” is used to mean that the selection of drug at two or more sites may be the same or different, or any combination, in any form. In other embodiments, the microneedles, the detachable support layer, or both, may be porous.

As described herein, the microneedle patch (also called a micro-patch) may be used to delivery one or more drugs to the periodontal area, for uses such as but not limited to regenerating periodontal tissue, for reducing local inflammation in periodontal tissue, for promoting regeneration of periodontal bone loss, or for treating periodontitis or gingivitis.

Each of the components of the micro-patch are described below. The descriptions of each component are not intended to be limiting, and variations therein are fully embraced within the invention.

Micro-Patch Fabrication

Fabrication of microneedle patches are described in the art, such as in Li et al., Nature Biomedical Engineering 3:220-229 (2019); Chen et al., Journal of Materials Chemistry B, Issue 3, (2017); and Xue et al., RSC Advances, Issue 92 (2015), incorporated herein by reference.

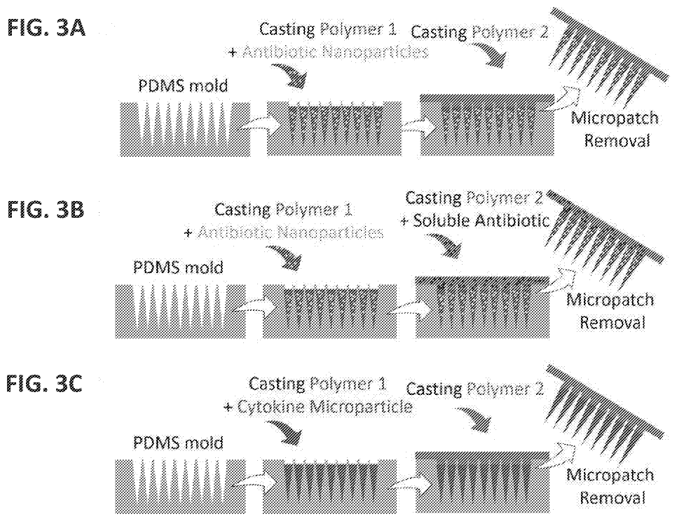

General description. A schematic description of several variations in the micro-patch design are shown in FIG. 3. FIG. 3A show a drug (soluble or encapsulated in nanoparticles; antibiotic nanoparticles are depicted) is mixed with polymer 1 and then cast over the mold (made for example of polydimethylsiloxane [PDMS]). The drug is encapsulated within the microneedle polymer. Polymer 2 acts as a detachable support layer and will be cast over the mold. Upon removal from the mold, the detachable support presents the array of microneedles projecting from one side.

In FIG. 3B, the microneedles contain drug as described in FIG. 3A, but in addition, a soluble drug is mixed with polymer 2 and then cast over the mold that already has formed microneedles. Therefore, in this case, the support layer will be drug-loaded. In this embodiment, the drug in the support layer is soluble, but in other embodiments, the drug may be nanoparticles or microparticles included in the support layer. The support layer (based on, in some embodiments, gelatin or PVP) will dissolve quickly in the body, the detachable support drug (for example, an antibiotic) will be released in a burst.

FIG. 3C shows a micro-patch wherein a cytokine is present in the microneedles. In one embodiment, a protein therapeutics (e.g., a cytokine) is encapsulated into microparticles and then mixed with polymer 1 during the casting over the mold. Therefore, after the formation of microneedles, the drugs are encapsulated. The polymer 2 as a support layer will cast over the mold. In this example, the detachable support has no drug.

In another embodiment, the microneedles may be coated with a drug that, upon implantation of the micro-patch, releases into the tissue quickly, providing a burst. In another embodiment, the detachable support may also be coated with a drug, for release upon placement.

Included in the variations of the aforementioned formats of the micro-patch include combinations of the various mentioned methods. As noted herein, the drugs in the microneedles, coated on the microneedles, in the detachable support, and coated thereon, may be independently selected.

The size of the micropatch can be seen in the schematic in FIG. 1, but it is not so limited and may be of a size, and contain the number of microneedles, to be therapeutically effective.

Selection of polymer for microneedles. In one embodiment, alginate, chitosan, and methacrylated gelatin polymers (due to their biocompatibility as well as ease of use and modify) can be used to fabricate the microneedles of the micro-patch described herein. Other polymers include gelatin, hyaluronic acid (HA), polycaprolactone (PCL), poly(lactic-co-glycolic acid) (PLGA), chitosan, chitosan-heparin, and methacrylated gelatin are non-limiting examples of polymers that can comprise the microneedles of the micro-patch.

The aforementioned polymers and others have been used in the art to fabricate microneedle patches for a wide range of small molecule or protein transdermal delivery. Alginate generally has slow biodegradation (several months) but amylase present in human saliva can facilitate hydrolysis of the polysaccharide, including alginate, in the mouth environment. Other polymers may be selected based on the desired mechanical properties, release properties, degradation properties, among other factors. As shown in the examples herein, solid microneedle patches were produced, optimized and characterized based on these polymers. For example, different concentrations of alginate (0.5-5 wt. %) mixed with the drug-loaded nanoparticles (NPs) described herein, and cast over polydimethylsiloxane (PDMS) molds with designated dimension (see below).

In one embodiment, microneedles are dried at room temperature for 20 hours and then removed for the molds. Crosslinking of alginate-based microneedles using various concentrations (e.g., 5-100 mM) of calcium chloride following by second cycle of drying in room temperature performed after removing the needles from the mold to make microneedles with various stiffnesses and mechanical properties. As will be described below, the microneedles may be attached to a support for ease in handling, tissue placement, and also for additional drug delivery properties. This support is referred to herein as a detachable support.

To make porous microneedles, the solid (non-porous) microneedles should be formed containing a porogen. In one embodiment, after forming microneedles, the porogen can be dissolved to create pores. Another way to create porous microneedles will be using the freeze-drying (lyophilization) process. After forming the microneedles, they will be removed from the mold, pre-wet, frozen at −80 C and then put in a primary chamber of a freeze-dryer machine to create pores. However, the porous microneedles may have lower mechanical properties that limit its tissue penetration. These are merely non-limiting methods for creating porosities in the microneedles. In one embodiment, porous microneedles degrade more rapidly than non-porous needles. In another embodiment, porous microneedles integrate with tissues better than non-porous microneedles.

Selection of other components present in microneedles. Drugs such as anti-inflammatory compounds, cytokines or growth factors, by way of non-limiting examples may be incorporated into the microneedles. In one embodiment an antibiotic is present. In one embodiment one drug is present in the microneedles; in other embodiments, more than one drug is present. The drug may be incorporated into the polymer during casting or introduced into the polymer afterwards. In one embodiment, microparticles as described below containing anti-inflammatory compounds, cytokines or growth factors (referred to herein as drugs) may be included in the microneedles by mixing with the polymer before casting the microneedles. Microparticles are provided to delay the delivery of the drug from the microneedles into the gingival tissue. For more rapid release, the drug may be incorporated directly into the polymer without biding to or encapsulation into particles (i.e., soluble in the polymer).

In another embodiment, a drug may be coated onto the microneedles after casting, so as to provide another means to deliver a drug to the site. In one embodiment, the microneedle-coated drug provides an immediate release into the tissue.

Microneedle physical characteristics. The microneedle size and shapes may be selected based upon the desired components and their release properties. In one embodiment, having sharper microneedles with aspect ratio (height to base diameter) of more than 5 facilitates easier penetration. In one embodiment, the aspect ratio is more than about 3.

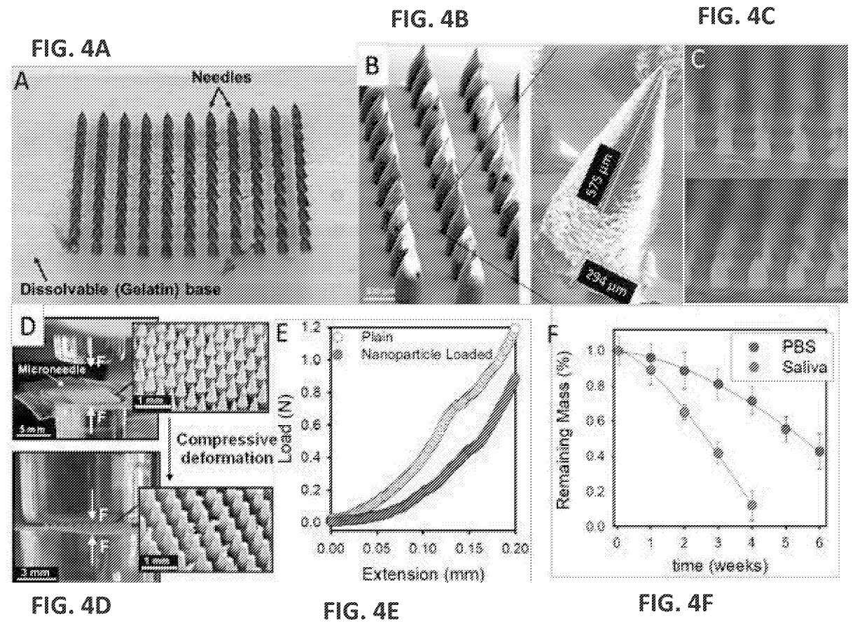

In one non-limiting embodiment, and as analyzed using scanning electron microscopy (SEM) (FIG. 4B) as well as fluorescent microscopy (FIG. 4C) for comparison between different formulations. In one embodiment, microneedle dimension (for example, height: 400-1200 μm; base diameter: 150-500 μm) and composition are optimized to tune the mechanical properties (for example, FIG. 4E) as well as degradation profile. In one embodiment, the microneedle dimensions and composition, as well as the number of microneedles comprising a micro-patch, are provided to optimize the delivery of the drug into tissue. In one example, FIG. 4E shows a slight decrease of mechanical property of microneedles loaded with PLGA NPs due to disturbing of the alginate crosslinking networks.

The size of the array of microneedles may vary depending on the number of microneedles and the number of rows in a micro-patch. For example, a micro-patch comprising 121 microneedles arranges in a 11 by 11 needle array would measure 1 square cm; with the detachable support overlapping the array dimensions, a size of 2.25 square cm.

Adjustment and assessment of microneedle mechanical properties. The stiffness of the microneedles may be tuned to the level sufficient or required for tissue penetration by, in one embodiment, adjusting the concentration of alginate and Ca²⁺. In other embodiments, the combination of polymers may be used to provide the appropriate mechanical properties. In one embodiment, the mechanical (compression) properties of the microneedles can be measured by an Instron (Model 5542).

Casting the detachable support. After drying the alginate microneedles inside the molds (and, in one embodiment, before further treatment of the microneedles such as cross-linking), in one embodiment, a detachable support is cast over the microneedles to form the micro-patch that can be readily positioned at the desired site, the microneedle array penetrating into the tissue and the detachable support affixed to the surface of the tissue. In one embodiment, the degradation of the detachable support results in its detachment from the tissue and microneedles, leaving the microneedles in place. In one embodiment, the detachable support provides the means by which to place the microneedles in the desired location. In one embodiment, the detachable support further contains a drug that is released from the detachable support into the tissue before the detachable support degrades or detaches from the tissue. In one embodiment, the detachable support comprises a gelatin layer containing water-soluble tetracycline-HCl which is formed to cover the molds as a supporting film (air dried) to make the microneedle easy to handle and also to provide burst release of antibiotics inside the gingival pocket upon dissolution of this support layer. This supporting film is also referred to as a detachable support. In one embodiment the drug in the detachable support may be bound or encapsulated in order to delay release. The dried gelatin film will provide proper mechanical strength as support a material. In one embodiment, the gelatin layer dissolves at body temperature in <15 minutes and releases the tetracycline. The foregoing examples are merely exemplary and non-limiting of the methods for fabricating the micro-patch of the invention, wherein the microneedles and detachable support may be fabricated by other methods to achieve the same desired properties.

Coating of microneedles. In another embodiment, the microneedles are coated with a drug. In one embodiment, after forming drug-free or drug-loaded microneedles, the surface of microneedles is coated with a Drug. This drug will be physically adsorbed on the surface of needles and will be released shortly (within minutes to several of hours) or upon dissolution/disintegration of microneedles.

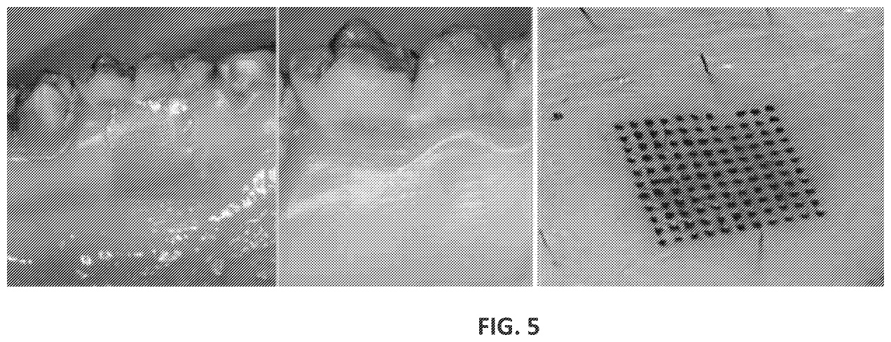

Adjustment and assessment of micropatch adhesion properties. The adhesion of the patches to the extracted pig or rat's gingival tissues and penetration of the microneedles into the tissues can be characterized, in one embodiment, using an animal model, and after placement of the micro-patch and collection of gingival tissue, using cryosectioning, staining, and analyzing of the tissue. As shown in the examples, results on extracted pig gums showing successful penetration of placement of microneedles after dissolution of their gelatin-based support layer (FIG. 5).

Adjustment and assessment of microneedle degradation properties. Degradation of microneedles at physiologically relevant conditions, 37° C., in either (artificial) saliva or PBS also can be evaluated to optimize the polymer formulation. As tissue/mouth pH fluctuates significantly during normal conditions or in periodontal disease, effects of pH (pH: 3.0 to 9.5) on release and degradation of microneedles may be assessed. A degradation study may be performed to measure the degradation rate of the material in PBS, artificial saliva and also in pooled human saliva (Innovative Research, Inc. Novi, Mich.) at 37° C., over 8 weeks on a shaker incubator. As noted in the examples, there is a significant difference in degradation rate of alginate-based biomaterials in PBS vs. human saliva (FIG. 4F).

Assessment of compatibility of micro-patch with gum tissues. The in vitro biocompatibility of the selected microneedle patch candidates can be tested in vitro using rat gingival mesenchymal stem cells (rGMSCs). rGMSCs are isolated and cultured using the same protocol as described for human-derived GMSCs. Based on in vitro experiments, optimized formulations can be prepared that are non-cytotoxic (viability>90%) and support cellular activities (cell proliferation). The material formulations that meet these criteria can then be used for in vivo biocompatibility testing.

Incorporated Drug Nanoparticles (NPs) to Prolong Delivery.

Polymeric NPs may be synthesized using, in various embodiments, top-down or bottom-up processing. In one embodiment, top-down processing involves making millimeter/micrometer size particles and then breaking/grinding them to obtain nanoparticles. In one embodiment, bottom-up processing involves assembly at the molecular level to form nanoscale particles. In one embodiment, bottom-up processing involves the self-assembly of particles via intermolecular forces, and offers a great opportunity to tailor the particle structure and properties. In one example, formation of chitosan nanoparticles is described using microfluidics in a bottom-up process, as described in Majedi et al., 2013, Advanced Functional Materials 24(4):432-444.

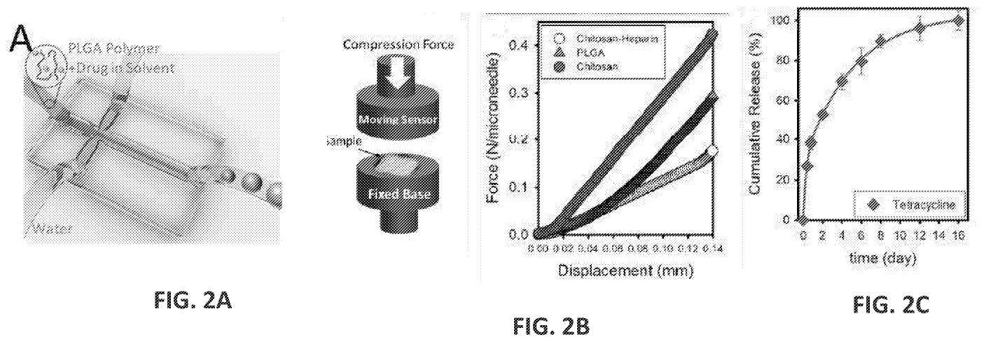

In one embodiment, NPs are synthesized by top-down processing. In one embodiment, small and monodisperse NPs are created with microfluidic techniques. This well-controlled mixing regime can be precisely adjusted on microfluidic platforms (primarily through flow ratio and velocity), which allows the generation of monodisperse NPs with tunable structural features, improved drug encapsulation efficiency and adjustable release pattern. Monodisperse NPs can be created with microfluidic technique (FIG. 2A). This allows for controlling size (by way of non-limiting example, 60-200 nm), zeta potential (by way of non-limiting example, 1-14 mV), drug loading efficiency (by way of non-limiting example, >95%), and sustained release profile. In one embodiment, the release of a model antibiotic (tetracycline) at different initial loading was studied (FIG. 2C). NPs were imaged using transmission/scanning electron and atomic force microscopies. The mechanical (compression) properties of the microneedles was measured by an Instron (Model 5542) as shown in FIG. 2B.

In one embodiment, particles based on poly(lactic-co-glycolic) acid (PLGA) (due to its controlled biodegradation and compact structure) may be developed, with a wide range of sizes with the use of microfluidic platform, and used thereby to determine how size and monodispersity may affect release and effectiveness of a model antibiotic (tetracycline) drug over time. In addition, the manipulation of molecular weight of PLGA and the ratio between lactic acid and glycolic acid in PLGA copolymer offers further control of the release profile. Since the regeneration of periodontal tissues will take 2-6 weeks, in one embodiment, a target>2 weeks of drug release is desirable. This targeted release period can be easily tailored in the drug delivery system as needed. For example, NPs were loaded with three different levels of tetracycline (5, 15, 20 wt_{drug}/wt_{PLGA}%) as representative low, medium, and high dose of antibiotics. The stability of the NPs in the phosphate buffered saline (PBS) and salivary environment were monitored through dynamic light scattering (DLS) and zeta potential measurements (Zetasizer Nano, Malvern). Natural saliva has a complex and variable composition, and the use of an artificial saliva with known composition should therefore facilitate the understanding of the influence of the salivary constituents on the stability and release of antibiotics from designed NPs. Artificial saliva was prepared as described in the examples below. The release kinetics and in vitro characteristics of these NPs studied under gentle shaking at 37° C. in PBS and in artificial saliva. To increase the production rate of PLGA NPs, microvortex platform or parallel flow focusing system were also developed to reach to a productivity of ˜1 g/hour/chip while providing the expected reproducibility and homogeneity of NPs.

Evaluation of loading of micro-patch components with drug. In order to provide antibacterial activity in the micro-patches, each patch, including the detachable support layer (for example made of gelatin) and microneedles (made of gelatin-methacrylate or alginate) can be loaded with various dosages of tetracycline to provide, for example, three different levels: low (50 μg/patch), medium (300 μg/patch), and high-dose (1.5 mg/patch) delivery. Due to the higher drug loading capacity of the gelatin base, in one embodiment, ≈80% of the drug will be loaded there to provide a strong early-stage antibacterial effect. Studies confirm that we can load up to 1.5 mg tetracycline per patch which will meet the required dose for human use. For example, state-of-the-art Arestin® (Valeant Pharma Inc.) microspheres contain 1 mg of minocycline per therapy. In one embodiment, the range of drug amount may be be 50 ug/patch to 1.5 mg/patch. In one embodiment, the dosage per microneedle range would be 50-500 ng per microneedle array, e.g., and 11×11 array.

Selection of Drugs. As noted herein, the microneedles, the coating of the microneedles, the detachable support, and the coating of the detachable support may independently comprise one or more drugs to achieve the therapeutic properties of the micro-patch. Each site of the micro-patch may have a different drug or combination of drugs, or the same drug or combination of drugs, or combinations thereof; the same drug may be in a different delivery system (e.g., soluble, microparticle, or nanoparticle) in different sites in the micro-patch. As noted elsewhere, some drugs may be provided as a coating or soluble in the detachable support of microneedles to provide a burst or short-term release; other drugs may be provided in a controlled release form such as in a microparticle or nanoparticle, to delay release over a longer duration.

Non-limiting examples of drugs that can be provided in a micro-patch are described below.

Antibiotics. Antibiotics such as tetracycline, doxycycline, chlorhexidine and/or minocycline my be provided in any one or more sites of the micro-patch. It may be soluble, or provided in a controlled delivery system such as a microparticle or nanoparticle.

Anti-inflammatories. In some embodiments, the anti-inflammatory is a steroid or a non-steroidal anti-inflammatory. Non-limiting examples of steroids include dexamethasone hydrocortisone, cortisone, and/or prednisone. Non-limiting examples of non-steroidal anti-inflammatories include indomethacin, naproxen, ibuprofen, flurbiprofen and/or piroxicam.

Cytokines. In some embodiments, a cytokine such as interleukin 4 (IL-4) or interleukin 10 (IL-10) may be provided in the micro-patch. In one embodiment, the cytokine is in a microparticle. In one embodiment the cytokine is provided in a silica microparticle. In one embodiment, the cytokine is provided in a nanoparticle. Such delivery systems as described below.

Growth factors. In some embodiments, growth factors other than cytokines include transforming growth factor beta (TGFβ), insulin-like growth factor 1 (IGF-1), and/or bone morphogenetic protein-2 (BMP-2). In one embodiment, the growth factor is provided in a microparticle. In one embodiment the cytokine is in a silica microparticle. In one embodiment, the growth factor is provided in a nanoparticle. Such delivery systems as described below. In order to facilitate the recovery of lost bone tissue, incorporation of recombinant bone morphogenetic protein-2 (rBMP-2) is employed.

For some therapeutic proteins such as cytokines and growth factors, activity maybe decreased during the fabrication process. To protect proteins from such effects, in one embodiment, cytokines and other proteins can be loaded along with trehalose as a protein chaperon. Its critical property that helps stabilize proteins as a biological preservative is exploited. In other embodiments, support polymers like polyvinylpyrrolidone instead of trehalose may be used.

As described above, the microneedles of the micro-patch comprise a drug, and the other components of the micro-patch may or may not comprise a drug: the microneedle coating, the detachable support and the coating of the detachable support. Thus, in some embodiments, the microneedles comprise a drug selected from an antibiotic, antibacterial agent, an anti-inflammatory agent, a growth factor, a cytokine or any combination thereof, and the other components do not comprise any drug. In some embodiments, these drugs may be soluble in the microneedles, or may be present in or on microparticles or nanoparticles.

In one embodiment, the microneedles and the detachable support comprise, independently, an antibiotic, antibacterial agent, an anti-inflammatory agent, a growth factor, a cytokine or any combination thereof, and the microneedles and the surface of the detachable support do not comprise a drug. In some embodiments, these drugs may be soluble in the microneedles and/or the detachable support, or may be present in or on microparticles or nanoparticles in the microneedles or in the detachable support.

In another embodiment, the microneedles, surface of the microneedles and the detachable support comprise, independently, an antibiotic, antibacterial agent, an anti-inflammatory agent, a growth factor, a cytokine or any combination thereof, and the surface of the detachable support does not comprise a drug. In some embodiments, these drugs may be soluble in the microneedles and/or the detachable support, or may be present in or on microparticles or nanoparticles in or on the microneedles or in the detachable support.

In one embodiment, the microneedles, the microneedles, detachable support and surface of the detachable support comprise, independently, an antibiotic, antibacterial agent, an anti-inflammatory agent, a growth factor, a cytokine or any combination thereof, and the microneedle surfaces do not comprise a drug. In some embodiments, these drugs may be soluble in the microneedles and/or the detachable support, or may be present in or on microparticles or nanoparticles in the microneedles or in or on the detachable support.

In one embodiment, the microneedles comprise a cytokine and the detachable support comprises an antibiotic. In one embodiment, the cytokine is in microparticles, and the antibiotic is soluble. In one embodiment the cytokine is IL-4, IL-10, TGFβ, IGF-1 or BMP-2, or any combination thereof; the antibiotic is tetracycline, minocycline, chlorhexidine or doxycycline. In one embodiment, the microneedles comprise a cytokine microparticle or nanoparticle, and an antibiotic nanoparticle, and the detachable support comprises a soluble antibiotic.

In one embodiment, the microneedles comprise a cytokine in microparticles, and the detachable support comprises a soluble antibiotic. In one embodiment, the microneedles comprise a cytokine in microparticles. In one embodiment, the microneedles comprise a soluble cytokine, and the detachable support comprises a soluble antibiotic. In one embodiment, the microneedles comprise a soluble cytokine. In one embodiment, the microneedles comprise an antibiotic in nanoparticles, and the detachable support comprises a soluble antibiotic. In one embodiment, the microneedles comprise an antibiotic in nanoparticles. In one embodiment, the microneedles comprise a soluble antibiotic, and the detachable support comprises a soluble antibiotic. In one embodiment, the microneedles comprise a soluble antibiotic. In one embodiment, the microneedles comprise a cytokine in microparticles and an antibiotic in nanoparticles, and the detachable support comprises a soluble antibiotic. In one embodiment, the microneedles comprise a cytokine in microparticles and an antibiotic in nanoparticles. In one embodiment, the microneedles comprise a cytokine in microparticles, the detachable support comprises a soluble antibiotic, and the microneedles are coated with a soluble antibiotic. In one embodiment, the microneedles comprise a cytokine in microparticles, and the microneedles are coated with a soluble antibiotic. In one embodiment, the microneedles comprise a soluble cytokine, the detachable support comprises a soluble antibiotic, and the microneedles are coated with a soluble antibiotic. In one embodiment, the microneedles comprise a soluble cytokine, and the microneedles are coated with a soluble antibiotic. In one embodiment, the microneedles comprise an antibiotic in nanoparticles, the detachable support comprises a soluble antibiotic, and the microneedles are coated with a soluble antibiotic. In one embodiment, the microneedles comprise an antibiotic in nanoparticles, and the microneedles are coated with a soluble antibiotic. In one embodiment, the microneedles comprise a soluble antibiotic, the detachable support comprises a soluble antibiotic, and the microneedles are coated with a soluble antibiotic. In one embodiment, the microneedles comprise a soluble antibiotic, and the microneedles are coated with a soluble antibiotic. In one embodiment, the microneedles comprise a cytokine in microparticles and an antibiotic in nanoparticles, the detachable support comprises a soluble antibiotic, and the microneedles are coated with a soluble antibiotic. In one embodiment, the microneedles comprise a cytokine in microparticles and an antibiotic in nanoparticles, and the microneedles are coated with a soluble antibiotic.

In one embodiment, the microneedles comprise IL-4 in microparticles, and the detachable support comprises soluble tetracycline HCl. In one embodiment, the microneedles comprise IL-4 in microparticles and tetracycline HCl in nanoparticles, and the detachable support comprises soluble tetracycline HCl.

Microparticles for Anti-Inflammatory, Cytokine or Growth Factor Delivery

In one embodiment, the cytokine or growth factor is provided as a loaded mesoporous silica microparticles. In one embodiment, the silica has enhanced affinity for the cytokine or growth factor. In another embodiment, heparin can significantly increase the affinity of positively charged proteins having an isoelectric point (pI)>7.5. In one embodiment, heparin-functionalized mesoporous microparticles (10 μm in diameter) are synthesized and optimized to encapsulate target cytokines (IL-4; pI: 9.17) and growth factors (TGF-β; pI: 8.5). These particles are then loaded in microneedles patch as described herein.

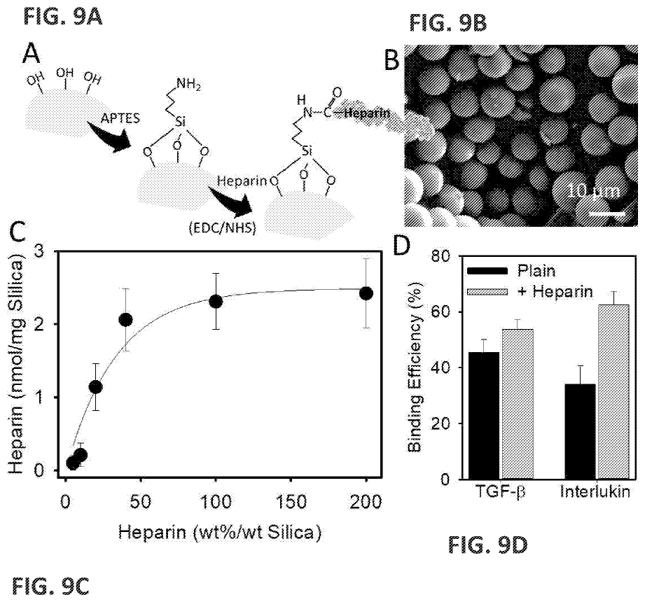

In one embodiment, monodisperse mesoporous silica microparticles (5 to 50 μm) are produced via a microfluidic jet spray-drying route, using cetyl trimethylammonium bromide (CTAB) and/or Pluronic F127 as templating agents, and tetraethylorthosilicate (TEOS) for silica as reported before. In one embodiment, heparin-based conjugates (e.g., silica-heparin) developed at several conjugation densities (for example, see FIG. 9C). Carbodiimide chemistry (NHS/EDC) may be utilized to modify silica conjugates with heparin after treating the silica with (3-Aminopropyl)triethoxysilane (APTES) to provide primary amine groups (FIG. 9A,B). The presence of heparin provides enhanced efficiency and stability of cytokine binding (as exemplified in FIG. 9D) that enables precise spatiotemporal control over the release profile of target proteins (IL-4 and/or TGF-β). As described in the examples, IL-4 and TGF-β proteins (5-100 nM) were dissolved in 500 μl PBS and then loaded into mesoporous silica microparticles by overnight incubation at 4° C. Particles were then washed with PBS and stored at 4° C. prior use. Absorption (binding) of cytokines was studied after dissolving the microparticles in hydrofluoric acid using ELISA kits (R&D Systems Minneapolis, Minn.) for each protein individually.

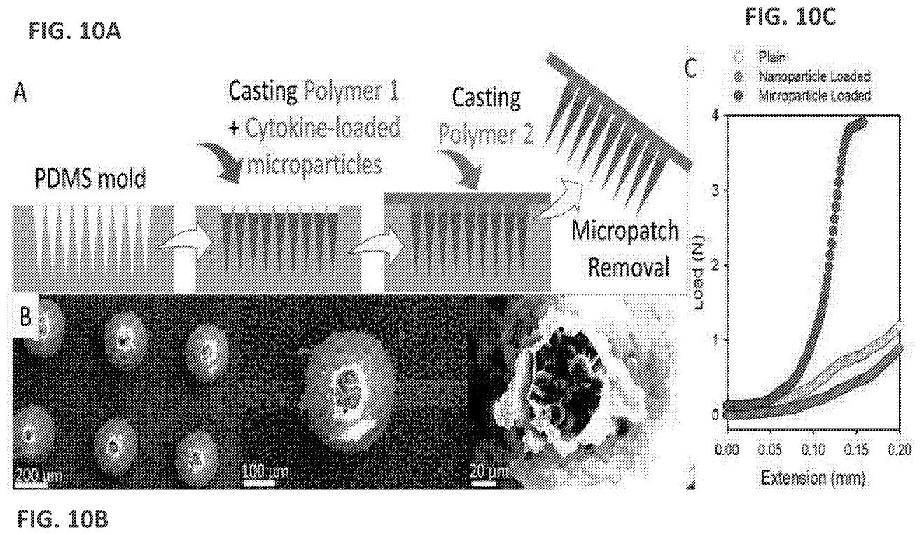

Evaluation of cytokine release. For high quality periodontal treatment, the periodontal patch should in one embodiment act as an immunomodulator by delivering small molecules drugs and/or bioactive molecules to control infection, modulate inflammation and induce the recovery of alveolar bone. Following the guidance in FIG. 3A to fabricate microneedles. IL-4- or TGF-β-loaded silica-heparin microparticles were mixed with polymer (e.g., alginate) solution and cast into molds to generate the microneedle patches. The kinetics of cytokine release from microneedles with different composition and geometry may be studied using ELISA for each protein individually and in combination to assess the possible protein-protein interaction. The mechanical properties of the generated microneedle patch containing cytokines at different loading densities were evaluated. Results confirm the successful encapsulation of cytokine-loaded silica particles into alginate microneedles (FIG. 9B). The presence of these particles also increased the mechanical properties of resulted microneedles by 7 folds (FIG. 10C). The chemical formulation of microneedles was further modified for co-encapsulation of both small molecule antibiotics (encapsulated in PLGA NPs) as well as cytokines (encapsulated in heparin-silica microparticles). To characterize the release profile of loaded proteins, microparticles alone or encapsulated (in microneedles) with varying level of protein loading incubated in 500 μL of artificial saliva in 48-well plates on a rotational shaker at 37° C. for up to four weeks. At selected time intervals (every 12 hour), the medium was collected and analyzed for released proteins using ELISA Kits. At the end of release study, the remaining proteins extracted from the particles/needles by dissolving them and the percentage of cumulative released proteins measured according to methods explained herein. The released percentage measured based on the ratio of the amount released at each time point to the amount that was initially loaded. Based on these studies, the formulations that provided controlled release over 2-4 weeks were selected. The prolonged release of IL-4 and TGF-β from designed platforms was demonstrated (FIG. 9B).

Sterilization of Micro-Patches

For clinical use, in one embodiment, micro-patches are provided as a sterile product. Depending on the sensitivity of the drugs and polymers used in fabrication, a suitable method of sterilization may be selected. In one embodiment, x radiation is used. For example, x-ray irradiation using Gulmay Medical RS320 x-ray unit may be used. As described in the examples, following the recommended protocol in ISO 11137-2:2013, a sterilization dose of 25 kGy (2.5 Mrads) was used. It has been reported that this dose does not alter the properties of tetracycline. Physical properties, including change in morphology and mechanical stiffness of microneedles patch as well as change in antibacterial may be tested after the sterilization process. As shown in the examples, a non-significant change in mechanical properties of alginate patches was observed after receiving three cycles of 25 kGy sterilization dose (FIG. 8). The anti-bacterial property of sterilized patch may also be verified.

In Vitro Evaluation of Delivery Properties of Micro-Patch.

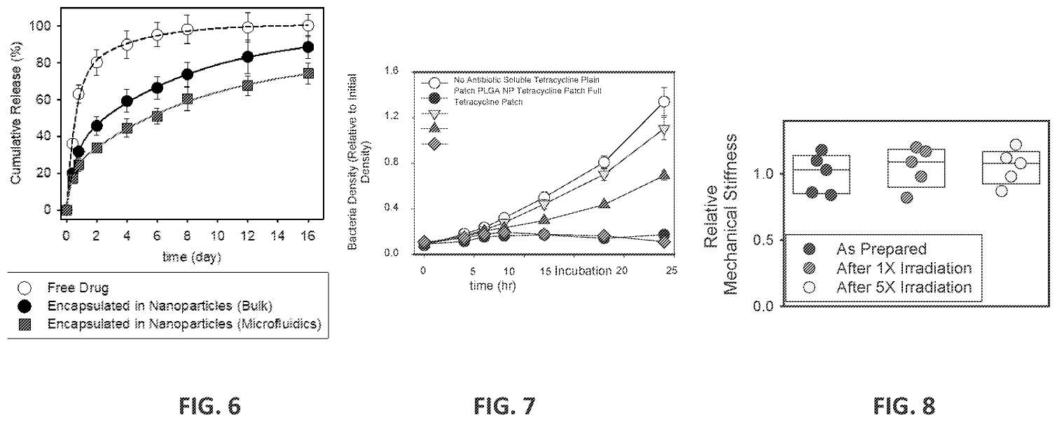

Antibacterial agent delivery. In order to evaluate antibacterial activity by the micro-patches, each patch (including gelatin support layer and alginate needles) is loaded with various dosages to provide three different levels of tetracycline for low (50 μg/patch), medium (300 μg/patch), and high-dose (1.5 mg/patch) delivery. Due to higher drug loading capacity of gelatin base, ≈80% of drug was loaded to provide strong early stage antibacterial effect. Results confirm that up to 1.5 mg tetracycline can be loaded per patch which meets the required dose for human use. For example, state-of-the-art Arestin® (Valeant Pharma Inc.) microspheres contain 1 mg of minocycline per therapy. Here, multistage release of antibiotic was achieved by a burst release during the dissolution of gelatin supporting film in less than 15 minutes followed by prolonged release over 4 weeks. Our observations revealed the advantage of using microfluidic NPs over traditionally fabricated (bulk) NPs or free drug encapsulation in alginate-based needles (FIG. 6). The kinetics of drug release from microneedles with different composition and geometry were studied in vitro using HPLC via a modified Franz cell method.

Antibacterial properties: P.g. and A.a. may be used for the analysis of antibacterial properties. A direct quantitative evaluation of the antimicrobial activity of designed patches is performed. Patches are placed in 2 ml brain heart infusion (BHI) broth inoculated with P.g. or A.a. at an initial concentration optical density at 600 nm (OD600) of 0.3. To control the accuracy of bacterial density, bacteria serially diluted in PBS to 10³, 104, 10⁵, and 10⁶ colony forming units (CFU/ml) and plated overnight (200 μl/plate). Suspensions then incubated at 37° C. for 24, 72, and 120 hours and the change in OD600 was monitored. Assessment of direct inhibition of bacterial growth also performed by counting CFU. LB-agar plates prepared by spreading a 0.5 McFarland (optical density at 600 nm of approximately 0.08-0.1) suspension of bacteria (P.g. or A.a.) in broth using a sterile swab. Bactericidal activity visually assessed to determine the zone of inhibition 1 and 5 days after contact of the patches with the inoculated bacterium. At least four replicates were tested for each experimental group, and then the diameter of the inhibition zone was analyzed. FIG. 7 shows the results of an evaluation against P.g.

In vivo evaluation of delivery properties of micro-patch. In vivo biocompatibility and degradation of the engineered patches can be assessed in rat periodontal tissue. The degradation rate and host response for implanted microneedle patches can be tested first in the absence of drug. The rat periodontal implantation model in presence of microneedle (without drug) is used. For implantation, anesthesia is induced and maintained with isoflurane. Freshly prepared micro-patch samples are implanted by firmly attaching to periodontal tissues. After 7, 14 and 28 days, the constructs are explanted (if not fully degraded) and analyzed for biocompatibility and biodegradation. For the in vivo biocompatibility test, three groups of materials are tested: 1) Sham, and 2) Alginate (two formulations for each selected from the in vitro tests). Each implant type is followed up for four different time periods: 3, 7, 14 and 28 days. The number of animals per group is n=8 based on power analysis. Formulations which showed the lowest inflammatory response and degraded between 21 to 28 days are selected for further studies. As shown in the examples below, data shows subcutaneously implanted alginate constructs did not caused any immune response as there was no sign of lymphocyte infiltration (CD3) and macrophage (CD68) at day 7.

In vitro assessment of immunomodulatory properties of patches. In one embodiment, Costar transwell inserts (pore size 1 μm) may be used for assessing the effectiveness of the released factor(s) on the polarization of macrophages and differentiation of T cells. Patches containing cytokines (4 experimental groups: no drug, IL-4 alone, TGF-β alone and a combination of IL-4/TGF-β) are placed in the upper chamber. The bottom chamber contains artificial saliva modified media and 10⁵ of either primary monocytes or naïve T cells or both (5×10⁴). The isolation of primary monocytes is performed. Naïve T cells are isolated using EasySep Rat T Cell Isolation Kit (STEMCELL Technologies) according to manufacturer's protocol. The change in macrophage phenotypes are tested at different time points (day 1-5) using flow cytometry by checking the surface markers (M1): major histocompatibility complex class II (MHC II), CCR7, CD80 and CD86; M2: CD163 (M2c) and CD206 (M2a)). The change in macrophage function is characterized by phagocytosis capability, inducible nitric oxide synthase (iNOS) production and inflammatory secretome (M1: IL-1-β, tumor necrosis factor (TNF), IL-6, and IL-12; M2: IL-4, IL-10, and TGF-β) analysis. Quantitative PCR (qPCR) is used to determine the gene expression of these inflammatory cytokines and cellular markers (as exemplified in FIG. 9D). Activation and differentiation of T cells are tested by considering expression of CD25 surface activation marker as well as intracellular expression of the transcription factor Foxp3. In addition, soluble cytokines are used as controls to compare the bioactivity of encapsulated proteins. Studies in the examples below show successful reprograming of M1 (and M0) macrophages toward M2 upon treatment with IL-4 (FIG. 9D). The ability of controlling differentiation of naïve T-cells into induced Treg may also be tested by providing sustained release TGF-β from alginate-based particles by rigorously culturing naïve CD4+ T-cells with anti-CD3 and anti-CD28 for 4 days in the wells containing the microparticles loaded with TGF-β at different concentrations and comparing them to the soluble (no particles) counterparts at identical concentrations. The expression of Foxp3 is measured by flow cytometry. As seen in the examples, at all concentrations of TGF-β as provided by the particles, Foxp3 expression was much higher. In contrast, at very high soluble concentrations of TGF-β, Foxp3 expression plateaued and was even slightly suppressed. Furthermore, the MFI of Foxp3 directly relates to the suppressive capacity of the regulatory T-cells. The MFI of Foxp3 was highest when using particles to deliver TGF-β. These results show that induced regulatory T-cells can be potently generated using microparticles that secrete TGF-β. The dose of IL-4 and TGF-β was varied to reach an optimal level to maximize the conversion into M2 type macrophages.

In vitro assessment of the effects on gingival cells. To study how alteration of macrophage polarization and regulatory T cell formation may affect host cells in periodontal tissue, GMSCs are cultured in the absence or presence of treated macrophages/T cells in a Transwell co-culture system with macrophages/T cells in the top well for various lengths of time. Before co-culture experiment, these macrophages/T cells are treated with IL-4/TGF-β-containing (or no-drug control) microneedles for a time period that shows effective conversion into M2 macrophages. After 2 days co-culture of macrophages, T cells, and GMSCs, the survival, proliferation, and matrix production (collagen I) of GMSCs in vitro is examined Cellular viability and metabolic activity are measured over the course of experiment using, in one embodiment, Live/Dead Assay Kit and Presto Blue assay, respectively. Collagen I production is also assayed by quantitative polymerase chain reaction (qPCR). As shown in the examples, the optimized delivery platform can enhance the functions of host GMSCs for tissue regeneration.

All scientific publications cited herein are hereby incorporated by reference in their entireties.

The following examples are presented in order to more fully illustrate certain embodiments of the invention. The examples should in no way be construed, however, as limiting the broad scope of the invention.

### EXAMPLES

### Example 1

### Formulation of Patch: Design and Optimize Antibiotic-Loaded Monodisperse NPs to Prolong the Delivery

Monodisperse NPs were created with microfluidic technique (FIG. 2A). This well-controlled mixing regime can be precisely adjusted on microfluidic platforms (primarily through flow ratio and velocity), which allows the generation of monodisperse NPs with tunable structural features, improved drug encapsulation efficiency and adjustable release pattern. The possibility of controlling size (60-200 nm), zeta potential (1-14 mV), drug loading efficiency (>95%), and sustained release profile have been demonstrated in primary experiments. The release of model antibiotic (tetracycline) at different initial loading was studied (FIG. 2C). NPs were imaged using transmission/scanning electron and atomic force microscopies. Here, we developed a series of particles based on poly(lactic-co-glycolic) acid (PLGA) (due to its controlled biodegradation and compact structure) with a wide range of sizes with the use of microfluidic platform, and determine how size and monodispersity may affect release and effectiveness of antibiotic (tetracycline) drug over time. In addition, the manipulation of molecular weight of PLGA and the ratio between lactic acid and glycolic acid in PLGA copolymer will offer further control of the release profile. Since the regeneration of periodontal tissues will take 2-6 weeks, we target>2 weeks of drug release in this project. This targeted release period can be easily tailored in our drug delivery system as needed. NPs were loaded with three different levels of drugs (5, 15, 20 wt_{drug}/wt_{pLGA}%) as representative low, medium, and high dose of antibiotics. The stability of the NPs in the phosphate buffered saline (PBS) and salivary environment were monitored through dynamic light scattering (DLS) and zeta potential measurements (Zetasizer Nano, Malvern). Natural saliva has a complex and variable composition, and the use of an artificial saliva with known composition should therefore facilitate the understanding of the influence of the salivary constituents on the stability and release of antibiotics from designed NPs. Artificial saliva was prepared, as described before. Briefly, the following salts (mg) were dissolved in 1 L of MilliQ water: 125.6 NaCl, 963.9 KCl, 189.2 KSCN, 654.5 KH₂PO4, 200.0 urea, 336.5 Na₂SO₄, 178.0 NH₄Cl, 227.8 CaCl₂.2H₂O, and 630.8 NaHCO₃. The pH was adjusted to 6.8 by bubbling CO₂ gas through the solution before each experiment. The release kinetics and in vitro characteristics of these NPs studied under gentle shaking at 37° C. in PBS and in artificial saliva. To increase the production rate of PLGA NPs, microvortex platform or parallel flow focusing system were also developed to reach to a productivity of ˜1 g/hour/chip while providing the expected reproducibility and homogeneity of NPs.

NPs can be prepared containing one or more antibiotics, one or more cytokines, or any combination thereof. Preparation of such NPs is described in the examples below.

### Example 2

### Optimization of Patch with Removable Biodegradable Microneedles

The general scheme for casting the patch is shown in FIG. 3A. Alginate, chitosan, and methacrylated gelatin polymers (due to their biocompatibility as well as ease of use and modify) were tested as microneedle core materials (as Polymer 1 in FIG. 3). These polymers have been used before to fabricate microneedle patches for wide range of small molecule or protein transdermal delivery. Alginate generally has slow biodegradation (several months) but amylase present in human saliva can facilitate hydrolysis of the polysaccharide, including alginate, in the mouth environment. Here, we produced, optimized and characterized solid microneedle patches based on these polymers. For example, different concentrations of alginate (0.5-5 wt. %) mixed with the drug-loaded NPs and casted over polydimethylsiloxane (PDMS) molds with designated dimension. These molds were fabricated at clean room facilities of UCLA California NanoSystem Institute (CNSI) or purchased from commercial resources like Blueacre Technology Inc., Ireland. After drying the alginate inside molds, a gelatin layer (FIG. 3; Polymer 2) containing water-soluble tetracycline-HCl (FIG. 3B) formed to cover the molds as a supporting film (air dry) to make the microneedle easy to handle and also to provide burst release of antibiotics inside pocket upon dissolution of this support layer. The dried gelatin film will provide proper mechanical strength as support a material. Gelatin layer dissolves at body temperature in <15 minutes and releases encapsulated tetracycline. Microneedles were dried at room temperature for 20 hours and then removed for the molds. Crosslinking of alginate-based microneedles using various concentrations (e.g., 5-100 mM) of calcium chloride following by second cycle of drying in room temperature was performed after removing the needles from the mold to make microneedles with various stiffness and mechanical properties.

In FIG. 3C, a patch is depicted with cytokine microparticles in polymer 1. Polymer 2 may optionally contain a soluble antibiotic.

The microneedle size and shapes were analyzed using scanning electron microscopy (SEM) (FIG. 4B; one microneedle shown by enlargement) as well as fluorescent microscopy (FIG. 4C) and compared between different formulations. Needles were made from 1.5% alginate and stained with trypan blue (2%) are shown in FIG. 4A. The microneedle dimension (height: 400-1200 μm; base diameter: 150-500 μm) and composition optimized to tune the desired mechanical properties. FIG. 4E depicts the mechanical property of microneedles with and without PLGA nanoparticles. The mechanical (compression) properties of the microneedles were measured by an Instron (Model 5542) based on the procedures explained previously. FIG. 4E shows a slight decrease of mechanical property of microneedles loaded with PLGA NPs due to disturbing the alginate crosslinking networks. The stiffness of the microneedles tuned to the level sufficient for tissue penetration by adjusting the concentration of alginate and Ca²⁺. Degradation of microneedles (FIG. 4F) at physiologically relevant conditions, 37° C., at either (artificial) saliva or PBS was also tested to optimize the formulation. As tissue/mouth pH fluctuate during the normal conditions or during the periodontal disease, effects of pH (pH: 3.0 to 9.5) on release and degradation of microneedles was assessed. The degradation study performed to measure the degradation rate of the material in PBS, artificial saliva and also in pooled human saliva (Innovative Research, Inc. Novi, Mich.) at 37° C. over 8 weeks on a shaker incubator. The results demonstrate the significant difference in degradation rate of alginate-based biomaterials in PBS and human saliva (FIG. 4F).

We also characterized the penetration and adhesion of the patches to the extracted rat's gingival tissues using cryosectioning, staining, and analyzing of the tissue (see Example 3). We also evaluated the patches on extracted pig gums showing successful penetration of placement of microneedles after dissolution of their gelatin-based support layer (FIG. 5).

We also tested the in vitro biocompatibility of the selected microneedle patch candidates with rat gingival mesenchymal stem cells (rGMSCs). rGMSCs were isolated and cultured using the same protocol as described for human-derived GMSCs. Based on these in vitro experiments, we selected optimized formulations that are non-cytotoxic (viability>90%) and support cellular activities (cell proliferation). The material formulations that meet these criteria were used for the in vivo biocompatibility test.

### Example 3

### In Vivo Biocompatibility and Degradation of the Engineered Patches in Rat Periodontal Tissue

The success of this delivery platform depends on its proper degradation profile and biocompatibility. In particular, we examined the degradation rate and host response for implanted microneedle patches in the absence of drug. To test this, we utilized rat periodontal implantation model in presence of microneedle (without drug). For implantation, anesthesia was induced and maintained with isoflurane. Freshly prepared samples were implanted by firmly attaching to periodontal tissues. After 7, 14 and 28 days, the constructs explanted (if not fully degraded) and analyzed for biocompatibility and biodegradation. For the in vivo biocompatibility test, we tested three groups of materials: 1) Sham, and 2) Alginate (two formulations for each selected from the in vitro tests). Each implant type followed up for four different time periods: 7, 14 and 28 days. Number of animals per group to be n=8 based on Power Analysis. We selected formulations, which showed the lowest inflammatory response and degraded between 21 to 28 days for the further studies. Our data shows subcutaneously implanted alginate constructs not caused any immune response as there is no sign of lymphocyte infiltration (CD3) and macrophage (CD68) at day 7.

### Example 4

### Characterization and Optimization of Antibiotics Release

In order to add antibacterial activity to the patches, each patch (including gelatin support layer and alginate needles) was loaded with various dosages to provide three different levels of tetracycline for low (50 μg/patch), medium (300 μg/patch), and high-dose (1.5 mg/patch) delivery. Due to higher drug loading capacity of gelatin base, ≈80% of drug was loaded there to provide strong early stage antibacterial effect. Results confirm that we can load up to 1.5 mg tetracycline per patch which meet the required dose for human use. For example, state-of-the-art Arestin® (Valeant Pharma Inc.) microspheres contain 1 mg of minocycline per therapy. Here, multistage release of antibiotic was achieved by a burst release during the dissolution of gelatin supporting film in less than 15 minutes followed by prolonged release over 4 weeks. Our observations revealed the advantage of using microfluidic NPs over traditionally fabricated (bulk) NPs or free drug encapsulation in alginate-based needles (FIG. 6). The kinetics of drug release from microneedles with different composition and geometry were studied in vitro using HPLC via a modified Franz cell method.

### Example 5

### In Vitro Evaluation of Antibacterial Properties

In our studies, we demonstrated that our full patches have antibacterial properties against periodontal pathogens P.g. (FIG. 7). Here we utilized P.g. and A.a. for the analysis of antibacterial properties. A direct quantitative evaluation of the antimicrobial activity of designed patches were performed. Patches were placed in 2 ml brain heart infusion (BHI) broth inoculated with P.g. or A.a. at an initial concentration optical density at 600 nm (OD600) of 0.3. To control the accuracy of bacterial density, bacteria serially diluted in PBS to 10³, 104, 10⁵, and 10⁶ colony forming units (CFU/ml) and plated overnight (200 μl/plate). Suspensions then incubated at 37° C. for 24, 72, and 120 hours and the change in OD600 was monitored. Assessment of direct inhibition of bacterial growth also performed by counting CFU. LB-agar plates prepared by spreading a 0.5 McFarland (optical density at 600 nm of approximately 0.08-0.1) suspension of bacteria (P.g. or A.a.) in broth using a sterile swab. Bactericidal activity visually assessed to determine the zone of inhibition 1 and 5 days after contact of the patches with the inoculated bacterium. At least four replicates were tested for each experimental group, and then the diameter of the inhibition zone was analyzed.

### Example 6

### Sterilization of Patches

X-ray irradiation using Gulmay Medical RS320 x-ray unit was used to irradiate the fabricated patches before doing any in vitro or in vivo functional assay. We followed the recommended protocol in ISO 11137-2:2013. A sterilization dose of 25 kGy (2.5 Mrads) was used. It has been reported that this dose does not alter the properties of tetracycline. Physical properties, including change in morphology and mechanical stiffness of microneedles patch as well as change in antibacterial were tested after sterilization process. Our results show non-significant change in mechanical properties of alginate patches after receiving one of five cycles of 25 kGy sterilization dose (FIG. 8).

### Example 7

### Design and Optimize Cytokine-Loaded Mesoporous Silica Microparticles with Enhanced Affinity

Recently we have shown that presence of heparin can significantly increase the affinity of positively charged proteins, in one embodiment those with an isoelectric point (pI)>7.5. In this example, heparin-functionalized mesoporous microparticles (10 μm in diameter) are synthesized and optimized to encapsulate target cytokines (IL-4; pI: 9.17) and growth factors (TGF-β; pI: 8.5). These particles are then loaded in a microneedle patch as described above. Monodisperse mesoporous silica microparticles (5 to 50 μm) produced via a microfluidic jet spray-drying route, using cetyl trimethylammonium bromide (CTAB) and/or Pluronic F127 as templating agents, and tetraethylorthosilicate (TEOS) for silica. Heparin-based conjugates (silica-heparin) developed at several conjugation densities (FIG. 9C). Carbodiimide chemistry (NHS/EDC) utilized to modify silica conjugates with heparin after treating the silica with (3-Aminopropyl)triethoxysilane (APTES) to provide primary amine groups (FIG. 9A,B). Heparin presence provides enhanced efficiency and stability of cytokine binding (as exemplified in FIG. 9D) that enables precise spatiotemporal control over the release profile of target proteins (IL-4 and/or TGF-β). IL-4 and TGF-β proteins (5-100 nM) dissolved in 500 μl PBS and then loaded into mesoporous silica microparticles by overnight incubation at 4° C. Particles were then washed with PBS and stored at 4° C. prior use. Absorption (binding) of cytokines was studied after dissolving the microparticles in hydrofluoric acid using ELISA kits (R&D Systems Minneapolis, Minn.) for each protein individually.

### Example 8

### Fabrication of Cytokine Delivery Patches and Study of Cytokines Release

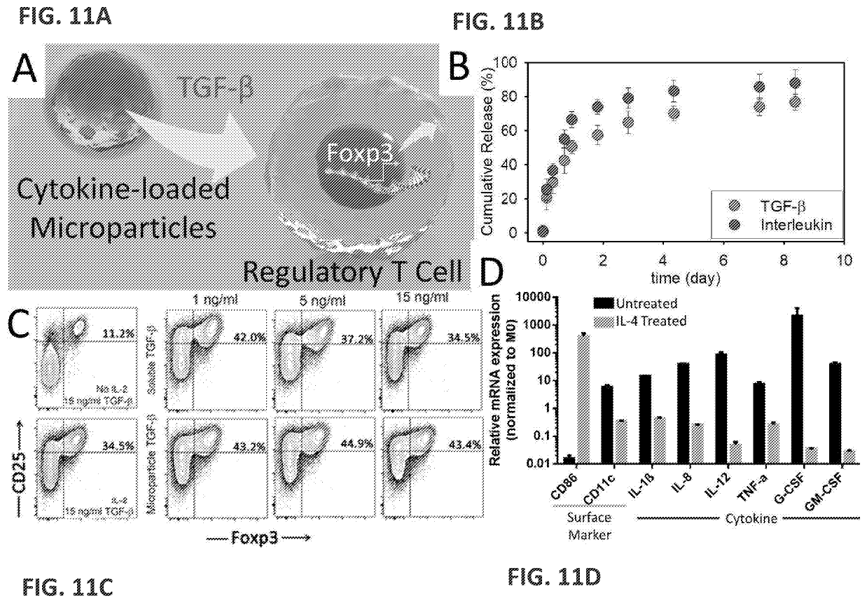

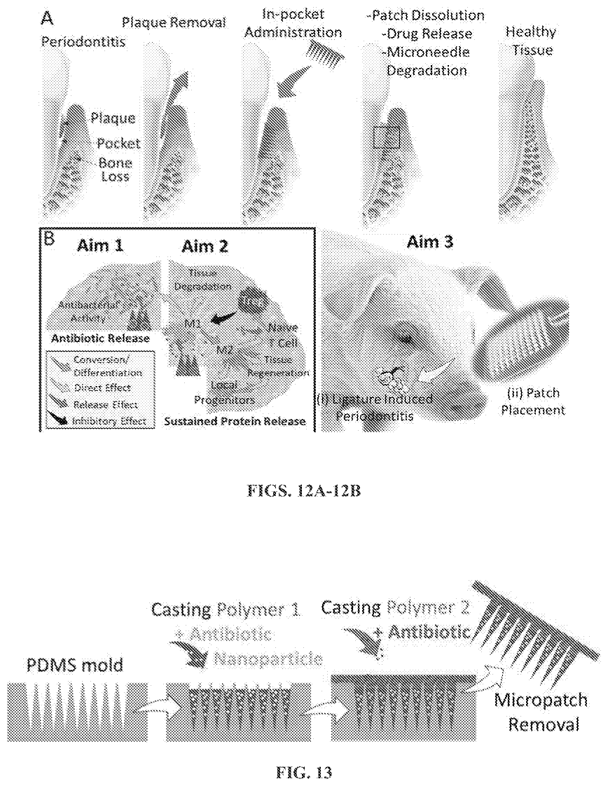

For high quality periodontal treatment, the periodontal patch should act as an immunomodulator by delivering small molecules drugs and/or bioactive molecules to control infection (e.g., with antibiotics), modulate inflammation (e.g., FIG. 11A) and induce the recovery of alveolar bone. Here we used same method as described in Approach 1 to fabricate microneedles (FIG. 10A). IL-4- or TGF-β-loaded silica-heparin microparticles mixed with polymer (e.g., alginate) solution and cast into the molds to generate the microneedle patches. The kinetics of cytokine release from microneedles with different composition and geometry studied using ELISA for each protein individually and in combination to assess the possible protein-protein interaction. We characterized the microstructure of the generated microneedle patch containing cytokines (FIG. 10B) and their mechanical properties at different loading densities as described in Approach 1. Our results confirming the successful encapsulation of cytokine-loaded silica particles into alginate microneedles and the release characteristics. As noted above, the presence of these particles also increased the mechanical properties of resulted microneedles by 7-fold (FIG. 10C). The chemical formulation of microneedles was further modified for co-encapsulation of both small molecule antibiotics (encapsulated in PLGA NPs) as well as cytokines (encapsulated in heparin-silica microparticles). To characterize the release profile of loaded proteins, microparticles alone or encapsulated (in microneedles) with varying level of protein loading incubated in 500 μL of artificial saliva in 48-well plates on a rotational shaker at 37° C. for up to four weeks. At selected time intervals (every 12 hour), the medium was collected and analyzed for released proteins using ELISA Kits. At the end of release study, the remaining proteins extracted from the particles/needles by dissolving them and the percentage of cumulative released proteins measured according to methods explained before. The released percentage measured based on the ratio of the amount released at each time point to the amount that was initially loaded. Based on these studies, we select the formulations that provided controlled release over 2-4 weeks. Our results show the prolonged release of IL-4 and TGF-β from designed platforms (FIG. 11B).

### Example 9

### In Vitro Assessment of Immunomodulatory Properties of Patches

Costar transwell inserts (pore size 1 μm) was used for assessing the effectiveness of the released factor(s) on the polarization of macrophages and differentiation of T cells. Patches containing cytokines (4 experimental groups: no drug, IL-4 alone, TGF-β alone and IL-4/TGF-β) placed in the upper chamber. The bottom chamber contains artificial saliva modified media and 10⁵ of either primary monocytes or naïve T cells or both (5×10⁴). The isolation of primary monocytes was performed as described. Naïve T cells isolated using EasySep Rat T Cell Isolation Kit (STEMCELL Technologies) according to manufacturer's protocol. The change in macrophage phenotypes tested at different time points (day 1-5) using flow cytometry by checking the surface markers (M1): major histocompatibility complex class II (MHC II), CCR7, CD80 and CD86; M2: CD163 (M2c) and CD206 (M2a)). The change in macrophage function characterized by phagocytosis capability, inducible nitric oxide synthase (iNOS) production and inflammatory secretome (M1: IL-1-β, tumor necrosis factor (TNF), IL-6, and IL-12; M2: IL-4, IL-10, and TGF-β) analysis. Quantitative PCR (qPCR) was used to determine the gene expression of these inflammatory cytokines and cellular markers (as exemplified in FIG. 11D). Activation and differentiation of T cells were tested by considering expression of CD25 surface activation marker (FIG. 11C) as well as intracellular expression of the transcription factor Foxp3. In addition, soluble cytokines used as controls to compare the bioactivity of encapsulated proteins. There was >5 independent experiments for all conditions. Our studies show successful reprograming of M1 (and M0) macrophages toward M2 upon treatment with IL-4 (FIG. 11D). We also tested the ability of controlling differentiation of naïve T-cells into induced Treg by providing sustained release TGF-β from alginate-based particles. We cultured rigorously naïve CD4+ T-cells with anti-CD3 and anti-CD28 for 4 days in the wells containing the microparticles loaded with TGF-β at different concentrations and compared them to the soluble (no particles) counterparts at identical concentrations. The expression of Foxp3 was measured by flow cytometry. In all concentrations of TGF-β as provided by the particles, Foxp3 expression was much higher. On the other hand, we found that at very high soluble concentrations of TGF-β, Foxp3 expression plateaued and was even slightly suppressed. Furthermore, the MFI of Foxp3 directly relates to the suppressive capacity of the regulatory T-cells. We found that the MFI of Foxp3 was highest when using particles to deliver TGF-β. These results show that induced regulatory T-cells can be potently generated using microparticles that secrete TGF-β. We varied the dose of IL-4 and TGF-β to reach an optimal level to maximize the conversion into M2 type macrophages. The effects of released IL-4 and TGF-β on macrophages and T cells isolated from male, female, young and old rats were investigated and compared.

### Example 10

### In Vitro Assessment of the Effects on Gingival Cells

To study how alteration of macrophage polarization and regulatory T cell formation may affect host cells in periodontal tissue, we cultured GMSCs in the absence or presence of treated macrophages/T cells in a Transwell co-culture system with macrophages/T cells in the top well for various lengths of time. Before co-culture experiment, these macrophages/T cells were treated with IL-4/TGF-β-containing (or no-drug control) microneedles for a time period that shows effective conversion into M2 macrophages (in 2.3). After 2 days co-culture of macrophages, T cells, and GMSCs, we examined the survival, proliferation, and matrix production (Collagen I) of GMSCs in vitro. Cellular viability and metabolic activity were measured over the course of experiment using Live/Dead Assay Kit and Presto Blue assay, respectively. Collagen I production also assayed by quantitative polymerase chain reaction (qPCR). We found that the optimized delivery platform can enhance the functions of host GMSCs for tissue regeneration.

Thus, successful fabrication of the immunomodulatory patch with tunable characteristics was shown. We observed, by changing the type and concentration of loaded cytokine concentration and heparin conjugation density, the polarization of macrophages and formation of regulatory T cells can be tuned in vitro. We also anticipate that this platform can be expanded to load and deliver wide variety of therapeutic proteins.

For some therapeutic proteins, we found that the activity maybe decreased during the fabrication process. To address such an issue, we load cytokines along with trehalose as a protein chaperon. This compound is being used as a sweetener in products such as chewing gum. Here we used its critical property that helps stabilize proteins as a biological preservative. Also, use of support polymers like polyvinylpyrrolidone instead of trehalose tested. In order to facilitate the recovery of lost bone tissue, incorporation of recombinant bone morphogenetic protein-2 (rBMP-2) also investigated.

### Example 11

### Optimization of Periodontal Patches with Detachable and Biodegradable Microneedles for Multistage Antibiotic Delivery

Engineering an effective microneedle patch for the treatment of periodontitis requires a combination of physical characteristics including tunable drug loading/release and degradation profile. In addition, adhesion and retention of the drug delivery platform at the application site is important for predictable outcomes. For successful periodontal treatment and tissue regeneration, the patch should act as an immunomodulator by delivering small molecules and/or bioactive factors. We also observed that simple antibiotic loading and release was not effective, as there is limited transgingival drug penetration. In this project, we engineered a novel periodontal delivery platform inside the tissue. We then incorporated small molecule drugs and cytokines into the delivery platform. Therefore, we demonstrated that multistage release of antibiotics through (i) burst release at the pocket during dissolution of the membrane support and (ii) sustained release inside the periodontal tissue from the degradable microneedles containing antibiotic-loaded nanoparticles (NPs) enabled us to overcome bacterial infection with minimal drug dosage for improved disease management. Tetracycline is the model drug, herein, as it is commonly used clinically to combat human periodontal pathogens. Delivery of other antibiotics like doxycycline and minocycline also evaluated.

### Experimental Design

### To Design and Optimize Antibiotic-Loaded Monodisperse NPs to Prolong Delivery.

Here we first created small and monodisperse NPs with a microfluidic technique. This well-controlled mixing regimen can be precisely adjusted on microfluidic platforms (primarily through flow ratio and velocity), allowing monodisperse NP generation with tunable structural features, improved drug encapsulation efficiency, and adjustable release pattern. In this study, we fabricated monodisperse NPs with a microfluidic technique as well with traditional bulk mixing. The possibility of controlling size (60-200 nm), zeta potential (1-14 mV), drug loading efficiency (>95%), and sustained release profile has been proven in our prior experiments, as described above. Release of the model antibiotic (tetracycline) at different initial loading of 5-20 wt % was studied for up to three weeks. Here, we developed a series of particles based on poly(lactic-co-glycolic) acid (PLGA) (due to its controlled biodegradation and compact structure) with a wide range of sizes and use of a microfluidic platform, and determine how size and monodispersity may affect antibiotic (tetracycline) release and effectiveness over time. In addition, PLGA molecular weight manipulation and the ratio between lactic acid and glycolic acid in a PLGA copolymer offered further control of the release profile. Since initial periodontal tissue regeneration will take at least 2-6 weeks, we targeted>one week drug release in this platform. This targeted release period can be further tailored in our drug delivery system as needed. NPs were loaded with three different drugs levels (5, 15, 20 wt_{drug}/wt_{PLGA}%) as representative low, medium, and high antibiotic dosages. NP stability in PBS and saliva was monitored through dynamic light scattering and zeta potential measurements (Zetasizer Nano, Malvern). Natural saliva has a complex and variable composition, so using artificial saliva with a known composition should facilitate understanding salivary constituent influence on antibiotic stability and release from designed NPs. Artificial saliva was prepared, as previously described. Briefly, the following salts (mg) were dissolved in 1 L of MilliQ water: 125.6 NaCl, 963.9 KCl, 189.2 KSCN, 654.5 KH₂PO₄, 200.0 urea, 336.5 Na₂SO₄, 178.0 NH₄Cl, 227.8 CaCl₂.2H₂O, and 630.8 NaHCO₃. pH adjusted to be 6.8 by bubbling CO₂ gas through the solution before each experiment. NP release kinetics and in vitro characteristics was studied under gentle shaking at 37° C. in PBS and in artificial saliva.

To Optimize a Periodontal Patch with Removable Biodegradable Microneedles.

Alginate biopolymer (due to its biocompatibility as well as ease of use and modification) as well as gelatin methacryloyl (GelMA) were tested as microneedle core materials (as Polymer 1 in FIG. 13). These polymers has been used previously to fabricate microneedle patches for wide range of small molecule or protein transdermal delivery.

Alginate generally has slow biodegradation (several months) but amylase present in human saliva can facilitate polysaccharide hydrolysis, including alginate, in the oral environment. Here, we produced, optimize and characterize solid microneedle patches using polymers. Different alginate (0.5-5 wt. %) or GelMA (5-25 wt. %) concentrations were mixed with drug-loaded NPs and casted over polydimethylsiloxane molds with designated dimensions. In the case of using GelMA, the patches were incubated under 50 W/cm2 ultraviolet light for 5 min. After 1 day of drying in dark at room temperature, the gelatin layer (FIG. 13; Polymer 2) containing water-soluble tetracycline-HCl were formed to cover the molds as a supporting film (air dry) to make the microneedle easy to handle and also to provide a burst release of antibiotics inside the periodontal pocket upon support layer dissolution.

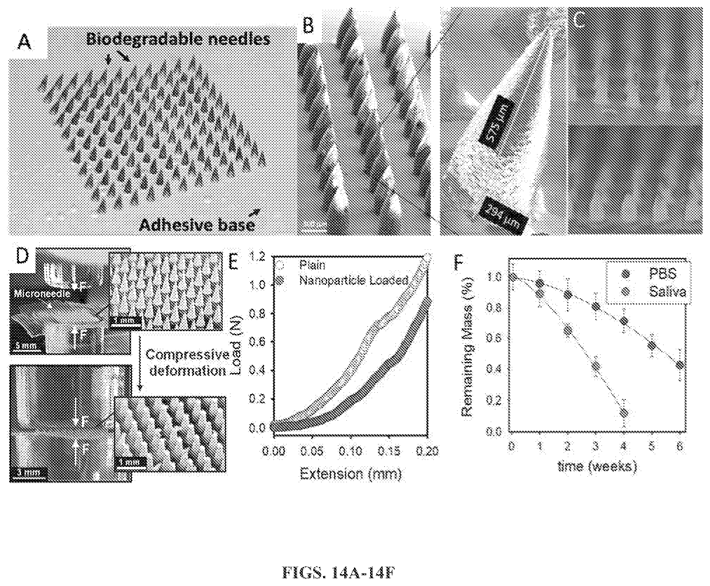

The dried gelatin film provides proper mechanical strength as a support material. The gelatin layer will be dissolved at body temperature in <15 minutes and release encapsulated tetracycline. Microneedles were dried at room temperature for 20 hours and then removed for the molds (FIG. 14A).

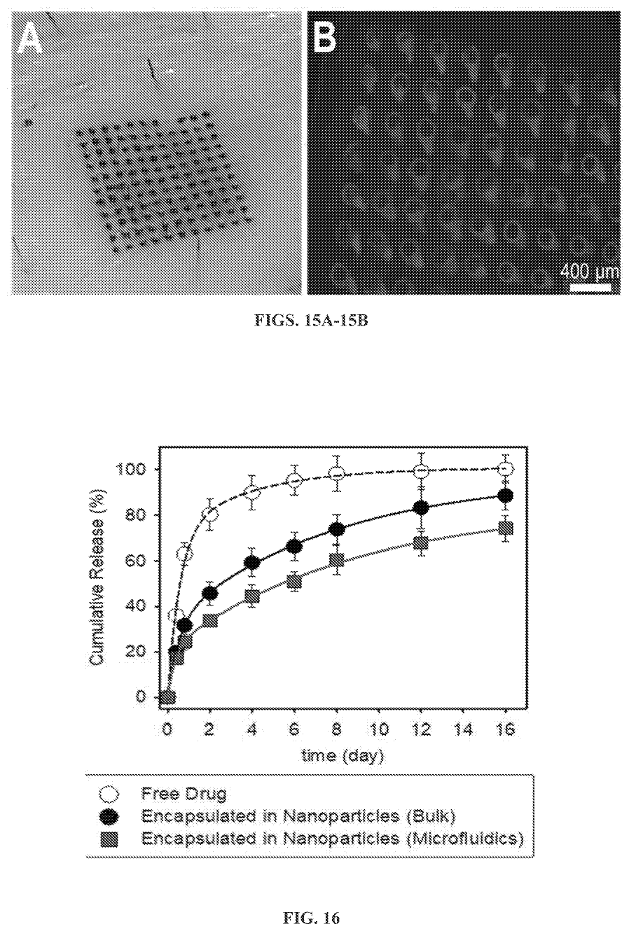

In the case of alginate-based microneedles, we crosslinked microneedles using various calcium chloride concentrations (e.g. 10-50 mM), followed by a second drying cycle at room temperature which was performed after removing needles from the mold to make microneedles with varied stiffness and mechanical properties. Microneedle size and shape were analyzed by scanning electron (FIG. 14B) and fluorescent microscopy (FIG. 14C) and compared between different formulations. Microneedle dimension (height: 400-850 μm; base diameter: 150-400 μm) and composition was optimized to tune the mechanical properties (FIG. 14E) and degradation profile. FIG. 14E shows a slight decrease in microneedle mechanical properties loaded with PLGA NPs due to disturbing alginate crosslinking networks. Microneedle stiffness was tuned to the level sufficient for tissue penetration by adjusting alginate and Ca²⁺ concentration. Microneedle degradation at physiologically relevant conditions (37° C.) in either (artificial) saliva or PBS was tested to optimize the formulation. As tissue/oral pH will fluctuate greatly in healthy conditions or in periodontitis, pH (pH: 3.0 to 9.5) effects on microneedle release and degradation was assessed. Degradation studies also be performed to measure the degradation rate of the material in PBS, artificial saliva, and in pooled human saliva (Innovative Research, Inc. Novi, Mich.) at 37° C. for 8 weeks on a shaker incubator. Our preliminary results demonstrate significant differences in the degradation rate of alginate-based biomaterials in PBS vs. human saliva (FIG. 14F), which was further investigated. Microneedles mechanical (compression) properties was measured by an Instron (Model 5542) based on previously published procedures. We also characterized patch penetration and adhesion to extracted pig gingival tissues after sectioning, staining, and analyzing the tissue. Our preliminary results from extracted minipig gingiva showed successful tissue penetration after microneedle placement following gelatin-based support layer dissolution (FIG. 15). In vitro biocompatibility also tested utilizing selected microneedle patch candidates with pig gingival mesenchymal stem cells (pGMSCs), as previously described in our rat studies. pGMSCs were isolated and cultured using the same protocol as described for human-derived GMSCs. Based on these in vitro experiments, we selected candidate formulations that are non-cytotoxic (viability>90%) and support cellular activities (cell proliferation). Material formulations that meet these criteria were used for in vivo biocompatibility studies.

### Characterization and Optimization of Antibiotic Release.

To add antibacterial activity to the patches, each patch (including gelatin support layer and alginate needles) was loaded with various dosages to provide three different levels of tetracycline for low (100 μg/patch), medium (300 μg/patch), and high-dose (1 mg/patch) delivery. Due to higher drug loading capacity of the gelatin base, ≈80% of drug was loaded to provide strong early antibacterial effects. Our preliminary results confirm that up to 1.5 mg tetracycline can be loaded per patch, which will meet the required dose for human use. Commercially available Arestin® (Valeant Pharma, Canada) microspheres contain 1 mg minocycline per therapy. Arestin® can deliver the drug in the periodontal pocket, but without adherence to subgingival tissues. Here, antibiotic multistage release was achieved by a burst release during the gelatin supporting film dissolution in <15 minutes followed by prolonged release over 4 weeks into gingiva and the surrounding periodontium. Our preliminary observations revealed the advantage of using microfluidic NPs over traditionally fabricated (bulk) NPs or free drug encapsulation in alginate-based needles (FIG. 16). Drug release kinetics from microneedles with different composition and geometry were studied in vitro using HPLC via a modified Franz cell method. Target delivery regimens were reached for each dose by modifying formulation and composition.

### In Vitro Evaluation of Antibacterial Properties.

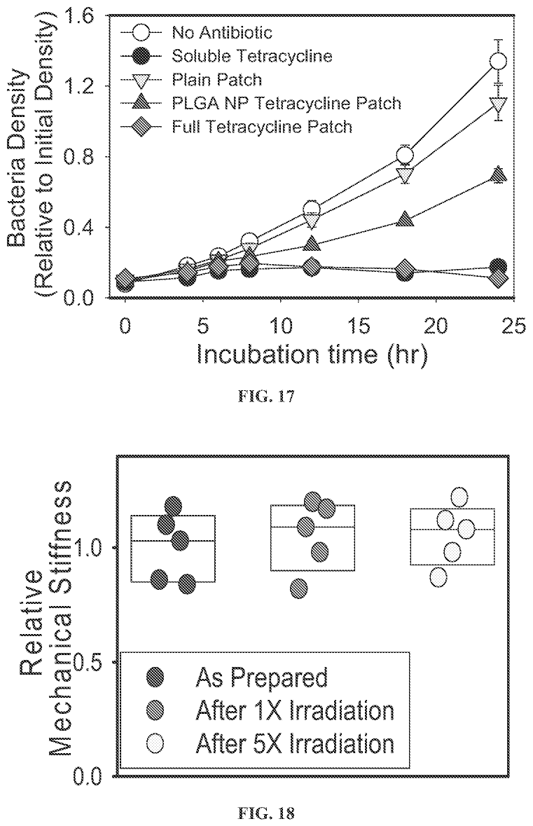

In our studies, we demonstrate that our full patches have antibacterial properties against periodontal pathogens P.g. (FIG. 17). Here we utilized P.g. and A.a. for antibacterial property analysis. A direct quantitative evaluation of the antimicrobial activity of designed patches was performed. Patches were placed in 2 ml brain heart infusion broth inoculated with P.g. or A.a. at an initial concentration OD600 of 0.3. To control the accuracy of bacterial density, bacteria serially diluted in PBS to 10³, 10⁴, 10⁵, and 10⁶ colony forming units (CFU/ml) and plated overnight (200 μl/plate). Suspensions were incubated at 37° C. for 24, 72, and 120 hours and change in OD600 were monitored. Assessment of direct bacterial growth inhibition also performed by counting CFUs. LB-agar plates were prepared by spreading a 0.5 McFarland (optical density at 600 nm of approximately 0.08-0.1) bacteria suspension (P.g. or A.a.) in broth using a sterile swab. Bacteria inhibition was visually assessed to determine the zone of inhibition 1 and 5 days after patch contact with the inoculated bacterium. At least four replicates were performed for each experimental group, and the inhibition zone diameter was analyzed.

### Sterilization of Patches.

X-ray irradiation (Gulmay Medical RS320 x-ray unit) was used to irradiate the fabricated patches before in vitro or in vivo functional assays, following ISO 11137-2:2013 recommended protocols (30). A 25 kGy (2.5 Mrads) sterilization dose was used, since this dose does not alter tetracycline properties. Physical properties, including changes in morphology and mechanical stiffness of the microneedle patch, or antibacterial property change after sterilization, were tested. Our results show non-significant changes in alginate patch mechanical properties after receiving three cycles of 25 kGy (FIG. 18).

### Analysis of Outcome.

We successfully fabricated removable microneedle patches that can control tetracycline release. By changing the polymer composition, drug concentration, and microfabricated mold architecture, we were able to optimize the delivery platform to effectively penetrate gingival tissues and release its cargo at target sites. We demonstrated that the initial tetracycline burst release, followed by its sustained release over 2-4 weeks, could reduce bacterial content. Moreover, the polymer type and its properties (e.g., molecular weight, crosslinking density) were optimized to provide a prolonged release profile, which is advantageous for the proposed application. We observed that the compressive strength of the alginate needles will increase by enhancing calcium and polymer concentrations. We did not see any significant change in properties of the patches or antibiotic after sterilization. High cell viability (>85%) is achieved for the engineered microneedles. Here we developed alginate and GelMA to make microneedles; however, degradation and mechanical properties of other polymers such as hyaluronic acid and chitosan can also be tested as alternative microneedle materials (32-35). The microneedle patches can be mass produced at a cost that should be similar to or less than the cost of a needle and syringe.

### Example 12

### To Optimize an Engineered Microneedle Patch for the Delivery of Immunomodulatory Cytokines and Assess its Function In Vitro

### Rationale

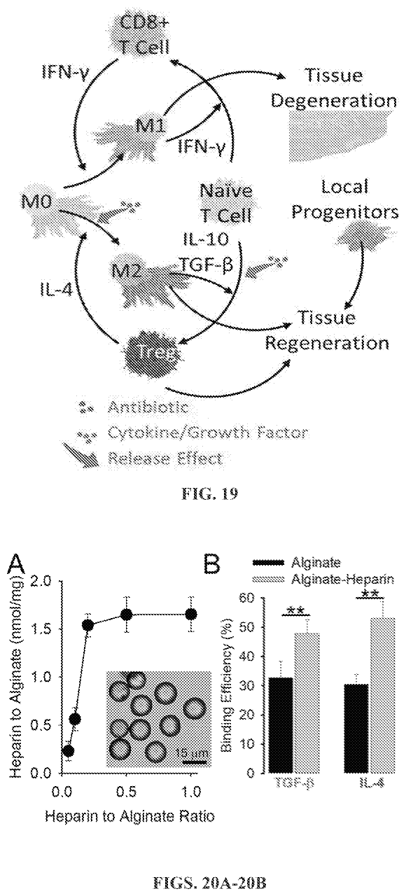

Although microbial infection is the initial factor of periodontitis, accumulation of host immune cells into the periodontium plays a critical role in gingival and alveolar bone degeneration and the progression of disease. Host immune cells including macrophages and T cells play a significant role in the host's defense responding to periodontal bacterial infection. Activated macrophages not only phagocytose periodontal pathogens, but also secrete cytokines that result in alveolar bone resorption, and damage periodontal connective tissue via secretion of matrix metalloproteinases (MMPs) and collagenase. Many reports suggest that monocytes entering an inflammatory environment first polarize into classically activated macrophages, M1 macrophages, which are involved in pro-inflammatory activation, mediating host defense against bacteria, and then switch to M2, alternatively activated macrophages, which displays anti-inflammatory and pro-healing functions. Polarized M1 and M2 macrophages can, to some extent, switch from one phenotype to another upon microenvironmental (e.g., chemokine/cytokine composition) changes. In periodontitis, a phenotypic switch occurs from M2 to M1 in the periodontium and serum, which causes alveolar bone destruction. Modulation of the immune environment via several soluble factors (cytokines) can alter the polarization of activated macrophages. It has been well established that IL-4 (and IL-10) can induce the conversion of macrophages from M1 to M2 type and the delivery of these cytokines promote nerve and muscle regeneration. IL-4 was determined by meta-analysis to be the only cytokine decreased in chronic periodontitis patients and elevated after periodontal treatment, indicating that IL-4 has protective effects in periodontitis. In addition, IL-4 can downregulate pro-inflammatory cytokines and restrain osteoclastogenesis. Conversely, the adaptive immune system will react to infection and inflammation by activating T cells. Activated T cells can secret inflammatory cytokines (e.g. IFN-γ), which push macrophage polarization to form more M1 type, causing greater inflammation. However, presentation of certain cytokines (e.g., IL-4 and IL-10) and transforming growth factor beta (TGF-β) can control inflammation by differentiating naïve T cell into induced regulatory T cells (Treg) that can repolarize macrophages toward anti-inflammatory (M2) lineages. Thus, we demonstrated that by incorporation of immunomodulatory cytokines (e.g., IL-4) and/or growth factors (e.g., TGF-β) into the engineered microneedle-based patches and providing sustained and prolonged delivery in periodontal tissues, we can alter the immunogenic microenvironment by macrophage repolarization toward the anti-inflammatory/pro-healing (M2) lineage, formation of regulatory T cells, and regeneration of periodontal tissue (periodontium) (FIG. 19), following the antibiotic delivery to kill bacteria.

### Experimental Design

To Design and Optimize Cytokine-Loaded Alginate-Heparin Microparticles with Enhanced Affinity.

Recently we have shown that sustained cytokines release from polymeric microparticles can regulate cellular fate, and the presence of heparin significantly increases affinity of positively charged proteins, isoelectric point (pI)>7.5. Here, heparin-functionalized alginate as well as heparin-functionalized mesoporous silica microparticles (15 μm in diameter) were synthesized and optimized to encapsulate target cytokines (IL-4; pI: 9.17) and growth factors (TGF-β; pI: 8.5). These particles were loaded in microneedle patches. Monodisperse alginate-heparin and silica-heparin microparticles (10-50 μm) were produced via a microfluidic droplet generation and microjet spray platforms, respectively. Heparin-based conjugates were developed at several conjugation densities (FIG. 20A). Carbodiimide chemistry (NHS/EDC) was utilized to modify amine-modified alginate or APTES modified mesoporous silica. Heparin presence will provide enhanced efficiency and cytokine binding stability (as exemplified in FIG. 20B) to enable precise spatiotemporal control over the target protein release profiles (IL-4 and/or TGF-β). IL-4 and TGF-β protein (10 nM, 25 nM, 50 nM; concentrations of each protein) were dissolved in 500 μl PBS and loaded into heparinized microparticles by 4° C. overnight incubation. Particles were washed with PBS and stored (4° C.). Cytokine absorption (binding) were studied after dissolving microparticles in EDTA or hydrofluoric acid using ELISA (R&D Systems, Minneapolis, Minn.) for each protein.

### Fabrication of Cytokine Delivery Patches and Study of Cytokines Release.

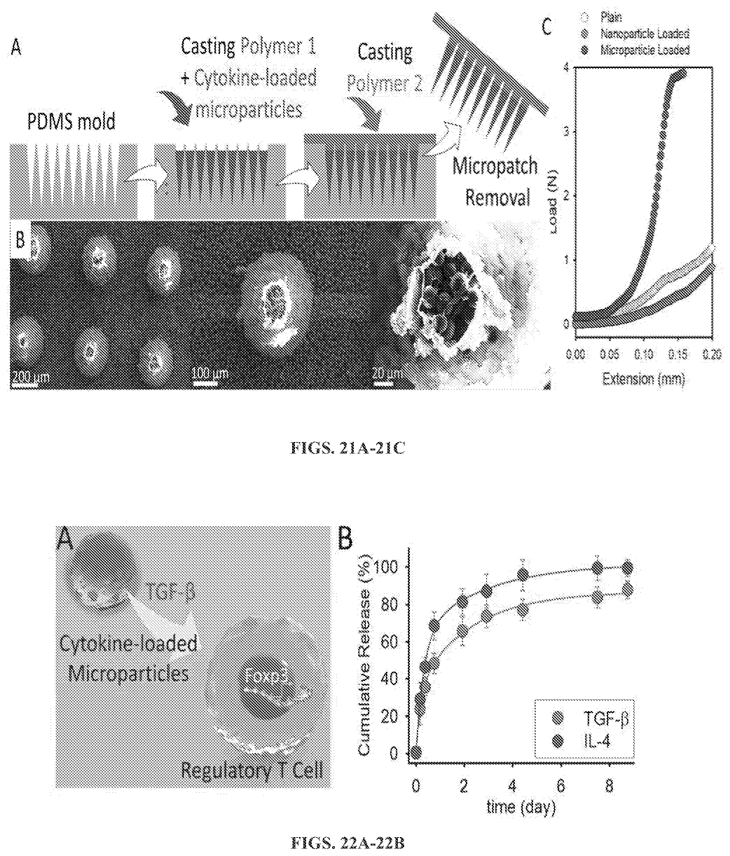

For effective periodontal treatment, the periodontal patch should act as an immunomodulator by delivering small molecules, drugs and/or bioactive molecules to control infection, modulate inflammation, and induce alveolar bone regeneration. Here we used the same method as described in Aim 1 to fabricate microneedles. IL-4- and/or TGF-β-loaded alginate-heparin microparticles were mixed with alginate/GelMA solution and casted into the molds (as described in Aim 1; FIG. 21A) to generate microneedle patches. Cytokine release kinetics from microneedles with different composition and geometry was studied using ELISA for each protein individually and combined to assess possible protein-protein interactions. We characterized the microstructure of the generated microneedle patches containing cytokines and their mechanical properties at different loading densities as described in Aim 1. Our results confirmed successful encapsulation of cytokine-loaded alginate particles into alginate microneedles (FIG. 21B). Presence of these particles will also increase the mechanical properties of resulted microneedles by 7-fold (FIG. 21C).

The microneedle chemical formulation can be further modified for co-encapsulation of both small molecule antibiotics (encapsulated in PLGA NPs) and cytokines (encapsulated in alginate-heparin microparticles), which was integrated and addressed in Aim 3. Cytokine loading contents can be modulated by encapsulating different concentrations (IL-4 or TGF-β) inside microneedle patches (FIG. 22A). Our results show that 50-1000 pg of either cytokine can be loaded per needle, which will provide 5-100 ng cytokine per patch. It has been reported that 40 ng local IL-4 administration per treatment reduces periodontal disease progression and helps regenerate bone in a rat model. To characterize loaded protein release profiles, microparticles alone or encapsulated (in microneedles) with varying protein levels were incubated in 500 μL of artificial saliva in 48-well plates on a rotational shaker at 37° C. for up to four weeks. At selected time intervals (every 12 hours), media was collected and analyzed for released proteins using ELISA. At the end of the release experiment, remaining proteins were extracted from the particles/needles by dissolving them and the percentage of cumulative released proteins were measured according to published methods. The released percentage was measured based on the ratio of protein released at each time point to the initially loaded protein. Based on these studies, we selected formulations providing controlled release over 2-4 weeks. Our results show prolonged release of IL-4 and TGF-β from designed platforms (FIG. 22B).

### In Vitro Assessment of Immunomodulatory Properties of Patches.

Costar transwell inserts (pore size 1 μm) were used to assess the effectiveness of released factor(s) on macrophage polarization and T cell differentiation. Patches containing cytokines (Experimental groups: no drug, IL-4 alone, TGF-β alone, and IL-4/TGF-β) were placed in the upper chamber. The lower chamber contains artificial saliva modified media and 10⁵ of primary monocytes, naïve T cells, or both (5×10⁴). Primary monocyte isolation was performed as described. Naïve T cells was isolated using EasySep T Cell Isolation Kit (StemCell Technologies) according to manufacturer's protocol Change in macrophage phenotype was tested at different time points (day 1-5) using flow cytometry by checking surface markers; (M1: major histocompatibility complex class II (MHC II), CCR7, CD80 and CD86; M2: CD163 (M2c) and CD206 (M2a)) Change in macrophage function was characterized by phagocytosis capability, inducible nitric oxide synthase (iNOS) production, and inflammatory secretome (M1: tumor necrosis factor (TNF), IL-6, and IL-12; M2: IL-4, IL-10, and TGF-β) analysis. Quantitative PCR (qPCR) was used to determine gene expression of inflammatory cytokines and cellular markers (as exemplified in FIG. 23A-23B). In addition, soluble cytokines were used as controls to compare the bioactivity of encapsulated proteins. Our studies show successful reprograming of M1 (and M0) macrophages toward M2 upon treatment with IL-4 (FIG. 23A-23B).

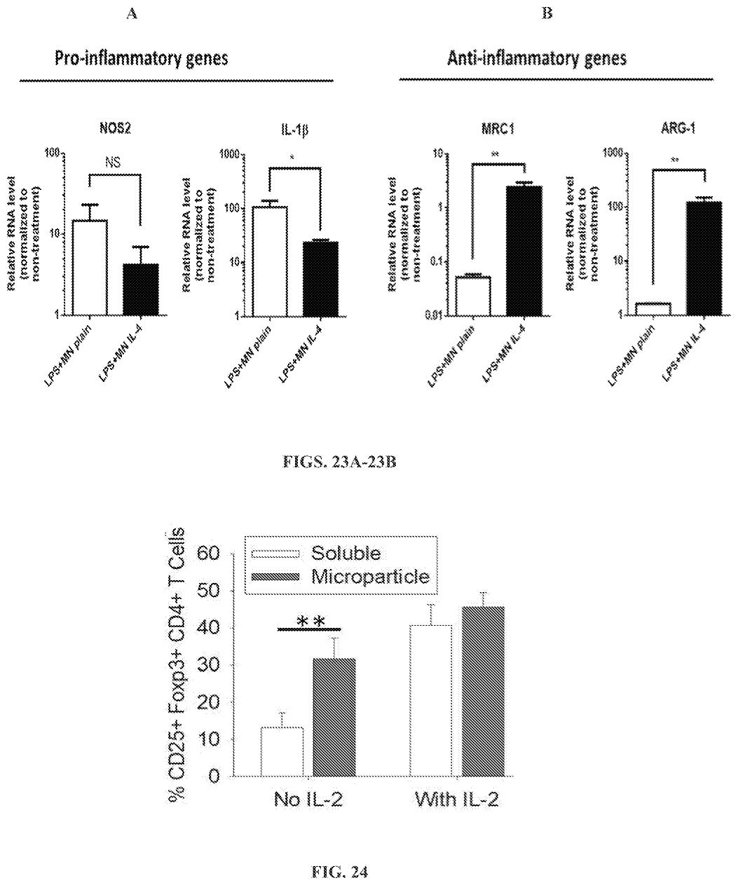

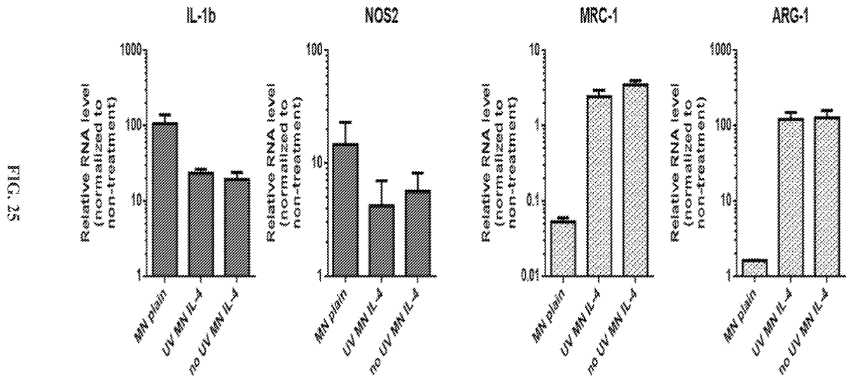

T cell activation and differentiation was tested by expression of CD25 surface activation marker and intracellular expression of the transcription factor Foxp3. We tested the ability of controlling naïve T-cell differentiation into induced Tregs by providing sustained TGF-β release from microneedles (as exemplified in FIG. 24). Here, we cultured rigorously naïve CD4+ T-cells with anti-CD3 and anti-CD28 for 4 days in wells containing microneedles loaded with TGF-β at different concentrations and compared them to soluble (no microneedles) counterparts at identical concentrations. Foxp3 expression was measured by flow cytometry (FIG. 24). In all concentrations of TGF-β as provided by the particles, Foxp3 expression was significantly increased. We found that the Foxp3 expression was highest when using microneedles to deliver TGF-β (FIG. 24). Results show that induced regulatory T-cells can be potently generated using microneedles that secrete TGF-β. We also varied the IL-4 and TGF-β dose to reach an optimal level to maximize conversion into M2 macrophages as well as Treg formation. The effects of released IL-4 and TGF-β on macrophages and T cells isolated from mice/rats/pigs/humans were investigated, and the findings was used to correlate with the therapeutic effects in vivo in Example 13.

We also tested how UV exposure during the crosslinking of GelMA-based microneedles may affect the stability of the loaded cytokines. As shown in FIG. 25, UV irradiation did not cause significant change in protein (here IL-4) stability and its function when in being released from microneedles patch.

### Analysis of Outcome

Successful fabrication of the immunomodulatory patch with tunable characteristics was shown. We demonstrated that, by changing the type and concentration of loaded cytokine concentrations and heparin conjugation density, the macrophage polarization and regulatory T cell formation can be tuned in vitro. We expect that this platform can be expanded to load and deliver a wide variety of therapeutic proteins, and that we can optimize the delivery platform to enhance the host cellular function for tissue regeneration.

### Example 13

### To Evaluate the Functionality of Engineered Microneedle Patches In Vivo

### Rationale

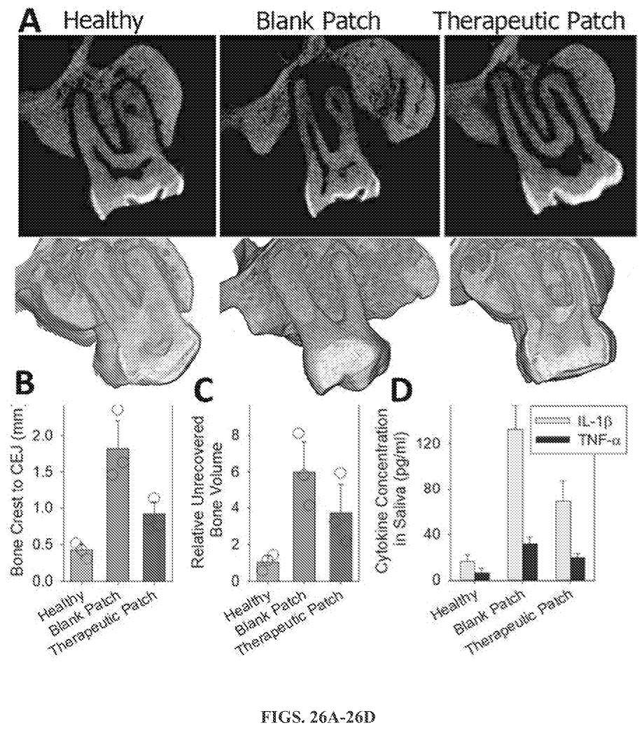

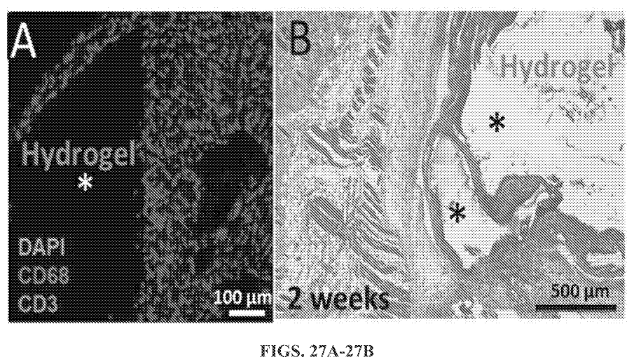

To assess the effectiveness of the engineered drug-delivery platform in the treatment of periodontal disease, we also investigated the engineered microneedle patch for periodontal tissue regeneration. We have demonstrated bone regeneration in rats with ligature induced periodontitis utilizing our immunomodulatory and antimicrobial microneedle patches (FIG. 26). Here, we shown the optimized the microneedle patches in the more clinically relevant minipig model. We demonstrated that delivery of a sustained release antibiotic and immunomodulatory cytokines will decrease inflammatory markers, increase the presence of M2 macrophages, and improve alveolar bone regeneration. Minipigs are representative of humans with respect to anatomy, structure, healing, and remodeling after adult-ness, and therefore are suitable to investigate periodontal disease and regeneration. The pig gingival sulcus is 2-3 mm, and the width of attached gingiva are also similar to humans Periodontal disease occurs in pigs, consisting of clinically swollen gingiva, accumulated plaque and calculus, bleeding on probing, and increased pocket depths, as well as histological features such as inflammatory cells in gingival tissues, and alveolar bone destruction. In addition, minipigs avoid the ethical concerns of dogs and other large animal models, making them ideal for therapeutic and regenerative strategies proposed herein.

### Experimental Design

### In Vivo Biocompatibility and Degradation of the Engineered Patches in Pig Submucosal Tissues.

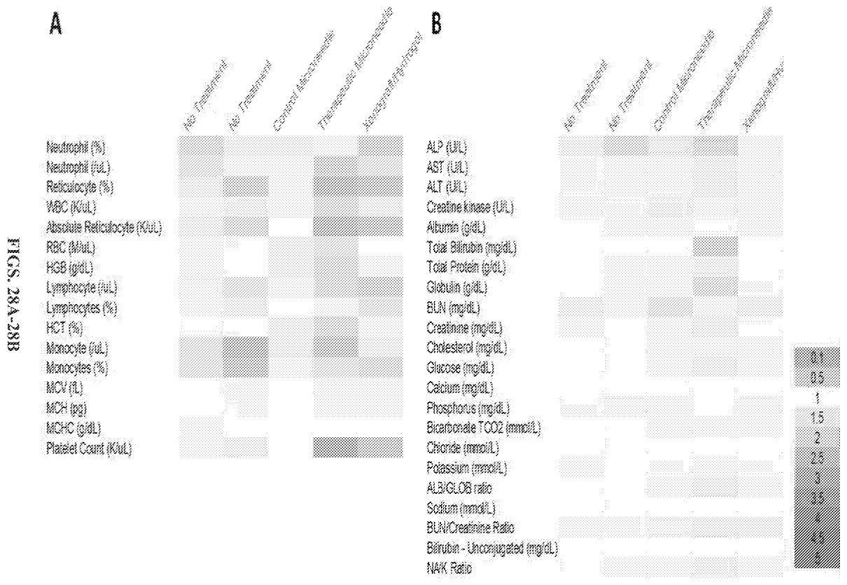

Success of this delivery platform depends on its proper degradation profile and biocompatibility. In particular, we examined the degradation rate and host response for implanted microneedle patches in the absence of the drug. To test this, microneedles implanted into the submucosa of pigs. After the induction of general anesthesia, freshly prepared samples were implanted submucosally by firmly attaching to underlying tissues, in the buccal mucosa of the maxilla and mandible bilaterally, approximately 1 cm apical to the teeth. Two weeks later, the constructs explanted and analyzed for biocompatibility and biodegradation. For the in vivo biocompatibility testing, we also evaluated three groups of materials: 1) sham, and 2, 3) drug-free microneedle patch (two formulations selected based on in vitro studies). The implant type examined at the two-week time point, which should demonstrate local safety and biocompatibility. Animal numbers per group (n=2 pigs; 8 implantations per animal; 2 sites per quadrant of the right and left maxilla and mandible, anterior and posterior) are based on a power analysis. Histological analysis was performed by staining for inflammatory cell markers (e.g., CD45, CD68 for macrophages and CD3 for lymphocytes) and apoptotic cells (using TUNEL assay kit from Abcam Inc.). Our data in rats demonstrates no immunological response after subcutaneous implantation of alginate constructs, by the lack of lymphocyte (CD3) and macrophage (CD68) infiltration at day 7 (FIG. 27A-27B).

### Treatment of Ligature-Induced Periodontal Disease in Minipigs

To induce periodontal disease in minipigs, anesthetized and intubated Yucatan minipigs (Jackson Labs, Bar Harbor, Me.) undergone 3-0 silk ligature placement and bacteria delivery around the maxillary 3^{rd} and 4^{th} premolars, and 1^{st} molars to induce periodontal disease (PD), while the contralateral side utilized as a split mouth control or other treatment arm (FIG. 29A). The ligature model, along with human periodontal pathogens (P.g. or A.a.) induces periodontitis by 4-8 weeks, without resolution of the defect upon ligature removal. After 4-week induction of PD, in vivo CT scans (Siemens Somatom Definition AS CT Scanner, Baton Rouge, La.) was performed on a 64-slice scanner in the prone position. The scan parameters were standardized (120 kVp, 500 mA with automatic mA optimization at a noise index of 15, median mA 490, collimated slice thickness 0.625 mm, total detector width 55 mm, rotation speed 0.4 s, and table feed per rotation 55 mm, resulting in a scan speed of ˜3 s for 30 cm scan length in the z-axis. Volumetric data was converted to DICOM format and imported in Amira Imaging software to generate 3D and multiplanar reconstructed images to make linear measurements. The distance from the alveolar crest to the CEJ was measured at the sagittal plane of the 3^{rd} and 4^{th} premolars and 1^{st} molars to evaluate periodontal disease bone levels for comparison to subsequent therapeutic interventions. After imaging, a mucoperiosteal flap was made and the periodontal pocket was debrided of inflammatory tissue. Microneedle patches loaded with optimal antibiotic and cytokine concentrations placed onto the tissues and sutured with 4-0 vicryl. These patches can deliver up to 1 mg tetracycline and up to 80 ng of either cytokine. Our studies in pigs demonstrate clinically relevant periodontal disease including gingival inflammation and alveolar bone loss. We also were able to manipulate microneedle patches in the surgical site, demonstrating no technical problems with the procedures (FIG. 29A-29B). Baseline probing depths, bleeding on probing (mild, moderate, severe), plaque index (PLI), sulcus bleeding index (SBI), gingival recession (GR), and attachment loss (AL) were measured using a Williams periodontal probe (Hu-Friedy, Chicago, Ill.). At 2, 4, and 8 weeks after microneedle insertion, animals anesthetized for objective clinical measurements as above, as well as gingival crevicular fluid collection to evaluate inflammation.

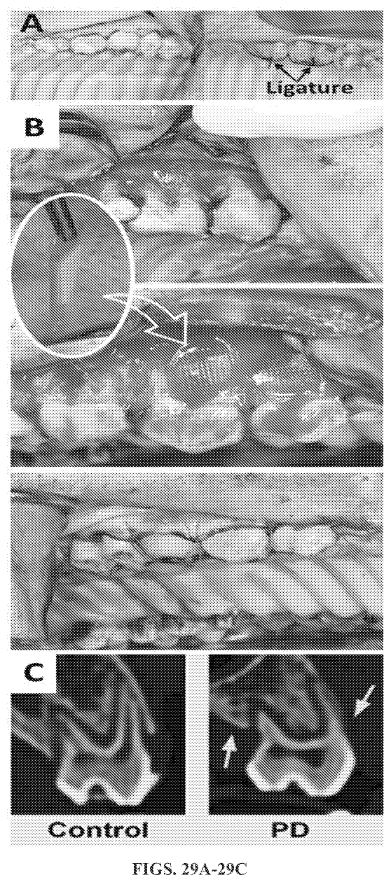

To confirm safety in this large animal model, we performed whole blood analysis to evaluate complete blood count, comprehensive metabolic screening, and liver function tests at 1, 3, 7, 14, and 30 days after patch placement. With our optimized PD, tetracycline- and cytokine-loaded microneedle patch, we also included 2 female pigs to test sex as a biological variable. Results demonstrate that use of microneedle patches will not cause systematic toxicity as tested by analyzing the blood from minipigs treated with therapeutic or control microneedles (FIGS. 28A-28B).

### The Effects of Drug Delivery on Inflammatory Responses

Pro-inflammatory cytokines (IL-1β, TNF-α, and IL-8) are known as inflammatory markers of periodontal tissue condition. Here we compared the levels of these molecules in gingival crevicular fluid (GCF) from rats and pigs treated with different microneedle formulations using a split-mouth design, as performed in human clinical studies. At different time points (2, 4 and 8 weeks) post-implantation, animals were anesthetized (not intubated) and GCF was collected for 30 seconds with periopaper strip (Proflow, Amityville, NY). Following elution of the fluid, IL-1β, TNF-α, and IL-8 ELISA assays were carried out. Chemokine concentrations were calculated and compared with samples from healthy and untreated diseased sites. Our results in rats demonstrate significant (p<0.05) reduction in IL-1β and TNF-α secretion four weeks after combinatorial (microneedle patches containing both tetracycline- and cytokine-loaded particles) insertion compared to sham (FIG. 26D). In addition, to evaluate the type and population of macrophages and T cells, treated and untreated periodontal disease sites were stained for M1, M2 and T cells markers to assess local inflammatory response. Markers for M1 macrophages (CCR7, CD80, and CD86), M2 macrophages (CD163 and CD206), and T cells (CD3, CD4, CD8, CD25, Foxp3) also evaluated on decalcified tissues by immunohistochemistry.

### Effects of Drug Delivery on Bone Regeneration

At the 12-week time point, in vivo CT scans were performed as described above (FIG. 29C). After in-vivo CT imaging, animals were euthanized, and jaws were dissected, and fixed in 10% formalin for 48 hours. Whole maxillae was subjected to cone-beam CT (CBCT) scanning (3D Accuitomo 170 Scanner, J Morita, Irvine, Calif.) with consistent exposure factors (90 kVp and 6 mA with a 17.5 sec exposure time, during 360 rotation and field of view 10×14 cm with 0.25 mm isometric voxel). Scans were imported into in vivo software (Anatomage) for multiplanar axial, sagittal, coronal and 3D reconstructions.

Volumetric analyses of bone volume, tissue volume, BV/TV, trabecular number, thickness, and separation were performed, as well as linear measurements of periodontal bone loss/regeneration, alveolar bone height. These CBCTs significantly higher resolution (100 μm) for radiographic evaluation and provide clinical relevance for future patient-oriented trials. Following CT analyses, maxillae was demineralized, paraffin embedded, and 5-μm thick sections were prepared at a corrected sagittal plane through the interproximal molar spaces. Sections on the right and left maxilla was stained by hematoxylin and eosin (H&E), and heights of the newly regenerated periodontal ligament-like tissue and bone were measured at three different positions from the buccal to the lingual side. To determine drug delivery effects on matrix remodeling in gingival tissues, H&E staining and Masson's Trichrome staining for collagen and elastin were used to examine matrix production.

### Analysis of Outcome

We demonstrated that our engineered patches can successfully reduce the bacterial content and promote gingival tissue and alveolar bone regeneration. We also showed tissue integration and degradation of microneedles in mucosal tissue would occur without causing cytotoxicity. We demonstrated minimal inflammatory reactions around defect sites, or systemically. We revealed that by application of cytokine-loaded microneedles, we can regenerate periodontal defects to confirm the important role of macrophages, biomaterials, localized delivery in periodontal tissues, and inductive signals for achieving tissue regeneration. Our results in rats show significant (p<0.05) bone recovery four weeks after combinatorial microneedle patch insertion compared to sham (FIG. 26B, 26C). We also observed significant periodontal tissue and alveolar bone regeneration at 12 weeks in minipig experiments.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A periodontal drug delivery system comprising a microneedle patch, the microneedle patch comprising:
a. a detachable support layer comprising a surface capable of a sealable application to a diseased periodontal tissue of a subject in need thereof, wherein the detachable support layer comprises a first drug, and the detachable support layer is formulated for immediate release of the first drug; and
b. a plurality of detachable, transdermal microneedles having bases disposed on the surface of the detachable support layer and projecting perpendicularly therefrom, wherein the transdermal microneedles comprise nano/microparticles loaded with a second drug, and the transdermal microneedles are formulated for sustained release of the second drug.

2. The periodontal drug delivery system of claim 1, wherein the detachable support layer, the transdermal microneedles, or both, independently comprise a biodegradable polymer.

3.-4. (canceled)

5. The periodontal drug delivery system of claim 1, wherein the detachable support layer is non-membranous and non-adhesive.

6. The periodontal drug delivery system of claim 1, wherein the detachable support layer, the microneedles, or both are porous.

7. The periodontal drug delivery system of claim 1, wherein the first drug is an antibiotic or an antibacterial agent.

8. The periodontal drug delivery system of claim 7 wherein the first drug is present within the detachable support layer, the first drug is coated on the detachable support layer, or a combination thereof.

9. The periodontal drug delivery system of claim 1, wherein the first drug is dispersed within a biodegradable polymer.

10. The periodontal drug delivery system of claim 1, wherein the nano/microparticles loaded with the second drug are dispersed within a biodegradable polymer.

11. The periodontal drug delivery system of claim 1, wherein the second drug is the same drug as the first drug, is a different drug than the first drug, or combinations thereof.

12. The periodontal drug delivery system of claim 11, wherein the different drug is a small molecule, an antibiotic, an immunomodulatory cytokine, a growth factor or combinations thereof.

13. (canceled)

14. The periodontal drug delivery system of claim 12, wherein the small molecule, antibiotic, immunomodulatory cytokine growth factor or combinations thereof is encapsulated in the nano/microparticles, wherein the encapsulated nano/microparticles are dispersed within a biopolymer.

15. The periodontal drug delivery system of claim 14, wherein the biopolymer comprises alginate and calcium chloride.

16.-23. (canceled)

24. The periodontal drug delivery system of claim 1, wherein the drug-loaded transdermal microneedles have a height ranging from about 300 μm to about 1200 μm.

25. The periodontal drug delivery system of claim 1, wherein the drug-loaded transdermal microneedles comprises a base diameter of from about 100 μm to about 400 μm.

26. The periodontal drug delivery system of claim 1, wherein the drug-loaded transdermal microneedles have an aspect ratio of height to diameter of about 5 or more.

27.-36. (canceled)

37. The periodontal drug delivery system of claim 1, wherein the second drug loaded in the transdermal microneedles comprises microparticles comprising an anti-inflammatory agent, a cytokine, a growth factor, or a combination thereof, and the detachable support layer comprises an antibiotic.

38. The periodontal drug delivery system of claim 1, wherein the second drug loaded in the transdermal microneedles comprises an anti-inflammatory agent, and the detachable support comprises an antibiotic.

39. The periodontal drug delivery system of claim 1, wherein the second drug loaded in the transdermal microneedles comprise a cytokine, and the detachable support comprises an antibiotic.

40.-44. (canceled)

45. The periodontal drug delivery system of claim 1, wherein the diseased periodontal tissue comprises periodontal ligament, cementum, gingiva, and/or alveolar bone.

46. The periodontal drug delivery system of claim 1, wherein the subject has periodontitis and/or gingivitis.

47. A method for regenerating periodontal tissue in a subject in need thereof, the method comprising sealably applying to the periodontal tissue the periodontal delivery system of claim 1.

48.-184. (canceled)

185. A method for treating periodontitis and/or gingivitis in a subject in need thereof, the method comprising sealably applying to the periodontal tissue a microneedle patch, the microneedle patch comprising the periodontal delivery system of claim 231.

186.-230. (canceled)

231. A periodontal drug delivery system comprising a microneedle patch, the microneedle patch comprising:
a. a detachable support layer comprising a surface capable of a sealable application to a diseased periodontal tissue of a subject in need thereof; and
b. a plurality of detachable, drug-loaded transdermal microneedles having bases disposed on the surface of the detachable support layer and projecting perpendicularly therefrom.

232. The periodontal drug delivery system of claim 231, wherein the detachable support layer, the transdermal microneedles, or both, independently comprise a biodegradable polymer.

233.-234. (canceled)

235. The periodontal drug delivery system of claim 231, wherein the detachable support layer is non-membranous and non-adhesive.

236. The periodontal drug delivery system of claim 231, wherein the detachable support layer, the microneedles, or both are porous.

237. The periodontal drug delivery system of claim 231, wherein the detachable support layer comprises a drug.

238. The periodontal drug delivery system of claim 237 wherein the drug is present within the detachable support layer, the drug is coated on the detachable support layer, of the combination thereof.

239. The periodontal drug delivery system of claim 231, wherein the transdermal microneedles are coated with a drug.

240. The periodontal drug delivery system of claim 231, wherein the drug-loaded transdermal microneedles have a height ranging from about 300 μm to about 1200 μm.

241. The periodontal drug delivery system of claim 231, wherein the drug-loaded transdermal microneedles comprises a base diameter of from about 100 μm to about 400 μm.

242. The periodontal drug delivery system of claim 231, wherein the drug-loaded transdermal microneedles have an aspect ratio of height to diameter of about 5 or more.

243.-244. (canceled)

245. The periodontal drug delivery system of claim 231, wherein the drug-loaded transdermal microneedles comprise an antibiotic, antibacterial agent, an anti-inflammatory agent, a growth factor, a cytokine, or any combination thereof.

246. (canceled)

247. The periodontal drug delivery system of claim 237, wherein the drug in the detachable support layer comprises an antibiotic, antibacterial agent, an anti-inflammatory agent, a growth factor, a cytokine or any combination thereof.

248.-251. (canceled)

252. The periodontal drug delivery system of claim 237, wherein the drug-loaded transdermal microneedles comprise an antibiotic, an anti-inflammatory agent, a cytokine, a growth factor, or a combination thereof, and the detachable support layer comprises an antibiotic.

253. (canceled)

254. The periodontal drug delivery system of claim 237, wherein the drug-loaded transdermal microneedles comprise an anti-inflammatory agent, and the detachable support comprises an antibiotic.

255. The periodontal drug delivery system of claim 237, wherein the drug-loaded transdermal microneedles comprise a cytokine, and the detachable support comprises an antibiotic.

256. The periodontal drug delivery system of claim 245, wherein the antibacterial agent is tetracycline, doxycycline, chlorhexidine and/or minocycline.

257. The periodontal drug delivery system of claim 245 comprising an amount of from about 50 ng up to about 1,500 ng of the antibacterial agent per transdermal microneedle patch.

258. The periodontal drug delivery system of claim 245, wherein the anti-inflammatory agent is a steroid and/or a non-steroidal anti-inflammatory drug.

259.-260. (canceled)

261. The periodontal drug delivery system of claim 231, wherein the diseased periodontal tissue comprises periodontal ligament, cementum, gingiva, and/or alveolar bone.

262. The periodontal drug delivery system of claim 231, wherein the subject has periodontitis and/or gingivitis.

263. A method for regenerating periodontal tissue in a subject in need thereof, the method comprising sealably applying to the periodontal tissue a microneedle patch, the microneedle patch comprising the periodontal drug delivery system of claim 231.

264.-294. (canceled)

295. A method for reducing local inflammation in periodontal tissue of a subject in need thereof, the method comprising sealably applying to the periodontal tissue a microneedle patch, the microneedle patch comprising the periodontal drug delivery system of claim 231.

296.-326. (canceled)

327. A method for promoting regeneration of bone loss in periodontal tissue of a subject in need thereof, the method comprising sealably applying to the periodontal tissue a microneedle patch, the microneedle patch comprising the periodontal drug delivery system of claim 1.

328.-358. (canceled)

359. A method for treating periodontitis and/or gingivitis in a subject in need thereof, the method comprising sealably applying to the periodontal tissue a microneedle patch, the microneedle patch comprising the periodontal drug delivery system of claim 1.

360. The method of claim 359, wherein the detachable support layer, the transdermal microneedles, or both, independently comprise a biodegradable polymer.

361.-389. (canceled)

390. A method for promoting regeneration of bone loss in periodontal tissue of a subject in need thereof, the method comprising sealably applying to the periodontal tissue a microneedle patch, the microneedle patch comprising the periodontal drug delivery system of claim 231.

391. A method for reducing local inflammation in periodontal tissue of a subject in need thereof, the method comprising sealably applying to the periodontal tissue a microneedle patch, the microneedle patch comprising the periodontal drug delivery system of claim 1.

392. The periodontal drug delivery system of claim 247, wherein the antibacterial agent is tetracycline, doxycycline, chlorhexidine and/or minocycline.

393. The periodontal drug delivery system of claim 247, wherein the anti-inflammatory agent is a steroid and/or a non-steroidal anti-inflammatory drug.
